(19) United States
(12) Patent Application Publication
(10) Pub. No.: US 2007/0185089 A1
(43) Pub. Date: Aug. 9, 2007

(54) Benzazepine derivatives for the treatment of neurological and psychiatric disorders

(75) Inventors: Mark James Bamford, Essex (GB); Thomas Daniel Heightman, Essex (GB); David Matthew Wilson, Essex (GB); Jason Witherington, Essex (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(73) Assignee: Glaxo Group Limited, Greenford Middlesex (GB)

(21) Appl. No.: 10/598,759

(22) PCT Filed: Mar. 10, 2005
(86) PCT No.: PCT/GB05/00939

§ 371 Date: Sep. 11, 2006

(30) Foreign Application Priority Data
Mar. 12, 2004 (GB) 0405628.9

(51) Int. Cl. A61K 31/55; C07D 223/16

(52) U.S. Cl. 514/217.02; 540/595

(57) ABSTRACT

The present invention relates to novel benzazepine derivatives of formula (I); wherein: R¹ represents —C₃₋₇ cycloalkyl optionally substituted by C₁₋₃ alkyl; R² represents -aryl, -heterocyclyl, heteroaryl, -aryl-X—C₃₋₈ cycloalkyl, cycloalkyl, -aryl-X-aryl, -aryl-X-heteroaryl, -aryl-X-heterocyclyl, -heteroaryl-X—C₃₋₈ cycloalkyl, -heteroaryl-X-aryl, -heteroaryl-X-heteroaryl, -heteroaryl-X-heterocyclyl, -heterocyclyl-X—C₃₋₈ cyclooalkyl, -heterocyclyl-X-aryl, -heterocyclyl-X-heteroaryl or -heterocyclyl-X-heterocyclyl; X represents a bond, O, CO, —CH₂O—, —COCH₂—, —COCH₂O—, —CONR^{2b}—, —COCH₂NR^{2b}CO—, —CSNH—, SO₂, —SO₂C₁₋₃ alkyl-, —SO₂C₂₋₃ alkenyl-, —COC₂₋₃ alkenyl-, —CO—C(R^{2a})(R^{2b})— or —CO—C(R^{2a})(R^{2b})CH₂—; having pharmacological activity, processes for their preparation, to compositions containing them and to their use in the treatment of neurological and psychiatric disorders.

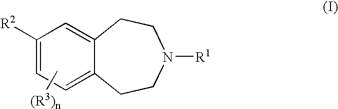

The present invention relates to novel benzazepine derivatives having pharmacological activity, processes for their preparation, to compositions containing them and to their use in the treatment of neurological and psychiatric disorders.

JP 2001226269 and WO 00/23437 (Takeda Chem Ind Ltd) describe a series of benzazepine derivatives which are claimed to be useful in the treatment of obesity. DE 2207430, U.S. Pat. No. 4,210,749 and FR 2171879 (Pennwalt Corp) and GB 1268243 (Wallace and Tiernan Inc) all describe a series of benzazepine derivatives which are claimed as being antagonists for narcotics (such as morphine or codeine) and also anti-histamines and anticholinergic agents. WO 02/14513 (Takeda Chem Ind Ltd) describe a series of benzazepine derivatives with GPR12 activity which are claimed to be useful in the treatment of attention deficit disorder, narcolepsy or anxiety. WO 02/02530 (Takeda Chem Ind Ltd) describe a series of benzazepine derivatives as GPR14 antagonists which are claimed to be useful in the treatment of hypertension, atherosclerosis and cardiac infarction. WO 01/03680 (Isis Innovation Ltd) describe a series of benzazepine derivatives which are claimed as effective agents in the preparation of cells for transplantation in addition to the inhibition of diseases such as diabetes. WO 00/21951 (SmithKline Beecham pic) discloses a series of tetrahydrobenzazepine derivatives as modulators of dopamine D3 receptors which are claimed to be useful as antipsychotic agents. WO 01/87834 (Takeda Chem Ind Ltd) describe a series of benzazepine derivatives as MCH antagonists which are claimed to be useful in the treatment of obesity. WO 02/15934 (Takeda Chem Ind Ltd) describe a series of benzazepine derivatives as urotensin II receptor antagonists which are claimed to be useful in the treatment of neurodegenerative disorders. WO 04/018432 (Eli Lilly and Company) describe a series of substituted azepines as histamine H3 receptor antagonists.

The histamine H3 receptor is predominantly expressed in the mammalian central nervous system (CNS), with minimal expression in peripheral tissues except on some sympathetic nerves (Leurs et al., (1998), Trends Pharmacol. Sci. 19, 177-183). Activation of H3 receptors by selective agonists or histamine results in the inhibition of neurotransmitter release from a variety of different nerve populations, including histaminergic and cholinergic neurons (Schlicker et al., (1994), Fundam. Clin. Pharmacol. 8, 128-137). Additionally, in vitro and in vivo studies have shown that H3 antagonists can facilitate neurotransmitter release in brain areas such as the cerebral cortex and hippocampus, relevant to cognition (Onodera et al., (1998), In: The Histamine H3 receptor, ed Leurs and Timmerman, pp 255-267, Elsevier Science B.V.). Moreover, a number of reports in the literature have demonstrated the cognitive enhancing properties of H3 antagonists (e.g. thioperamide, clobenpropit, ciproxifan and GT-2331) in rodent models including the five choice task, object recognition, elevated plus maze, acquisition of novel task and passive avoidance (Giovanni et al., (1999), Behav. Brain Res. 104, 147-155). These data suggest that novel H3 antagonists and/or inverse agonists such as the current series could be useful for the treatment of cognitive impairments in neurological diseases such as Alzheimer's disease and related neurodegenerative disorders.

The present invention provides, in a first aspect, a compound of formula (I) or a pharmaceutically acceptable salt thereof: 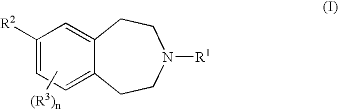
wherein:
R¹ represents —C₃₋₇ cycloalkyl optionally substituted by C₁₋₃ alkyl;
R² represents -aryl, -heterocyclyl, -heteroaryl, -aryl-X—C₃₋₈ cycloalkyl, -aryl-X-aryl, -aryl-X-heteroaryl, -aryl-X-heterocyclyl, -heteroaryl-X—C₃₋₈ cycloalkyl, -heteroaryl-X-aryl, -heteroaryl-X-heteroaryl, -heteroaryl-X-heterocyclyl, -heterocyclyl-X—C₃₋₈ cycloalkyl, -heterocyclyl-X-aryl, -heterocyclyl-X-heteroaryl or -heterocyclyl-X-heterocyclyl;
X represents a bond, O, CO, —CH₂O—, —COCH₂—, —COCH₂O—, —CONR^{2b}, —COCH₂NR^{2b} CO—, —CSNH—, SO₂, —SO₂C₁₋₃ alkyl-, —SO₂C₂₋₃ alkenyl-, —COC₂₋₃ alkenyl-, —CO—C(R^{2a})(R^{2b})— or —CO—C(R^{2a})(R^{2b})CH₂—;
R^{2a} represents hydrogen or C₁₋₆ alkyl;
R^{2b} represents hydrogen, C₁₋₆ alkyl, aryl, heteroaryl, heterocyclyl or C₁₋₆ alkylamido;
R³ represents halogen, C₁₋₆ alkyl, C₁₋₆ alkoxy, cyano, amino or trifluoromethyl;
n is 0, 1 or 2;
wherein said alkyl, cycloalkyl, aryl, heteroaryl and heterocyclyl groups of R² may be optionally substituted by one or more substituents (eg. 1, 2 or 3) which may be the same or different, and which are selected from the group consisting of halogen, hydroxy, cyano, nitro, ═O, haloC₁₋₆ alkyl, haloC₁₋₆ alkoxy, C₁₋₆ alkyl, hydroxyC₁₋₆ alkyl, C₁₋₆ alkoxy, arylC₁₋₆ alkoxy, C₁₋₆ alkylthio, C₁₋₆ alkoxyC₁₋₆ alkyl, C₃₋₇ cycloalkylC₁₋₆ alkoxy, C₁₋₆ alkanoyl, C₁₋₆ alkoxycarbonyl, C₁₋₆ alkylsulfonyl, C₁₋₆ alkylsulfinyl, C₁₋₆ alkylsulfonyloxy, C₁₋₆ alkylsulfonylC₁₋₆ alkyl, sulfonyl, arylsulfonyl, arylsulfonyloxy, arylsulfonylC₁₋₆ alkyl, aryloxy, C₁₋₆ alkylsulfonamido, C₁₋₆ alkylamino, C₁₋₆ alkylamido, —R⁵, —CO₂R⁵, —COR⁵, —C₁₋₆ alkyl-COR⁵, C₁₋₆ alkylsulfonamidoC₁₋₆ alkyl, C₁₋₆ alkylamidoC₁₋₆ alkyl, arylsulfonamido, arylcarboxamido, arylsulfonamidoC₁₋₆ alkyl, arylcarboxamidoC₁₋₆ alkyl, aroyl, arylC₁₋₆ alkyl, aroylC₁₋₆ alkyl, arylC₁₋₆ alkanoyl, or a group —NR⁶R⁷, —C₁₋₆ alkyl-NR⁶R⁷, —C₃₋₈ cycloalkyl-NR⁶R⁷, —CONR⁶R⁷, —NR⁶COR⁷, —NR⁶SO₂R⁷, —OCONR⁶R⁷, —NR⁶CO₂R⁷, —NR⁵CONR⁶R⁷ or —SO₂NR⁶R⁷ (wherein R⁵, R⁶ and R⁷ independently represent hydrogen, C₁₋₆ alkyl, haloC₁₋₆ alkyl, —C₃₋₈ cycloalkyl, —C₁₋₆ alkyl-C₃₋₈ cycloalkyl, aryl, —C₁₋₆ alkyl-aryl, heterocyclyl or heteroaryl, or wherein —NR⁶R⁷ may represent a nitrogen containing heterocyclyl group, and wherein said R⁵, R⁶ and R⁷ groups may be optionally substituted by one or more substituents (eg. 1, 2 or 3) which may be the same or different, and which are selected from the group consisting of halogen, hydroxy, C₁₋₆ alkyl, C₁₋₆ alkoxy, cyano, amino, ═O or trifluoromethyl);
or solvates thereof.

In one aspect of the invention, the substituents present on the alkyl, cycloalkyl, aryl, heteroaryl and heterocyclyl groups of R² are selected from the group consisting of halogen, hydroxy, cyano, nitro, ═O, haloC₁₋₆ alkyl, haloC₁₋₆ alkoxy, C₁₋₆ alkyl, C₁₋₆ alkoxy, arylC₁₋₆ alkoxy, C₁₋₆ alkylthio, C₁₋₆ alkoxyC₁₋₆ alkyl, C₃₋₇ cycloalkylC₁₋₆ alkoxy, C₁₋₆ alkanoyl, C₁₋₆ alkoxycarbonyl, C₁₋₆ alkylsulfonyl, C₁₋₆ alkylsulfinyl, C₁₋₆ alkylsulfonyloxy, C₁₋₆ alkylsulfonylC₁₋₆ alkyl, sulfonyl, arylsulfonyl, arylsulfonyloxy, arylsulfonylC₁₋₆ alkyl, aryloxy, C₁₋₆ alkylsulfonamido, C₁₋₆ alkylamino, C₁₋₆ alkylamido, —R⁵, —CO₂R⁵, —COR⁵, —C₁₋₆ alkyl-COR⁵, C₁₋₆ alkylsulfonamidoC₁₋₆ alkyl, C₁₋₆ alkylamidoC₁₋₆ alkyl, arylsulfonamido, arylcarboxamido, arylsulfonamidoC₁₋₆ alkyl, arylcarboxamidoC₁₋₆ alkyl, aroyl, arylC₁₋₆ alkyl, aroylC₁₋₆ alkyl, arylC₁₋₆ alkanoyl, or a group —NR⁶R⁷, —C₁₋₆ alkyl-NR⁶R⁷, —C₃₋₈ cycloalkyl-NR⁶R⁷, —CONR⁶R⁷, —NR⁶COR⁷, —NR⁶SO₂R⁷, —OCONR⁶R⁷, —NR⁶CO₂R⁷, —NR⁵CONR⁶R⁷ or —SO₂NR⁶R⁷ (wherein R⁵, R⁶ and R⁷ independently represent hydrogen, C₁₋₆ alkyl, haloC₁₋₆ alkyl, —C₃₋₈ cycloalkyl, —C₁₋₆ alkyl-C₃₋₈ cycloalkyl, aryl, heterocyclyl or heteroaryl, or wherein —NR⁶R⁷ may represent a nitrogen containing heterocyclyl group, and wherein said R⁵, R⁶ and R⁷ groups may be optionally substituted by one or more substituents (eg. 1, 2 or 3) which may be the same or different, and which are selected from the group consisting of halogen, hydroxy, C₁₋₆ alkyl, C₁₋₆ alkoxy, cyano, amino, ═O or trifluoromethyl).

In a further aspect of the invention, the substituents present on the alkyl, cycloalkyl, aryl, heteroaryl and heterocyclyl groups of R² are selected from the group consisting of halogen, hydroxy, cyano, nitro, ═O, haloC₁₋₆ alkoxy, C₁₋₆ alkoxy, hydroxyC₁₋₆ alkyl, unsubstituted arylC₁₋₆ alkoxy, C₁₋₆ alkylthio, C₁₋₆ alkoxyC₁₋₆ alkyl, C₃₋₇ cycloalkylC₁₋₆ alkoxy, C₁₋₆ alkylsulfonyl, C₁₋₆ alkylsulfinyl, C₁₋₆ alkylsulfonyloxy, C₁₋₆ alkylsulfonylC₁₋₆ alkyl, sulfonyl, unsubstituted arylsulfonyl, unsubstituted arylsulfonyloxy, unsubstituted arylsulfonylC₁₋₆ alkyl, unsubstituted aryloxy, C₁₋₆ alkylsulfonamido, C₁₋₆ alkylamino, C₁₋₆ alkylamido, —R⁵, —CO₂R⁵, —COR⁵, —C₁₋₆ alkyl-COR⁵, C₁₋₆ alkylsulfonamidoC₁₋₆ alkyl, C₁₋₆ alkylamidoC₁₋₆ alkyl, unsubstituted arylsulfonamido, unsubstituted arylcarboxamido, unsubstituted arylsulfonamidoC₁₋₆ alkyl, unsubstituted arylcarboxamidoC₁₋₆ alkyl, unsubstituted arylcarbonylC₁₋₆ alkyl, or a group —NR⁶R⁷, —C₁₋₆ alkyl-NR⁶R⁷, —C₃₋₈ cycloalkyl-NR⁶R⁷, —CONR⁶R⁷, —NR⁶COR⁷, —NR⁶SO₂R⁷, —OCONR⁶R⁷, —NR⁶CO₂R⁷, —NR⁵CONR⁶R⁷ or —SO₂NR⁶R⁷ (wherein R⁵, R⁶ and R⁷ independently represent hydrogen, C₁₋₆ alkyl, haloC₁₋₆ alkyl, —C₃₋₈ cycloalkyl, —C₁₋₆ alkyl-C₃₋₈ cycloalkyl, aryl, —C₁₋₆ alkyl-aryl, heterocyclyl or heteroaryl, or wherein —NR⁶R⁷ may represent a nitrogen containing heterocyclyl group, and wherein said R⁵, R⁶ and R⁷ groups may be optionally substituted by one or more substituents (eg. 1, 2 or 3) which may be the same or different, and which are selected from the group consisting of halogen, hydroxy, C₁₋₆ alkyl, C₁₋₆ alkoxy, cyano, amino, ═O or trifluoromethyl).

In the context of the present invention, a —C₁₋₆ alkylamidoC₁₋₆ alkyl group includes a —C₁₋₆ alkyl-CO—NH—C₁₋₆ alkyl group and a —C₁₋₆ alkyl-NH—CO—C₁₋₆ alkyl group.

In a further aspect of the invention, X represents a bond, O, CO, —CH₂O—, —COCH₂—, —COCH₂O—, —CONR^{2b}—, —COCH₂NR^{2b} CO—, SO₂, —SO₂C₁₋₁₃ alkyl-, —SO₂C₂₋₃ alkenyl-, —COC₂₋₃ alkenyl-, —CO—C(R^{2a})(R^{2b}) or —CO—C(R^{2a})(R^{2b})CH₂—.

Alkyl groups, whether alone or as part of another group, may be straight chain or branched and the groups alkoxy and alkanoyl shall be interpreted similarly. Alkyl moieties are more preferably C₁₋₄ alkyl, eg. methyl or ethyl. The term ‘halogen’ is used herein to describe, unless otherwise stated, a group selected from fluorine, chlorine, bromine or iodine.

References to ‘aryl’ include references to monocyclic carbocyclic aromatic rings (eg. phenyl) and bicyclic carbocyclic aromatic rings (e.g. naphthyl) or carbocyclic benzofused rings (eg. C₃₋₈ cycloalkyl fused to a phenyl ring, such as dihydroindenyl or tetrahydronaphthalenyl).

The term “heterocyclyl” is intended to mean a 4-7 membered monocyclic saturated or partially unsaturated aliphatic ring or a 4-7 membered saturated or partially unsaturated aliphatic ring fused to a benzene ring, which aliphatic ring contains 1 to 3 heteroatoms selected from oxygen, nitrogen or sulphur. Suitable examples of such monocyclic rings include pyrrolidinyl, piperidinyl, piperazinyl, morpholinyl, thiomorpholinyl, tetrahydrofuranyl, tetrahydropyranyl, diazepanyl, azepanyl, imidazolidinyl, isothiazolidinyl, oxazolidinyl, pyrrolidinone and tetrahydro-oxazepinyl. Suitable examples of benzofused heterocyclic rings include indolinyl, isoindolinyl, benzodioxolyl, dihydroisoindole, dihydrobenzofuranyl, dihydrobenzothiopyranyl, dihydroisoquinolinyl, dihydrobenzoxazinyl, dihydrobenzodioxazinyl, dihydrodioxolyl and dihydrochromenyl.

The term “heteroaryl” is intended to mean a 5-7 membered monocyclic aromatic or a fused 8-11 membered bicyclic aromatic ring, which monocyclic or bicyclic ring contains 1 to 3 heteroatoms selected from oxygen, nitrogen and sulphur. Suitable examples of such monocyclic aromatic rings include thienyl, furyl, pyrrolyl, triazolyl, imidazolyl, oxazolyl, thiazolyl, oxadiazolyl, isothiazolyl, isoxazolyl, thiadiazolyl, pyrazolyl, pyrimidyl, pyridazinyl, pyrazinyl, pyridyl and tetrahydropyranyl. Suitable examples of such fused aromatic rings include benzofused aromatic rings such as quinolinyl, isoquinolinyl, quinazolinyl, quinoxalinyl, cinnolinyl, naphthyridinyl, indolyl, indazolyl, furopyridinyl, pyrrolopyridinyl, benzofuranyl, benzothienyl, benzimidazolyl, benzoxazolyl, benzisoxazolyl, benzothiazolyl, benzisothiazolyl, benzoxadiazolyl, benzothiadiazolyl and the like. Suitable examples of such fused heteroaryl rings include thienopyridinyl, pyrazolopyrimidinyl, pyrazolopyridinyl, thienopyrazolyl and imidazothiazolyl.

In one aspect, R¹ represents —C₃₋₇ cycloalkyl (eg. cyclobutyl, cyclopentyl or cyclohexyl) optionally substituted by a C₁₋₃ alkyl (eg. methyl) group.

In a more particular aspect, R¹ represents unsubstituted cyclobutyl or cyclopentyl, especially unsubstituted cyclobutyl.

In one embodiment, R² represents
-aryl (eg. -phenyl) optionally substituted by one or more halogen (eg. fluorine), cyano, C₁₋₆ alkyl (eg. methyl)-CONR⁶R⁷ (eg. —CON(H)(Me)), C₁₋₆ alkylamidoC₁₋₆ alkyl (eg. —CH₂CON(H)(Me)) or —C₁₋₆ alkyl-COR⁵ (eg. —CH₂—COMe) groups;
-aryl-X-heteroaryl (eg. -phenyl-O-pyridinyl or -phenyl-CONH-pyridinyl) optionally substituted by one or more —CONR⁶R⁷ groups (eg. —CON(H)(Me));
-heteroaryl (eg. -pyridinyl, -thiazolyl or -furanyl) optionally substituted by one or more cyano, —CO₂R⁵ (eg. —CO₂H or —CO₂CH₃), —CONR⁶R⁷ (eg. —CON(H)(Me)) or alkylamidoalkyl (eg. CH₂CON(H)Me) groups;
-heteroaryl-X-heterocyclyl (eg. -pyridinyl-CO-morpholinyl);
-heterocyclyl (eg. piperazinyl, piperidinyl or oxazolidinyl) optionally substituted by one or more —SO₂NR⁶R⁷ (eg. —SO₂N(Me)₂), sulfonyl, haloC₁₋₆ alkyl (eg. —CH₂CF₃), C₁₋₆ alkylsulfonyl (eg. —SO₂Me or —SO₂CH(Me)₂), C₁₋₆ alkoxycarbonyl (eg. —COCH₂OCH(Me)₂), —COR⁵ (eg. —CO—CH₂—C(Me)₃), CO₂R⁵ (eg. —CO₂CH₂phenyl), ═O or hydroxyalkyl (eg. hydroxymethyl) groups;
-heterocyclyl-X—C₃₋₈ cycloalkyl (eg. -piperazinyl-CO-cyclopentyl, -piperazinyl-CO-cyclopropyl or -piperazinyl-CO-cyclohexyl) optionally substituted by one or more C₁₋₆ alkoxy (eg. —OC(CH₃)₃) groups;
-heterocyclyl-X-aryl (eg. -piperidinyl-CO-phenyl, -piperazinyl-phenyl, -piperazinyl-CO-phenyl, -piperazinyl-SO₂-phenyl, -piperazinyl-CO-naphthyl, -piperazinyl-SO₂-naphthyl, -piperazinyl-COCH₂-phenyl, -piperazinyl-COCH₂-naphthyl, -piperazinyl-COCH₂O-phenyl, -piperazinyl-CONH-phenyl, -piperazinyl-COCH₂NHCO-phenyl, -piperazinyl-SO₂CH₂-phenyl, -piperazinyl-SO₂(CH₂)₂-phenyl, -piperazinyl-SO₂(CH₂)₂-naphthyl, -piperazinyl-SO₂—CH═CH-phenyl, -piperazinyl-CO—CH═CH-phenyl, -piperazinyl-CO-dihydroindenyl, -piperazinyl-CO—C(H)(Me)-phenyl, -piperazinyl-CO—CH(NHCOCH₃)-phenyl, -piperazinyl-CO—CH(phenyl)-phenyl, -piperazinyl-CO—C(H)(Et)-CH₂-phenyl, -oxazolidinyl-CH₂O-phenyl, -piperidinyl-phenyl, -piperidinyl-CON H-phenyl, piperidinyl-CSNH-phenyl or -piperazinyl-CO-naphthyl) optionally substituted by one or more halogen (eg. chlorine, fluorine or bromine), hydroxy, cyano, nitro, ═O, C₁₋₆ alkyl (eg. methyl, ethyl, —CH(Me)₂ or —C(Me)₃), haloC₁₋₆ alkyl (eg. trifluoromethyl), C₁₋₆ alkoxy (eg. methoxy or —OCH(Me)₂), haloC₁₋₆ alkoxy (eg. trifluoromethoxy), —R⁵ (eg. phenyl, pyridinyl, furanyl, pyrazolyl or oxadiazolyl optionally substituted by one or more C₁₋₆ alkyl (eg. methyl) groups), —COR⁵ (eg. —CO-methyl, —CO-ethyl, —CO-trifluoromethyl, —CO-phenyl or —CO-piperidinyl), —CO₂R⁵ (eg. —COOH), aryloxy (eg. —O-phenyl), C₁₋₆ alkylsulfonyl (eg. —SO₂Me), —NR⁶R⁷ (eg. —N(Me)₂)—NR⁶COR⁷ (eg. —NHCOMe) groups;
-heterocyclyl-X-heterocyclyl (eg. -piperazinyl-CO-piperidinyl, -piperazinyl-CO-morpholinyl, -piperazinyl-CO-tetrahydropyranyl, -piperazinyl-CO-pyrrolidinyl, -piperazinyl-CO-dihydrochromenyl, -piperazinyl-SO₂-dihydrochromenyl, -piperazinyl-CO-dihydrobenzothiopyranyl, -piperazinyl-CO-dihydrobenzofuranyl, -piperazinyl-SO₂-dihydrobenzofuranyl, -piperazinyl-SO₂-dihydrobenzoxazinyl, -piperazinyl-SO₂-dihydrobenzodioxinyl, -piperazinyl-COCH₂-dihydroisoindolyl, -piperazinyl-COCH₂-dihydrobenzodioxolyl, -piperazinyl-COCH₂-piperidinyl, -piperidinyl-CO-tetrahydropyranyl or piperidinyl-CO-isoindolyl) optionally substituted by one or more C₁₋₆ alkyl (eg. methyl or —CH(Me)₂) or ═O groups; or
-heterocyclyl-X-heteroaryl (eg. -piperazinyl-CO-benzoxadiazolyl, -piperazinyl-SO₂-benzoxadiazolyl, -piperazinyl-CO-thiazolyl, -piperazinyl-COCH₂-thiazolyl, -piperazinyl-CO-thienyl, -piperazinyl-CONH-thienyl, -piperazinyl-COCH₂-thienyl, -piperazinyl-SO₂-thienyl, -piperazinyl-CO-quinolinyl, -piperazinyl-COCH₂-quinolinyl, -piperazinyl-SO₂-quinolinyl, -piperazinyl-CO-isoquinolinyl, -piperazinyl-SO₂-isoquinolinyl, -piperazinyl-CO-imidazolyl, -piperazinyl-COCH₂-imidazolyl, -piperazinyl-SO₂-imidazolyl, -piperazinyl-SO₂-thiazolyl, -piperazinyl-CO-pyrazolyl, -piperazinyl-SO₂-pyrazolyl, -piperazinyl-CO-benzothienyl, -piperazinyl-SO₂-benzothienyl, -piperazinyl-COCH₂-benzothienyl, -piperazinyl-SO₂-thienopyridinyl, -piperazinyl-CO-benzofuranyl, -piperazinyl-CO-oxadiazolyl, -piperazinyl-CO-indazolyl, -piperazinyl-CO-pyrazolopyrimidinyl, -piperazinyl-CO-oxazolyl, -piperazinyl-CO-thienopyrazolyl, -piperazinyl-CO-pyrazolopyridinyl, -piperazinyl-CO-benzothiazolyl, -piperazinyl-CO-furanyl, -piperazinyl-CO-indolyl, -piperazinyl-CO-pyridinyl, -piperazinyl-COCH₂-pyridinyl, -piperazinyl-SO₂-imidazothiazolyl, -piperazinyl-COCH₂-imidazothiazolyl, -piperazinyl-SO₂-isoxazolyl, -piperazinyl-CO-isoxazolyl, -piperazinyl-SO₂-pyridinyl, -piperazinyl-SO₂-pyridinyl or -piperazinyl-SO₂-benzothiadiazolyl, -piperidinyl-CO-pyridinyl, -piperidinyl-CO-pyrazinyl, -piperidinyl-CO-benzoxadiazolyl, -piperidinyl-CO-thiazolyl, -piperidinyl-pyridinyl, -piperidinyl-pyrazinyl, -piperidinyl-CONH-pyridinyl, piperidinyl-CO-quinoxalinyl or -piperidinyl-CO-pyrazolopyramidinyl) optionally substituted by one or more halogen (eg. chlorine), cyano, C₁₋₆ alkyl (eg. methyl), haloC₁₋₆alkyl (eg. —CF₃)═O, —R⁵ (eg. phenyl, isoxazolyl, oxazolyl or pyridinyl), —CO₂R⁵ (eg. —CO₂H, —CO₂CH₃ or —CO₂C(CH₃)₃), —NR⁶R⁷ (eg. pyrrolidinone). —CONR⁶R⁷ (eg. —CON(H)CH₃)) aryloxy (eg. —O-phenyl), —NR⁶COR⁷ (eg. —NHCOMe) or arylC₁₋₆ alkyl (eg. —CH₂-phenyl) groups.

In embodiments where R² is a substituted nitrogen containing heterocyclyl group, the nitrogen containing heterocyclyl group (eg. piperidinyl or piperazinyl) is typically substituted at the nitrogen atom.

Where R² represents -heterocyclyl-X-aryl, -heterocyclyl-X-heterocyclyl or -heterocyclyl-X-heteroaryl in which the heterocyclyl group attached to the tetrahydrobenzazepine contains one or more nitrogen atoms (e.g. piperidinyl or piperazinyl), the heterocyclyl group attached to the tetrahydrobenzazepine is typically linked to X through a nitrogen atom.

In a more particular embodiment, R² represents
-aryl-X-heteroaryl (eg. -phenyl-O-pyridinyl) optionally substituted by a —CONR⁶R⁷ group (eg. —CON(H)(Me)); or
-heterocyclyl-X-aryl (eg. -piperidinyl-CO-phenyl) optionally substituted by a cyano group.

In a most particular embodiment, R² represents
-heterocyclyl-X-aryl (eg. -piperidinyl-CO-phenyl) optionally substituted by a cyano group.

In one aspect, X represents a bond, O, CO, —CH₂O—, —COCH₂—, —COCH₂O—, —CONR^{2b} —(eg. —CONH—), —COCH₂NR^{2b} CO—(eg. —COCH₂NHCO—), SO₂, —SO₂C₁₋₃ alkyl- (eg. —SO₂—CH₂— or —SO₂—(CH₂)₂—), —SO₂C₂₋₃ alkenyl- (eg. —SO₂—CH═CH—), —COC₂₋₃ alkenyl- (eg. —CO—CH═CH—), —CO—C(R^{2a})(R^{2b})—(eg. —CO—C(H)(Me), —CO—C(H)(phenyl) or —CO—C(H)(NHCOMe)) or —CO—C(R^{2a})(R^{2b})CH₂— (eg. —CO—C(H)(Et)-CH₂—).

In a more particular aspect, X represents a bond, SO₂, CO or 0, most preferably CO.

In a further aspect, R^{2a} represents hydrogen and R^{2b} represents C₁₋₆ alkyl (eg. methyl or ethyl), aryl (eg. phenyl) or C₁₋₆ alkylamido (eg. —NHCOMe).

In another embodiment, R⁵ represents hydrogen, C₁₋₆ alkyl (eg. methyl, ethyl or —CH₂—C(Me)₃), haloC₁₋₆ alkyl (eg. trifluoromethyl), aryl (eg. phenyl), heterocyclyl (eg. piperidinyl), heteroaryl (eg. furanyl, pyridinyl, pyrazolyl, isoxazolyl, oxazolyl, oxadiazolyl) optionally substituted by one or more C₁₋₆ alkyl (eg. methyl) groups.

In a further aspect, R⁶ and R⁷ independently represent hydrogen or C₁₋₆ alkyl (eg. methyl).

In a further aspect, n represents 0 or 1, more preferably 0.

When n represents 1, R³ is preferably a halogen (eg. iodine) atom or a cyano group.

Compounds according to the invention include examples E1-E262 as shown below, or a pharmaceutically acceptable salt thereof.

One compound according to the invention includes 6-{[4-(3-cyclobutyl-2,3,4,5-tetrahydro-1H-3-benzazepin-7-yl)phenyl]oxy}-N-methyl-3-pyridinecarboxamide or a pharmaceutically acceptable salt thereof.

Another compound according to the invention is 4-{[4-(3-cyclobutyl-2,3,4,5-tetrahydro-1H-3-benzazepin-7-yl)-1-piperidinyl]carbonyl}benzonitrile or a pharmaceutically acceptable salt thereof.

Compounds of formula (I) may form acid addition salts with acids, such as conventional pharmaceutically acceptable acids, for example maleic, hydrochloric, hydrobromic, phosphoric, acetic, fumaric, salicylic, sulphate, citric, lactic, mandelic, tartaric and methanesulphonic. Salts, solvates and hydrates of compounds of formula (I) therefore form an aspect of the invention.

Certain compounds of formula (I) are capable of existing in stereoisomeric forms. It will be understood that the invention encompasses all geometric and optical isomers of these compounds and the mixtures thereof including racemates. Tautomers also form an aspect of the invention.

The present invention also provides a process for the preparation of a compound of formula (I) or a pharmaceutically acceptable salt thereof, which process comprises:
(a) reacting a compound of formula (II) 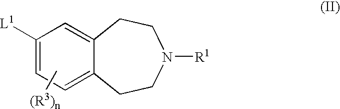
wherein R¹, R³ and n are as defined above and L¹ represents a suitable leaving group such as a halogen atom (eg. bromine or iodine), or an optionally activated hydroxyl group (such as a triflate group) with a compound of formula R²—Y, wherein R² is as defined above for R² and Y represents hydrogen or a suitable coupling group such as a boronic acid or organometallic group such as zinc or alkyl stannane; or
(b) reacting a compound of formula (III) 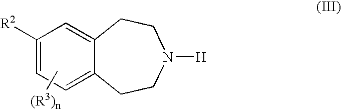
wherein R², R³ and n are as defined above, with a compound of formula R¹-L², wherein R¹ is as defined above and L² represents a suitable leaving group such as a halogen atom (eg. bromine, iodine or tosylate); or
(c) reacting a compound of formula (III) as defined above, with a ketone of formula R^{1′}═O, wherein R^{1′} is C₃₋₇ cycloalkyl optionally substituted by C₁₋₃ alkyl; or
(d) preparing a compound of formula (I) wherein R² represents -heterocyclyl, wherein said heterocyclyl is a 1,3-oxazolidin-2-one group substituted at the 5-position with a hydroxymethyl group, and wherein the oxazolidin-2-one group is attached to the benzazepine moiety through the nitrogen atom, which comprises reacting a compound of formula (IV) 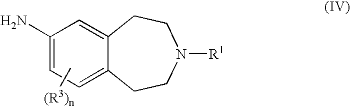
in a two step process, wherein R¹, R³ and n are as defined above, with a benzyl chloroformate group and then glycidol butyrate; or
(e) preparing a compound of formula (I) wherein R² represents -aryl, -heteroaryl, -aryl-X—C₃₋₈ cycloalkyl, -aryl-X-aryl, -aryl-X-heteroaryl, -aryl-X-heterocyclyl, -heteroaryl-X—C₃₋₈ cycloalkyl, -heteroaryl-X-aryl, -heteroaryl-X-heteroaryl, -heteroaryl-X-heterocyclyl, which comprises reacting a compound of formula (XI) 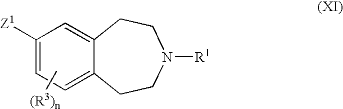
wherein R¹, R³ and n are as defined above and Z¹ represents a suitable coupling group such as a boronic acid or ester, or organometallic group such as zinc or alkyl stannane with a compound of formula R^{2″}-L¹, wherein L¹ represents a suitable leaving group such as a halogen atom (eg. bromine or iodine), or an optionally activated hydroxyl group (such as a triflate group) and R^{2″} represents the groups -aryl, -heteroaryl, -aryl-X—C₃₋₈ cycloalkyl, -aryl-X-aryl, -aryl-X-heteroaryl, -aryl-X-heterocyclyl, -heteroaryl-X—C₃₋₈ cycloalkyl, -heteroaryl-X-aryl, -heteroaryl-X-heteroaryl, -heteroaryl-X-heterocyclyl, or
(f) deprotecting a compound of formula (I) which is protected; or
(g) interconversion from another compound of formula (I).

When the compound of formula (II) represents an aryl electrophilic system, i.e. L¹ is a halogen atom (eg. bromine or iodine) or triflate group and R²—Y is a boronic acid (or ester), process (a) typically comprises the use of a palladium catalyst such as tetrakis(triphenylphosphine)palladium, in an appropriate solvent such as toluene or DME, with an appropriate base such as aqueous sodium carbonate at an appropriate temperature such as reflux.

When R²—Y is an amine, for example piperazine, process (a) typically comprises the use of a palladium catalyst such as palladium acetate, with an appropriate ligand such as o-biphenyl di-tert-butylphosphine in an appropriate solvent such as DME, with an appropriate base such as potassium phosphate, at an appropriate temperature such as reflux.

Process (b) typically comprises the use of a suitable base, such as potassium carbonate in an appropriate solvent such as 2-butanone optionally in the presence of a transfer reagent such as potassium iodide at an appropriate temperature such as reflux.

Process (c) typically comprises the use of reductive conditions (such as treatment with a borohydride eg. sodium triacetoxyborohydride), optionally in the presence of an acid, such as acetic acid, in an appropriate solvent such as dichloromethane at a suitable temperature such as room temperature.

Step 1 of process (d) typically comprises the use of a chloroformate such as benzyl chloroformate, with suitable base, such as sodium hydrogen carbonate in an appropriate solvent such as acetone. Step 2 of process (d) involves reacting the product of step 1 with glycidol butyrate according to WO 02/059115.

When the compound of formula (XI) represents an aryl boronic acid (or ester) and R²″-L¹ is an electrophilic aryl or heteroaryl system, i.e. L¹ is a halogen atom (eg. bromine or iodine) or triflate group, process (e) typically comprises the use of a palladium catalyst such as tetrakis(triphenylphosphine)palladium, in an appropriate solvent such as toluene, with an appropriate base such as aqueous sodium carbonate at an appropriate temperature such as reflux.

In process (f), examples of protecting groups and the means for their removal can be found in T. W. Greene ‘Protective Groups in Organic Synthesis’ (J. Wiley and Sons, 1991). Suitable amine protecting groups include sulphonyl (e.g. tosyl), acyl (e.g. acetyl, 2′,2′,2′-trichloroethoxycarbonyl, benzyloxycarbonyl or t-butoxycarbonyl) and arylalkyl (e.g. benzyl), which may be removed by hydrolysis (e.g. using an acid such as hydrochloric acid in dioxan or trifluoroacetic acid in dichloromethane) or reductively (e.g. hydrogenolysis of a benzyl group or reductive removal of a 2′,2′,2′-trichloroethoxycarbonyl group using zinc in acetic acid) as appropriate. Where the protecting group is benzyloxycarbonyl, this may be removed by hydrogenolysis using a suitable catalyst such as palladium on charcoal, at a suitable temperature (eg. room temperature) and at a suitable pressure of hydrogen (eg. atmospheric pressure) in a suitable solvent (eg. ethanol:methanol (1:1) or ethanol). Other suitable amine protecting groups include trifluoroacetyl (—COCF₃) which may be removed by base catalysed hydrolysis or a solid phase resin bound benzyl group, such as a Merrifield resin bound 2,6-dimethoxybenzyl group (Ellman linker), which may be removed by acid catalysed hydrolysis, for example with trifluoroacetic acid.

Process (g) may be performed using conventional interconversion procedures such as epimerisation, oxidation, reduction, alkylation, nucleophilic or electrophilic aromatic substitution, ester hydrolysis, amide bond formation or transition metal mediated coupling reactions. An example of a reduction reaction useful as an interconversion procedure would include the conversion of a heteroaryl group, such as a pyridyl group, to a heterocycyl group, for example a piperidyl group, using a catalyst system such as platinum oxide in the presence of hydrogen. Examples of transition metal mediated coupling reactions useful as interconversion procedures include the following: Palladium catalysed coupling reactions between organic electrophiles, such as aryl halides, and organometallic reagents, for example boronic acids (Suzuki cross-coupling reactions); Palladium catalysed amination and amidation reactions between organic electrophiles, such as aryl halides, and nucleophiles, such as amines and amides; Copper catalysed amidation reactions between organic electrophiles (such as aryl halides) and nucleophiles such as amides; and Copper mediated coupling reactions between phenols and boronic acids.

Compounds of formula (II) and (III) may be prepared in accordance with the following scheme: 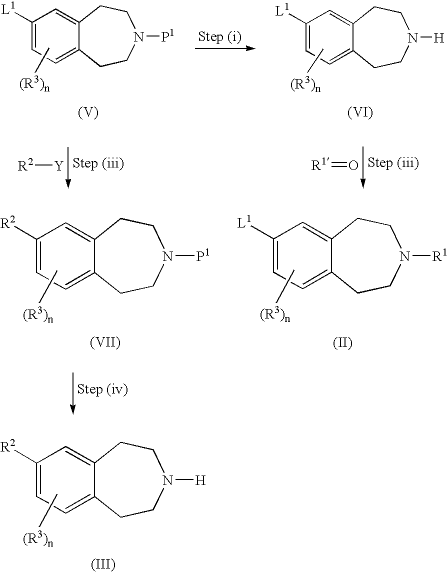
wherein R¹, R^{1′}, R², R³, n, Y and L¹ are as defined above and P¹ represents a suitable protecting group such as Boc.

Step (i) typically comprises a deprotection reaction, for example, when P¹ represents Boc the deprotection reaction comprises reaction of a compound of formula (V) with an acid, for example hydrochloric acid in dioxan or trifluoroacetic acid in dichloromethane.

Step (ii) may be performed under reducing conditions in an analogous manner to that described for process (c) above.

Step (iii) may be performed in an analogous manner to that described for process (a) above.

Step (iv) typically comprises a deprotection reaction to provide a compound of formula (III) and can be performed as described in step (i) above.

Compounds of formula (IV) may be prepared in accordance with the following scheme: 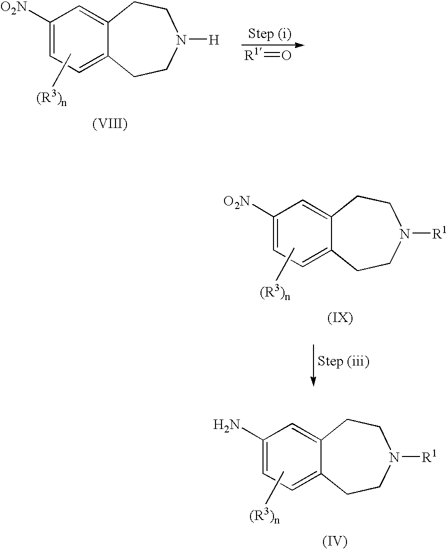
wherein R¹, R², R³ and n are as defined above.

Step (i) may be performed under reducing conditions in an analogous manner to that described for process (c) above. Alternatively, step (i) may be performed by reacting the compound of formula (VIII) with a compound of formula R¹-L², wherein R¹ is defined above and L² represents a suitable leaving group such as a halogen atom (eg. bromine, iodine or tosylate), in an analogous manner to that described for process (b) above.

Step (ii) typically comprises a hydrogenation reaction comprising 10% palladium on carbon paste in the presence of suitable solvents such as methanol and tetrahydrofuran.

Compounds of formula (VII) may also be prepared in accordance with the following scheme: 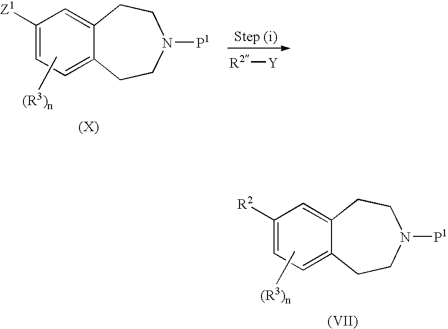
wherein R² represents -aryl, -heteroaryl, -aryl-X—C₃₋₈ cycloalkyl, -aryl-X-aryl, -aryl-X-heteroaryl, -aryl-X-heterocyclyl, -heteroaryl-X—C₃₋₈ cycloalkyl, -heteroaryl-X-aryl, -heteroaryl-X-heteroaryl, -heteroaryl-X-heterocyclyl, wherein R^{2″}, R³ and n are as defined above and wherein P¹ represents a suitable protecting group such as Boc and Z¹ represents a boronic ester or boronic acid or any other group suitable for transition metal mediated cross coupling reactions.

Step (i) may be performed with the use of a palladium catalyst such as tetrakis(triphenylphosphine)palladium, in an appropriate solvent such as toluene, with an appropriate base such as sodium carbonate at an appropriate temperature such as reflux.

Compounds of formula (XI) may be prepared in accordance with the following scheme: 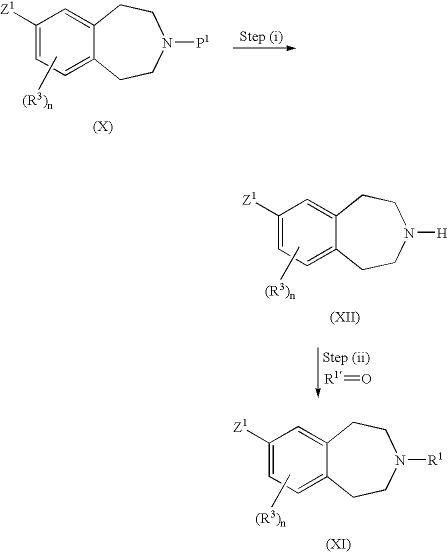
wherein R¹, R^{1′}, R³ and n are as defined above and P¹ represents a suitable protecting group such as Boc and Z¹ represents a boronic ester or boronic acid or any other group suitable for transition metal mediated cross coupling reactions.

Step (i) typically comprises a deprotection reaction, for example, when P¹ represents Boc the deprotection reaction comprises reaction of a compound of formula (V) with an acid, for example hydrochloric acid in dioxan or trifluoroacetic acid in dichloromethane.

Step (ii) may be performed under reducing conditions in an analogous manner to that described for process (c) above. Alternatively, step (ii) may be performed by reacting the compound of formula (XII) with a compound of formula R¹-L², wherein R¹ is defined above and L² represents a suitable leaving group such as a halogen atom (eg. bromine, iodine or tosylate), in an analogous manner to that described for process (b) above.

Compounds of formula (V) may be prepared in an analogous manner to those described in Description 3 of WO 02/040471.

Compounds of formula (VIII) may be prepared in an analogous manner to those described in WO 03/068752.

Compounds of formula (X) may be prepared in an analogous manner to those described in WO 2004056369 A1 (Example 264, step 1)

Compounds of formula (I) and their pharmaceutically acceptable salts have affinity for and are antagonists and/or inverse agonists of the histamine H3 receptor and are believed to be of potential use in the treatment of neurological diseases including Alzheimer's disease, dementia, age-related memory dysfunction, mild cognitive impairment, cognitive deficit, epilepsy, neuropathic pain, inflammatory pain, migraine, Parkinson's disease, multiple sclerosis, stroke and sleep disorders including narcolepsy; psychiatric disorders including schizophrenia (particularly cognitive deficit of schizophrenia), attention deficit hypereactivity disorder, depression and addiction; and other diseases including obesity, asthma, allergic rhinitis, nasal congestion, chronic obstructive pulmonary disease and gastro-intestinal disorders.

Thus the invention also provides a compound of formula (I) or a pharmaceutically acceptable salt thereof, for use as a therapeutic substance in the treatment or prophylaxis of the above disorders, in particular cognitive impairments in diseases such as Alzheimer's disease and related neurodegenerative disorders.

The invention further provides a method of treatment or prophylaxis of the above disorders, in mammals including humans, which comprises administering to the sufferer a therapeutically effective amount of a compound of formula (I) or a pharmaceutically acceptable salt thereof.

In another aspect, the invention provides the use of a compound of formula (I) or a pharmaceutically acceptable salt thereof in the manufacture of a medicament for use in the treatment of the above disorders.

When used in therapy, the compounds of formula (I) are usually formulated in a standard pharmaceutical composition. Such compositions can be prepared using standard procedures.

Thus, the present invention further provides a pharmaceutical composition for use in the treatment of the above disorders which comprises the compound of formula (I) or a pharmaceutically acceptable salt thereof and a pharmaceutically acceptable carrier.

The present invention further provides a pharmaceutical composition which comprises the compound of formula (I) or a pharmaceutically acceptable salt thereof and a pharmaceutically acceptable carrier.

Compounds of formula (I) may be used in combination with other therapeutic agents, for example histamine H1 antagonists or medicaments claimed to be useful as either disease modifying or symptomatic treatments of Alzheimer's disease. Suitable examples of such other therapeutic agents may be agents known to modify cholinergic transmission such as 5-HT₆ antagonists, M1 muscarinic agonists, M2 muscarinic antagonists or acetylcholinesterase inhibitors. When the compounds are used in combination with other therapeutic agents, the compounds may be administered either sequentially or simultaneously by any convenient route.

The invention thus provides, in a further aspect, a combination comprising a compound of formula (I) or a pharmaceutically acceptable derivative thereof together with a further therapeutic agent or agents.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations.

When a compound of formula (I) or a pharmaceutically acceptable derivative thereof is used in combination with a second therapeutic agent active against the same disease state the dose of each compound may differ from that when the compound is used alone. Appropriate doses will be readily appreciated by those skilled in the art.

A pharmaceutical composition of the invention, which may be prepared by admixture, suitably at ambient temperature and atmospheric pressure, is usually adapted for oral, parenteral or rectal administration and, as such, may be in the form of tablets, capsules, oral liquid preparations, powders, granules, lozenges, reconstitutable powders, injectable or infusible solutions or suspensions or suppositories. Orally administrable compositions are generally preferred.

Tablets and capsules for oral administration may be in unit dose form, and may contain conventional excipients, such as binding agents, fillers, tabletting lubricants, disintegrants and acceptable wetting agents. The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents, emulsifying agents, non-aqueous vehicles (which may include edible oils), preservatives, and, if desired, conventional flavourings or colorants.

For parenteral administration, fluid unit dosage forms are prepared utilising a compound of the invention or pharmaceutically acceptable salt thereof and a sterile vehicle. The compound, depending on the vehicle and concentration used, can be either suspended or dissolved in the vehicle. In preparing solutions, the compound can be dissolved for injection and filter sterilised before filling into a suitable vial or ampoule and sealing. Advantageously, adjuvants such as a local anaesthetic, preservatives and buffering agents are dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilisation cannot be accomplished by filtration.

The compound can be sterilised by exposure to ethylene oxide before suspension in a sterile vehicle. Advantageously, a surfactant or wetting agent is included in the composition to facilitate uniform distribution of the compound. The composition may contain from 0.1% to 99% by weight, preferably from 10 to 60% by weight, of the active material, depending on the method of administration. The dose of the compound used in the treatment of the aforementioned disorders will vary in the usual way with the seriousness of the disorders, the weight of the sufferer, and other similar factors. However, as a general guide suitable unit doses may be 0.05 to 1000 mg, more suitably 1.0 to 200 mg, and such unit doses may be administered more than once a day, for example two or three a day. Such therapy may extend for a number of weeks or months.

The following Descriptions and Examples illustrate the preparation of compounds of the invention.

Description 1

### 1,1-Dimethylethyl 7-{[(trifluoromethyl)sulfonyl]oxy}-1,2,4,5-tetrahydro-3H-3-benzazepine-3-carboxylate (D1)

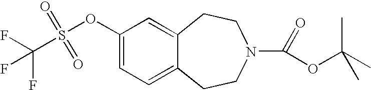

Trifluoroacetic anhydride (16 ml, 95 mmol) was added dropwise over 0.5 h to a solution of 1,1-dimethylethyl 7-hydroxy-1,2,4,5-tetrahydro-3H-3-benzazepine-3-carboxylate (PCT Int. Appl. (2003), 56 pp. CODEN: PIXXD2 WO 2003068752 A1; 25 g, 94.93 mmol) and triethylamine (20 ml, 142 mmol) in dry dichloromethane (250 ml) at −25° C. The reaction mixture was allowed to warm to room temperature and stirred for 16 hours. Saturated sodium bicarbonate solution (250 ml) was cautiously added and the mixture vigorously stirred for 10 minutes. The aqueous phase was separated and re-extracted with dichloromethane (2×100 ml). The combined organic extracts were washed with citric acid (3M; 2×200 ml), followed by saturated sodium bicarbonate (2×100 ml), then brine (200 ml) and dried over anhydrous sodium sulfate in the presence of activated charcoal, filtered and evaporated. The crude material was purified by chromatography on silica, eluting with a mixture of ethyl acetate:pentane 1:10 to 1:5 to give the title product MS (ES+) m/e 396 [M+H]⁺.

Description 2

### 1,1-Dimethylethyl 7-(4-{[(phenylmethyl)oxy]carbonyl}-1-piperazinyl)-1,2,4,5-tetrahydro-3H-3-benzazepine-3-carboxylate (D2)

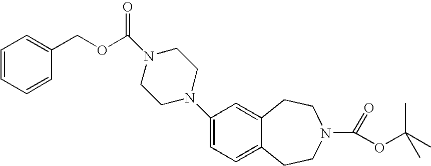

1,1-Dimethylethyl 7-{[(trifluoromethyl)sulfonyl]oxy}-1,2,4,5-tetrahydro-3H-3-benzazepine-3-carboxylate (D1) (36 g, 91 mmol) was added to a solution of palladium acetate (1.5 g, 6.6 mmol), o-biphenyl di-tert-butylphosphine (4 g, 13.6 mmol) and potassium phosphate (tribasic; 29 g, 136.5 mmol) in dry DME (1 litre). The mixture was purged with argon for 30 minutes then phenylmethyl 1-piperazinecarboxylate (Aldrich, 45, 692-6; 26 g, 118 mmol) was added and the mixture stirred at 80° C. under argon for 5 hours. The mixture was cooled to room temperature and diethyl ether (1 litre) was added. The mixture was filtered through celite and the filtrate evaporated. The residue was purified by chromatography on silica, eluting with a mixture of ethyl acetate:pentane 1:3 to give the title product MS (ES+) m/e 466 [M+H]⁺.

Description 3

### Phenylmethyl 4-(2,3,4,5-tetrahydro-1H-3-benzazepin -7-yl)-1-piperazinecarboxylate (D3)

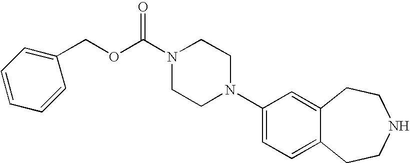

Trifluoroacetic acid (100 ml; 1.33 mol) was added dropwise over 30 minutes to a solution of 1,1-dimethylethyl 7-(4-{[(phenylmethyl)oxy]carbonyl}-1-piperazinyl)-1,2,4,5-tetrahydro-3H-3-benzazepine-3-carboxylate (D2) (24.8 g, 53.3 mmol) in dichloromethane (300 ml) at 0° C. under argon. The mixture was stirred for 6 hours, the solvent was then evaporated to dryness and the residue purified by chromatography on silica, eluting with a mixture 0.880 ammonia: methanol: dichloromethane (1:9:90) to afford the title product; MS (ES+) m/e 366 [M+H]⁺.

Description 4

### Phenylmethyl 4-(3-cyclobutyl-2,3,4,5-tetrahydro-1H-3-benzazepin-7-yl)-1-piperazinecarboxylate (D4)

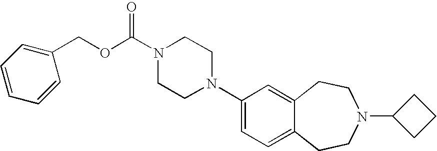

Cyclobutanone (287 mg, 4.1 mmol) was added to a solution of phenylmethyl 4-(2,3,4,5-tetrahydro-1H-3-benzazepin-7-yl)-1-piperazinecarboxylate (D3) (1 g, 2.7 mmol) in dichloromethane (15 ml) containing glacial acetic acid (2.5%). The mixture was stirred for 1 hour at room temperature, then sodium triacetoxyborohydride (870 mg, 4.1 mmol) was added and the mixture stirred at room temperature for 4 hours. The reaction mixture was partitioned between sodium carbonate (2M, 200 ml) and dichloromethane (2×200 ml). The combined organic extracts were washed with brine (200 ml), dried over sodium sulphate, filtered and evaporated. The residue was purified by chromatography on silica, eluting with a mixture 0.880 ammonia: methanol: dichloromethane (0.5:4.5:95) to afford the title product; MS (ES+) m/e 420 [M+H]⁺.

Description 5

### Phenylmethyl 4-(3-cyclopentyl-2,3,4,5-tetrahydro-1H-3-benzazepin-7-yl)-1-piperazinecarboxylate (D5)

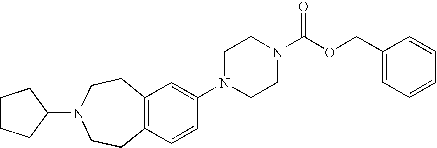

1,1-Dimethylethyl 7-(4-{[(phenylmethyl)oxy]carbonyl}-1-piperazinyl)-1,2,4,5-tetrahydro-3H-3-benzazepine-3-carboxylate (D2) (4.2 g, 9.1 mmol) was dissolved in dichloromethane (10 ml) and cooled to 0° C. before the slow addition of trifluoroacetic acid (7.0 ml, 90 mmol). The solution was stirred at room temperature for 3 hours and concentrated in vacuo. The crude residue was partitioned between dichloromethane and a 10% sodium bicarbonate solution (pH=11). The organic solution was concentrated in vacuo and dried for 1 hour (1 mbar, 20° C.). To the dry residue dissolved in dichloromethane (50 ml), cyclopentanone (1.61 ml, 18.2 mmol) and acetic acid (0.52 ml, 9.1 mmol) were added and the solution was stirred for 1 hour before the addition of sodium triacetoxyborohydride (3.86 g, 18.2 mmol). The reaction was stirred at room temperature for 2 days. A 2N hydrochloric acid aqueous solution (4.5 ml, 9.1 mmol) was added slowly at 0° C. followed by the slow addition of a 3N sodium hydroxide aqueous solution until pH ˜9. The aqueous phase was extracted 3 times with dichloromethane. The combined extracts were washed with water, brine, dried over magnesium sulfate and concentrated in vacuo. The title product was purified by column chromatography eluting with a mixture of dichloromethane:methanol (95:5); MS (ES+) m/e 434 [M+H]⁺; ¹H NMR (CDCl₃) 7.37-7.29 (5H, m), 6.98 (1H, m), 6.69-6.64 (2H, m), 5.16 (2H, s), 3.66-3.63 (4H, m), 3.10 (4H, brs), 2.91-2.85 (5H, m), 2.72-2.70 (4H, brs), 1.86-1.82 (2H, m), 1.67 (2H, m), 1.55-1.45 (4H, m).

Description 6

### 3-Cyclopentyl-7-(1-piperazinyl)-2,3,4,5-tetrahydro-1H-3-benzazepine (D6)

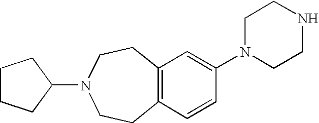

Phenylmethyl 4-(3-cyclopentyl-2,3,4,5-tetrahydro-1H-3-benzazepin-7-yl)-1-piperazinecarboxylate (D5) (2.29 g, 5.3 mmol) was dissolved in a mixture of ethanol:methanol (1:1) (100 ml). Palladium (0.5 g, 10% on charcoal paste) was added and the reaction mixture was stirred at room temperature under hydrogen (atmospheric pressure) for 12 hours. The mixture was filtered through celite and the filtrate concentrated in vacuo and dried overnight (1 mbar, 20° C.) to afford the title product; MS (ES+) m/e 300 [M+H]+; ¹H NMR (CDCl₃) 6.98 (1H, m), 6.70-6.65 (2H, m), 3.73-3.67 (1H, brs), 3.14-3.11 (4H, m), 3.05-3.03 (4H, m), 2.95-2.91 (5H, m), 2.81-2.78 (4H, brs), 1.91-1.88 (2H, m), 1.71-1.67 (2H, m), 1.58-1.52 (4H, m).

Description 7

### 3-Cyclobutyl-7-(1-piperazinyl)-2,3,4,5-tetrahydro-1H-3-benzazepine (D7)

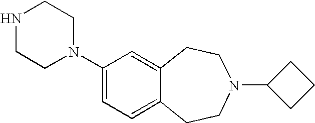

The title compound was prepared from phenylmethyl 4-(3-cyclobutyl-2,3,4,5-tetrahydro-1H-3-benzazepin-7-yl)-1-piperazinecarboxylate (D4) using an analogous method to that described for Description 6; MS (ES+) m/e 286 [M+H]⁺

Description 8

### 4-Bromo-3-fluoro-N-methylbenzamide (D8)

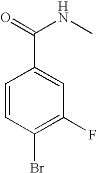

A mixture of 4-bromo-3-fluorobenzoic acid (470 mg, 2.14 mmol), methylamine (2M in tetrahydrofuran, 4.3 ml, 4.3 mmol), polymer bound dicyclohexylcarbodiimide resin (2.5 g, 4.3 mmol, 1.7 mmol/g), 1-hydroxybenzotriazole (580 mg, 4.3 mmol) and dichloromethane (15 ml) were stirred at room temperature for 48 hours. The reaction mixture was filtered and solvent was removed in vacuo. The product was dissolved in methanol and applied to a SCX cartridge (Varian bond-elute, 10 g) and washed with methanol and then a mixture of 2M ammonia/methanol. The product was purified further by column chromatography eluting 0.5% (2M ammonia in methanol/dichloromethane) to afford the title compound. MS (ES+) m/e 233 [M+H]⁺.

Description 9

### 1,1-Dimethylethyl 7-(1-{[(phenylmethyl)oxy]carbonyl}-1,2,3,6-tetrahydro-4-pyridinyl)-1,2,4,5-tetrahydro-3H-3-benzazepine-3-carboxylate (D9)

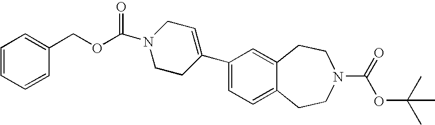

A mixture of phenylmethyl 4-(4,4,5,5-tetramethyl-1,3,2-dioxaborolan-2-yl)-3,6-dihydro-1(2H)-pyridinecarboxylate (Tetrahedron Letters 41(2000), 3705) (550 mg, 1.6 mmol), [1,1′-bis(diphenylphosphino)ferrocene]dichloropalladium(II) (67 mg, 10 mmol) and potassium carbonate (630 mg, 4.6 mmol) were suspended in degassed N,N-dimethylformamide (7 ml). 1,1-dimethylethyl 7-{[(trifluoromethyl)sulfonyl]oxy}-1,2,4,5-tetrahydro-3H-3-benzazepine-3-carboxylate (D1) (PCT Int. Appl. (2002), WO 2002040471 A2) (601 mg, 1.5 mmol) was then added and the mixture heated at 80° C. overnight. The crude mixture was filtered through a pad of celite and washed with ethyl acetate. The filtrate was washed with water, brine, dried over anhydrous magnesium sulfate, filtered and concentrated. The residue was purified by flash chromatography, eluting with a gradient of pentane to 20% ethyl acetate in pentane, to afford the title compound. MS (ES+) m/e 463 [M+H]⁺.

Description 10

### Phenylmethyl 4-(2,3,4,5-tetrahydro-1H-3-benzazepin-7-yl)-3,6-dihydro-1 (2H)-pyridinecarboxylate (D10)

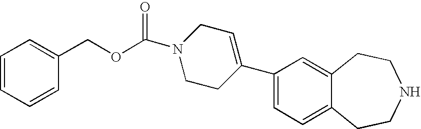

1,1-Dimethylethyl 7-(1-{[(phenylmethyl)oxy]carbonyl}-1,2,3,6-tetrahydro-4-pyridinyl)-1,2,4,5-tetrahydro-3H-3-benzazepine-3-carboxylate (D9) (480 mg, 0.97 mmol) was dissolved in dichloromethane (3 ml) at 0° C. and treated with trifluoroacetic acid (3 ml). The solution was stirred at room temperature for 1 hour and concentrated in vacuo, co-evaporating with dichloromethane. The residue was applied to a SCX cartridge (Varian Bond-elute, 10 g) and washed with methanol, then 2M ammonia in methanol. The product containing fractions were concentrated in vacuo to a solid that was used in subsequent steps without further purification. MS (ES+) m/e 363 [M+H]⁺.

Description 11

### Phenylmethyl 4-(3-cyclobutyl-2,3,4,5-tetrahydro-1H-3-benzazepin-7-yl)-3,6-dihydro-1(2H)-pyridinecarboxylate (D11)

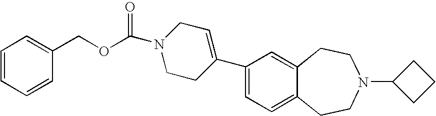

A solution of phenylmethyl 4-(2,3,4,5-tetrahydro-1H-3-benzazepin-7-yl)-3,6-dihydro-1 (2H)-pyridinecarboxylate (D10) (280 mg, 0.8 mmol) and cyclobutanone (0.12 ml, 1.5 mmol), were stirred at room temperature for 30 minutes in 2.5% acetic acid in methanol. Polystyryl(methyl)trimethylammonium cyanoborohydride (375 mg, 4 mmol/g loading, 1.5 mmol) was added and the solution stirred at room temperature overnight. The reaction mixture was loaded directly on to SCX (Varian Bond-elute, 10 g) washing with methanol and eluting product with 2M ammonia in methanol. Product containing fractions were concentrated in vacuo and the residue purified by flash chromatography, eluting with a gradient of dichloromethane to 1:9:90.880 ammonia:ethanol:dichloromethane to give the title compound MS (ES+) m/e 417 [M+H]⁺.

Description 12

### 3-Cyclobutyl-7-(4-piperidinyl)-2,3,4,5-tetrahydro-1H-3-benzazepine (D12)

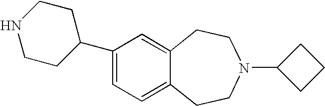

A solution of phenylmethyl 4-(3-cyclobutyl-2,3,4,5-tetrahydro-1H-3-benzazepin-7-yl)-3,6-dihydro-1 (2H)-pyridinecarboxylate (D11) (150 mg, 0.36 mmol) in ethanol (10 ml) was hydrogenated at atmospheric pressure over 10% palladium/charcoal (50 mg) for 48 hours. The catalyst was filtered, washed with ethanol and the filtrate concentrated in vacuo to afford the title product that was used in the subsequent step without further purification. MS (ES+) m/e 285 [M+H]⁺.

Description 13

### 4-Iodo-N-3-pyridinylbenzamide (D13)

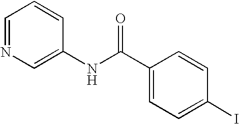

3-Aminopyridine (0.12 g, 1.3 mmol) and triethylamine (0.31 ml, 2.3 mmol) in dichloromethane (10 ml) were cooled to 0° C. and treated with 4-iodobenzoyl chloride (0.25 g, 0.94 mmol). The reaction mixture was stirred at room temperature for 2 hours, after which it was diluted with dichloromethane, washed with water, dried (MgSO₄) and concentrated in vacuo. The residue was triturated in dichloromethane (5 ml) and filtered to afford the title compound; MS (ES+), m/e 325 [M+H]⁺

### EXAMPLE 1

### 3-[4-(3-Cyclobutyl-2,3,4,5-tetrahydro-1H-3-benzazepin-7-yl)-1-piperazinyl]benzonitrile (E1)

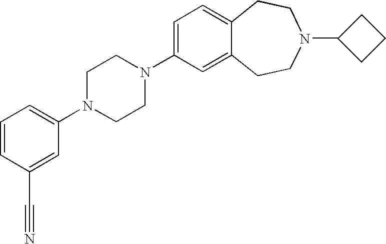

3-Cyclobutyl-7-(1-piperazinyl)-2,3,4,5-tetrahydro-1H-3-benzazepine (D7) (111.3 mg, 0.39 mmol), 3-bromobenzonitrile (70.6 mg, 0.39 mmol), cesium carbonate (178 mg, 0.55 mmol), palladium acetate (4 mg, 0.018 mmol) and (9,9-dimethyl-9H-xanthene-4,5-diyl)bis(diphenylphosphane) (15 mg, 0.027 mmol) were mired in 2 ml of dry toluene. The reaction mixture was heated in microwave at 140° C. for 25 minutes. Ethyl acetate was added and the mixture filtered through celite, washed with water and brine, dried over anhydrous sodium sulfate and concentrated in vacuo. The crude residue was purified by column chromatography eluting with a mixture of 0.880 ammonia:methanol:dichloromethane (0.5:4.5:95) to afford the title product; MS (ES+) m/e 387 [M+H]⁺.

### EXAMPLES 2-3 (E2-3)

Examples 2-3 were prepared from 3-cyclobutyl-7-(1-piperazinyl)-2,3,4,5-tetrahydro-1H-3-benzazepine (D7) and the appropriate benzonitrile using the analogous method to that described for Example 1 (see table)

| | | Heating | LC/MS |
|---|---|---|---|
| Example | Benzonitrile | time | (M + H⁺) |
| 4-[4-(3-Cyclobutyl- | 4- | 30 mins | 387 |
| 2,3,4,5-tetrahydro-1H-3- | bromobenzonitrile | | |
| benzazepin-7-yl)-1- | | | |
| piperazinyl]benzonitrile (E2) | | | |
| 2-[4-(3-Cyclobutyl- | 2- | 100 mins | 387 |
| 2,3,4,5-tetrahydro-1H-3- | bromobenzonitrile | | |
| benzazepin-7-yl)-1- | | | |
| piperazinyl]benzonitrile (E3) | | | |

### EXAMPLE 4

### 7-{4-[(2-Chlorophenyl)carbonyl]-1-piperazinyl}-3-cyclobutyl-2,3,4,5-tetrahydro-1H-3-benzazepine (E4)

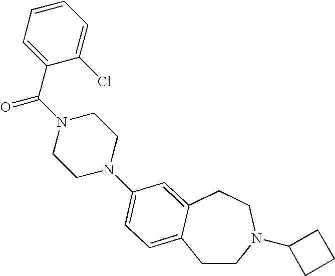

A mixture of 2-chlorophenylbenzoic acid (75 mg, 0.48 mmol), 1H-1,2,3-benzotriazol-1-ol (65 mg, 0.48 mmol) and N-cyclohexylcarbodiimide, N′-methyl polystyrene (1.8 mmol/g) (470 mg, 0.8 mmol) were stirred at room temperature in dichloromethane for 20 minutes. 3-cyclobutyl-7-(1-piperazinyl)-2,3,4,5-tetrahydro-1H-3-benzazepine (D7) (114 mg, 0.4 mmol) was added and the mixture stirred at room temperature overnight. The reaction mixture was loaded directly on to a SCX (Varian Bond-elute, 5 g) washing with methanol and eluting basic components with 2M ammonia in methanol. The product containing fractions were concentrated in vacuo and purified by flash chromatography eluting with a gradient of dichloromethane to 10% 2M ammonia in methanol, to afford the title product. MS (ES+) m/e 424 [M+H]⁺.

### EXAMPLE 5

### 3-Cyclobutyl-7-[4-(tetrahydro-2H-pyran-4-ylcarbonyl)-1-piperazinyl]-2,3,4,5-tetrahydro-1H-3-benzazepine (E5)

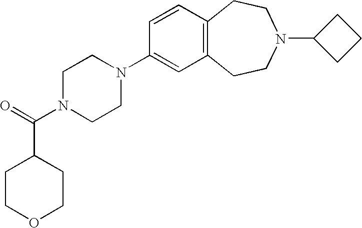

Example 5 was prepared in an analogous manner to Example 4 using tetrahydro-2H-pyran-4-carboxylic acid. MS (ES+) m/e 398 [M+H]⁺.

### EXAMPLE 6

### 4-{[4-(3-Cyclobutyl-2,3,4,5-tetrahydro-1H-3-benzazepin-7-yl)-1-piperazinyl]carbonyl}benzonitrile (E6)

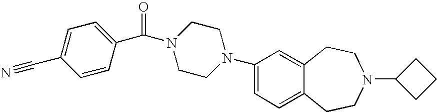

A mixture 3-cyclobutyl-7-(1-piperazinyl)-2,3,4,5-tetrahydro-1H-3-benzazepine (D7) (57 mg, 0.2 mmol) and polymer bound triethylamine (3.2 mmol/g; 625 mg, 2 mmol) were suspended in dichloromethane (5 ml). The mixture was treated with 4-cyanobenzoyl chloride (80 mg, 0.48 mmol) and stirred at room temperature overnight. The resin was filtered and the filtrate concentrated in vacuo. The residue was purified by flash chromatography, eluting with a gradient of dichloromethane to 1:9:90.880 ammonia:ethanol:dichloromethane, to afford the title compound. MS (ES+) m/e 415 [M+H]⁺.

### EXAMPLES 7-9 (E7-9)

The following examples were prepared in an analogous manner to Example 6 using 3-cyclobutyl-7-(1-piperazinyl)-2,3,4,5-tetrahydro-1H-3-benzazepine (D7) and the appropriate sulfonyl or acid chloride.

| | Sulfonyl/Acid | LC/MS |
|---|---|---|
| Example | chloride | (M + H⁺) |
| 7-[4-(2,1,3-Benzoxadiazol-5-ylcarbonyl)-1- | 2,1,3-benzoxadiazole- | 432 |
| piperazinyl]-3-cyclobutyl-2,3,4,5-tetrahydro-1H-3- | 5-carbonyl chloride | |
| benzazepine (E7) | | |
| 7-{4-[(2-Chlorophenyl)sulfonyl]-1-piperazinyl}-3- | 2-chlorobenzene | 461 |
| cyclobutyl-2,3,4,5-tetrahydro-1H-3-benzazepine | sulfonyl chloride | |
| (E8) | | |
| 3-Cyclobutyl-7-[4-(4-morpholinylcarbonyl)-1- | 4-morpholinecarbonyl | 399 |
| piperazinyl]-2,3,4,5-tetrahydro-1H-3-benzazepine | chloride | |
| (E9) | | |

### EXAMPLE 10

### 4-(3-Cyclobutyl-2,3,4,5-tetrahydro-1H-3-benzazepin-7-yl)-N-phenyl-1-piperazinecarboxamide (E10)

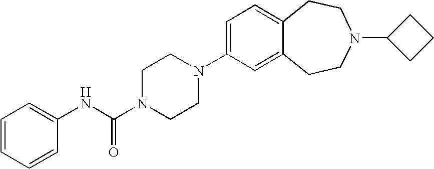

In a solution of dry tetrahydrofuran (5 ml) and diisopropylethylamine (0.2 ml, 1.14 mmol) cooled at −10° C., triphosgene (67.5 mg; 0.23 mmol) was added. After 5 minutes stirring at −10° C., a solution of 3-cyclobutyl-7-(1-piperazinyl)-2,3,4,5-tetrahydro-1H-3-benzazepine (D7) (130 mg, 0.46 mmol) in dry tetrahydrofuran (3 ml) and diisopropylethylamine (0.2 ml, 1.14 mmol) was added dropwise and stirred at room temperature for 30 minutes. After this time aniline was slowly added with dry tetrahydrofuran (4 ml) Reaction mixture was left to stir under argon at room temperature overnight. The mixture was acidified with acetic acid and applied to a SCX ion exchange cartridge (Varian bond-elute, 10 g) and washed with methanol followed by a mixture of 0.880 ammonia:methanol (1:9). The combined basic fractions were concentrated in vacuo and the resulting residue was purified by column chromatography eluting with a mixture of 0.880 ammonia: methanol: dichloromethane (0.25:2.25:97.5) to afford the title product; MS (ES+) m/e 405 [M+H]⁺.

### EXAMPLES 11-14 (E11-14)

Examples 11-14 were prepared from 3-cyclobutyl-7-(1-piperazinyl)-2,3,4,5-tetrahydro-1H-3-benzazepine (D7) and the appropriate aniline indicated in the table, using an analogous method to that described for Example 10.

| | | LC/MS |
|---|---|---|
| Product | Aniline | (M + H+) |
| 4-(3-Cyclobutyl-2,3,4,5-tetrahydro-1H-3-benzazepin-7-yl)- | 4- | 435 |
| N-[4-(methyloxy)phenyl]-1-piperazinecarboxamide (E11) | (methyloxy)aniline | |
| 4-(3-Cyclobutyl-2,3,4,5-tetrahydro-1H-3-benzazepin-7-yl)- | 3- | 435 |
| N-[3-(methyloxy)phenyl]-1-piperazinecarboxamide (E12) | (methyloxy)aniline | |
| N-(4-Chlorophenyl)-4-(3-cyclobutyl-2,3,4,5-tetrahydro-1H- | 4-chloroaniline | 439 |
| 3-benzazepin-7-yl)-1-piperazinecarboxamide (E13) | | |
| 4-(3-Cyclobutyl-2,3,4,5-tetrahydro-1H-3-benzazepin-7-yl)- | 4-ethylaniline | 433 |
| N-(4-ethylphenyl)-1-piperazinecarboxamide (E14) | | |

### EXAMPLE 15

### 1-(4-{[4-(3-Cyclobutyl-2,3,4,5-tetrahydro-1H-3-benzazepin-7-yl)-1-piperazinyl]carbonyl}phenyl)-2,2,2-trifluoroethanone (E15)

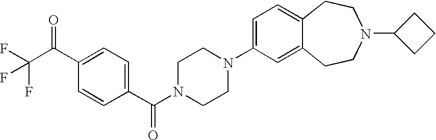

A mixture of 4-(trifluoroacetyl)benzoic acid (105 mg, 0.48 mmol), N-Cyclohexylcarbodiimide N′-methyl polystyrene (565 mg, 0.96 mmol), and 1-hydroxybenzotriazole (129 mg, 0.96 mmol) in dry dimethylformamide (5 ml) were stirred under argon at room temperature for 30 minutes. A solution of 3-cyclobutyl-7-(1-piperazinyl)-2,3,4,5-tetrahydro-1H-3-benzazepine (D7) (114 mg, 0.4 mmol) in dry dimethylformamide (1 ml) was added, and the reaction mixture left to stir at room temperature for one day. The mixture was applied to a SCX ion exchange cartridge (Varian bond-elute, 10 g), washed with methanol followed by a mixture of 0.880 ammonia:methanol (1:9). The combined basic fractions were concentrated in vacuo and the resulting residue purified by column chromatography eluting with a mixture of 0.880 ammonia: methanol: dichloromethane (0.25:2.25:97.5) to afford the title product; MS (ES+) m/e 486 [M+H]⁺.

### EXAMPLE 16

### 1-(4-{[4-(3-Cyclobutyl-2,3,4,5-tetrahydro-1H-3-benzazepin-7-yl)-1-piperazinyl]carbonyl}phenyl)-1-propanone (E16)

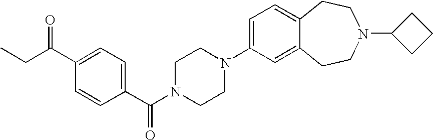

The title compound was prepared from 4-propanoylbenzoic acid and 3-cyclobutyl-7-(1-piperazinyl)-2,3,4,5-tetrahydro-1H-3-benzazepine (D7) using the same method described for the preparation of Example 15; MS (ES+) m/e 446 [M+H]⁺.

### EXAMPLE 17

### 3-Cyclobutyl-7-(4-{[4-(3-pyridinyl)phenyl]carbonyl}-1-piperazinyl)-2,3,4,5-tetrahydro-1H-3-benzazepine (E17)

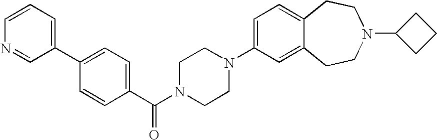

A mixture of o-benzotriazol-1-yl-N,N,N′,N′-tetramethyluroniumhexafluorophosphate (173 mg, 0.46 mmol) and 4-(3-pyridinyl)benzoic acid (91.6 mg, 0.46 mmol) in dry dimethylformamide (5 ml) was stirred for 30 minutes at room temperature. A solution of 3-cyclobutyl-7-(1-piperazinyl)-2,3,4,5-tetrahydro-1H-3-benzazepine (D7) (108.5 mg, 0.38 mmol) in dry dimethylformamide (5 ml) was then added followed by morpholinomethyl polystyrene HL (265 mg, 1.14 mmol). The reaction mixture was stirred at room temperature under argon overnight, then applied to a SCX ion exchange cartridge (Varian bond-elute, 10 g) and washed with methanol followed by a mixture of 0.880 ammonia:methanol (1:9). The combined basic fractions were concentrated in vacuo and the resulting residue was purified by column chromatography eluting with a mixture of 0.880 ammonia: methanol:dichloromethane (0.5:4.5:95) to afford the title product; MS (ES+) m/e 467 [M+H]⁺.

### EXAMPLE 18

### 3-Cyclobutyl-7-{4-[(2-methyl-5-phenyl-1,3-thiazol-4-yl)carbonyl]-1-piperazinyl}-2,3,4,5-tetrahydro-1H-3-benzazepine (E18)

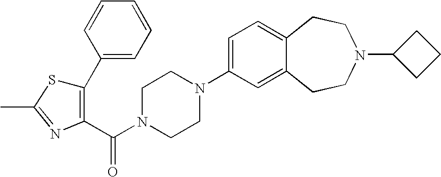

The title compound was prepared from 2-methyl-5-phenyl-1,3-thiazole-4-carboxylic acid (U.S. (1966), 5 pp. CODEN: USXXAM US 3282927) and 3-cyclobutyl-7-(1-piperazinyl)-2,3,4,5-tetrahydro-1H-3-benzazepine (D7) using the same method described for the preparation of Example 11. MS (ES+) m/e 487 [M+H]⁺.

### EXAMPLE 19

### 3-Cyclopentyl-7-{4-[(3,4-dichlorophenyl)sulfonyl]-1-piperazinyl}-2,3,4,5-tetrahydro-1H-3-benzazepine (E19)

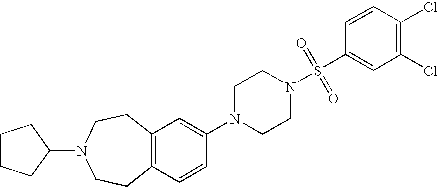

3-Cyclopentyl-7-(1-piperazinyl)-2,3,4,5-tetrahydro-1H-3-benzazepine (D6) (0.02 g, 0.07 mmol) was dissolved in dichloromethane (0.5 ml) before 3,4-dichlorobenzenesulfonyl chloride (0.013 ml, 0.08 mmol) was added followed by morpholinomethyl polystyrene resin (4.3 mmol/g, 50 mg, 0.22 mmol). The reaction mixture was shaken for 12 hours at room temperature. Scavenging MP-isocyanate resin (3 mmol/g, 50 mg) and Argopore-Trisamine resin (2.50 mmol/g, 50 mg) were added and the mixture was shaken for 1 day. Resins were filtered and washed with dichloromethane and the filtrate concentrated in vacuo to afford the title compound; MS (ES+) m/e 508 [M+H]⁺; ¹H NMR (CDCl₃) 7.87 (1H, s), 7.64-7.59 (2H, m), 6.98 (1H, d), 6.63-6.60 (2H, m), 3.20 (6H, brs), 2.88 (5H, m), 2.71 (4H, brs), 1.86 (4H, m), 1.68 (2H, m), 1.53 (4H, m).

### EXAMPLES 20-90

Examples 20-90 (E20-90) were prepared from 3-cyclopentyl-7-(1-piperazinyl)-2,3,4,5-tetrahydro-1H-3-benzazepine (D6) and the appropriate sulfonyl chloride indicated in the table using an analogous method to that described for Example 19 (E19).

| | | LC/MS |
|---|---|---|
| Example | Sulfonyl chloride | (M + H)⁺ |
| 3-Cyclopentyl-7-[4-(2-thienylsulfonyl)-1-piperazinyl]- | 2-thiophene | 446 |
| 2,3,4,5-tetrahydro-1H-3-benzazepine (E20) | sulfonyl chloride | |
| 4-{[4-(3-Cyclopentyl-2,3,4,5-tetrahydro-1H-3- | 4-cyanobenzene | 465 |
| benzazepin-7-yl)-1-piperazinyl]sulfonyl}benzonitrile | sulfonyl chloride | |
| (E21) | | |
| 3-Cyclopentyl-7-{4-[(4-methyl-3,4-dihydro-2H-1,4- | 4-methyl-3,4- | 511 |
| benzoxazin-7-yl)sulfonyl]-1-piperazinyl}-2,3,4,5- | dihydro-2H-1,4- | |
| tetrahydro-1H-3-benzazepine (E22) | benzoxazine-7- | |
| | sulfonyl chloride | |
| 3-Cyclopentyl-7-(4-{[4-(phenyloxy)phenyl]sulfonyl}-1- | 4-(phenyloxy) | 532 |
| piperazinyl)-2,3,4,5-tetrahydro-1H-3-benzazepine | benzenesulfonyl | |
| (E23) | chloride | |
| 3-Cyclopentyl-7-[4-(2,3-dihydro-1,4-benzodioxin-6- | 2,3-dihydro-1,4- | 498 |
| ylsulfonyl)-1-piperazinyl]-2,3,4,5-tetrahydro-1H-3- | benzodioxin-6- | |
| benzazepine (E24) | sulfonyl chloride | |
| 7-(4-{[3,4-Bis(methyloxy)phenyl]sulfonyl}-1- | 3,4-bis(methyloxy) | 500 |
| piperazinyl)-3-cyclopentyl-2,3,4,5-tetrahydro-1H-3- | benzenesulfonyl | |
| benzazepine (E25) | chloride | |
| 3-Cyclopentyl-7-(4-{[3-(methyloxy)phenyl]sulfonyl}-1- | 3-(methyloxy) | 470 |
| piperazinyl)-2,3,4,5-tetrahydro-1H-3-benzazepine | benzenesulfonyl | |
| (E26) | chloride | |
| 3-Cyclopentyl-7-(4-{[4-(methyloxy)Phenyl]sulfonyl}-1- | 4-(methyloxy) | 470 |
| piperazinyl)-2,3,4,5-tetrahydro-1H-3-benzazepine | benzenesulfonyl | |
| (E27) | chloride | |
| 2,6-Dichloro-4-{[4-(3-cyclopentyl-2,3,4,5-tetrahydro- | 3,5-dichloro-4- | 525 |
| 1H-3-benzazepin-7-yl)-1-piperazinyl]sulfonyl}phenol | hydroxy | |
| (E28) | benzenesulfonyl | |
| | chloride | |
| 3-Cyclopentyl-7-[4-(8-quinolinylsulfonyl)-1-piperazinyl]- | 8-quinoline sulfonyl | 491 |
| 2,3,4,5-tetrahydro-1H-3-benzazepine (E29) | chloride | |
| 3-Cyclopentyl-7-[4-(5-isoquinolinylsulfonyl)-1- | 5-isoquinoline | 491 |
| piperazinyl]-2,3,4,5-tetrahydro-1H-3-benzazepine | sulfonyl chloride | |
| (E30) | | |
| 3-Cyclopentyl-7-{4-[(1-methyl-1H-imidazol-4- | 1-methyl-1H- | 444 |
| yl)sulfonyl]-1-piperazinyl}-2,3,4,5-tetrahydro-1H-3- | imidazole-4- | |
| benzazepine (E31) | sulfonyl chloride | |
| 3-Cyclopentyl-7-{4-[2,4-dimethyl-1,3-thiazol-5- | 2,4-dimethyl-1,3- | 475 |
| yl)sulfonyl]-1-piperazinyl}-2,3,4,5-tetrahydro-1H-3- | thiazole-5-sulfonyl | |
| benzazepine (E32) | chloride | |
| 3-Cyclopentyl-7-{4-[(1,3,5-trimethyl-1H-pyrazol-4- | 1,3,5-trimethyl-1H- | 472 |
| yl)sulfonyl]-1-piperazinyl}-2,3,4,5-tetrahydro-1H-3- | pyrazole-4-sulfonyl | |
| benzazepine (E33) | chloride | |
| 3-Cyclopentyl-7-[4-(3-thienylsulfonyl)-1-piperazinyl]- | 3-thiophenesulfonyl | 446 |
| 2,3,4,5-tetrahydro-1H-3-benzazepine (E34) | chloride | |
| 7-[4-(1-Benzothien-3-ylsulfonyl)-1-piperazinyl]-3- | 1-benzothiophene- | 496 |
| cyclopentyl-2,3,4,5-tetrahydro-1H-3-benzazepine | 3-sulfonyl chloride | |
| (E35) | | |
| 4-(3-Cyclopentyl-2,3,4,5-tetrahydro-1H-3-benzazepin- | dimethylsulfamoyl | 407 |
| 7-yl)-N,N-dimethyl-1-piperazinesulfonamide (E36) | chloride | |
| 3-Cyclopentyl-7-[4-(thieno[2,3-b]pyridin-2-ylsulfonyl)-1- | thieno[2,3- | 497 |
| piperazinyl]-2,3,4,5-tetrahydro-1H-3-benzazepine | b]pyridine-2- | |
| (E37) | sulfonyl chloride | |
| 3-Cyclopentyl-7-{4-[(2,2,2-trifluoroethyl)sulfonyl]-1- | 2,2,2- | 446 |
| piperazinyl}-2,3,4,5-tetrahydro-1H-3-benzazepine | trifluoroethane | |
| (E38) | sulfonyl chloride | |
| 3-Cyclopentyl-7-{4-[(phenylmethyl)sulfonyl]-1- | Phenylmethane | 454 |
| piperazinyl}-2,3,4,5-tetrahydro-1H-3-benzazepine | sulfonyl chloride | |
| (E39) | | |
| 3-Cyclopentyl-7-{4-[(1-methylethyl)sulfonyl]-1- | 2-propanesulfonyl | 406 |
| piperazinyl}-2,3,4,5-tetrahydro-1H-3-benzazepine | chloride | |
| (E40) | | |
| 3-Cyclopentyl-7-{4-[(4-methylphenyl)sulfonyl]-1- | 4-methylbenzene | 454 |
| piperazinyl}-2,3,4,5-tetrahydro-1H-3-benzazepine | sulfonyl chloride | |
| (E41) | | |
| 7-{4-[(4-Chlorophenyl)sulfonyl]-1-piperazinyl}-3- | 4-chlorobenzene | 475 |
| cyclopentyl-2,3,4,5-tetrahydro-1H-3-benzazepine | sulfonyl chloride | |
| (E42) | | |
| 7-{4-[(2-Chlorophenyl)sulfonyl]-1-piperazinyl}-3- | 2-chlorobenzene | 475 |
| cyclopentyl-2,3,4,5-tetrahydro-1H-3-benzazepine | sulfonyl chloride | |
| (E43) | | |
| 7-{4-[(3-Chlorophenyl)sulfonyl]-1-piperazinyl}-3- | 3-chlorobenzene | 475 |
| cyclopentyl-2,3,4,5-tetrahydro-1H-3-benzazepine | sulfonyl chloride | |
| (E44) | | |
| 3-Cyclopentyl-7-{4-[(2,3-dichlorophenyl)sulfonyl]-1- | 2,3- | 509 |
| piperazinyl}-2,3,4,5-tetrahydro-1H-3-benzazepine | dichlorobenzene | |
| (E45) | sulfonyl chloride | |
| 3-Cyclopentyl-7-(4-{[4-(1,1- | 4-(1,1- | 496 |
| dimethylethyl)phenyl]sulfonyl}-1-piperazinyl)-2,3,4,5- | dimethylethyl) | |
| tetrahydro-1H-3-benzazepine (E46) | benzenesulfonyl | |
| | chloride | |
| N-(4-{[4-(3-Cyclopentyl-2,3,4,5-tetrahydro-1H-3- | 4-(acetylamino) | 497 |
| benzazepin-7-yl)-1- | benzenesulfonyl | |
| piperazinyl]sulfonyl}phenyl)acetamide (E47) | chloride | |
| 1-(4-{[4-(3-Cyclopentyl-2,3,4,5-tetrahydro-1H-3- | 4-acetylbenzene | 482 |
| benzazepin-7-yl)-1- | sulfonyl chloride | |
| piperazinyl]sulfonyl}phenyl)ethanone (E48) | | |
| 3-Cyclopentyl-7-(4-{[2-(1-naphthalenyl)ethyl]sulfonyl}- | 2-(1-naphthalenyl) | 518 |
| 1-piperazinyl)-2,3,4,5-tetrahydro-1H-3-benzazepine | ethanesulfonyl | |
| (E49) | chloride | |
| 4-{[4-(3-Cyclopentyl-2,3,4,5-tetrahydro-1H-3- | 4-(chlorosulfonyl) | 484 |
| benzazepin-7-yl)-1-piperazinyl]sulfonyl}benzoic acid | benzoic acid | |
| (E50) | | |
| 3-Cyclopentyl-7-(4-{[4-(trifloromethyl)phenyl]sulfonyl}- | 4-(trifluoromethyl) | 508 |
| 1-piperazinyl)-2,3,4,5-tetrahydro-1H-3-benzazepine | benzenesulfonyl | |
| (E51) | chloride | |
| 7-[4-(4-Biphenylylsulfonyl)-1-piperazinyl]-3-cyclopentyl- | 4-biphenylsulfonyl | 516 |
| 2,3,4,5-tetrahdro-1H-3-benzazepine (E52) | chloride | |
| 3-Cyclopentyl-7-(4-{[5-(1,3-oxazol-5-yl)-2- | 5-(1,3-oxazol-5-yl)- | 513 |
| thienyl]sulfonyl}-1-piperazinyl)-2,3,4,5-tetrahydro-1H-3- | 2-thiophenesulfonyl | |
| benzazepine (E53) | chloride | |
| 3-Cyclopentyl-7-[4-(2-naphthalenylsulfonyl)-1- | 2-naphthalene | 490 |
| pierazinyl]-2,3,4,5-tetrahydro-1H-3-benzazepine | sulfonyl chloride | |
| (E54) | | |
| 5-{[4-(3-Cyclopentyl-2,3,4,5-tetrahydro-1H-3- | 5-(dimethylamino)- | 533 |
| benzazepin-7-yl)-1-piperazinyl]sulfonyl}-N,N-dimethyl- | 1-naphthalene | |
| 1-naphthalenamine (E55) | sulfonyl chloride | |
| 3-Cyclopentyl-7-(4-{[(E)-2-phenylethenyl]sulfonyl}-1- | (E)-2-phenylethene | 466 |
| piperazinyl)-2,3,4,5-tetrahydro-1H-3-benzazepine | sulfonyl chloride | |
| (E56) | | |
| 3-Cyclopentyl-7-(4-{[4-(1-methylethyl)phenyl]sulfonyl}- | 4-(1-methylethyl) | 482 |
| 1-piperazinyl)-2,3,4,5-tetrahydro-1H-3-benzazepine | benzenesulfonyl | |
| (E57) | chloride | |
| 7-{4-[(3-Chloro-2-methylphenyl)sulfonyl]-1-piperazinyl}- | 3-chloro-2- | 489 |
| 3-cyclopentyl-2,3,4,5-tetrahydro-1H-3-benzazepine | methylbenzene | |
| (E58) | sulfonyl chloride | |
| 3-Cyclopentyl-7-[4-(1-naphthalenylsulfonyl)-1- | 1-naphthalene | 490 |
| piperazinyl]-2,3,4,5-tetrahydro-1H-3-benzazepine | sulfonyl chloride | |
| (E59) | | |
| 7-{4-[(5-Chloro-2-thienyl)sulfonyl]-1-piperazinyl}-3- | 5-chloro-2- | 481 |
| cyclopentyl-2,3,4,5-tetrahydro-1H-3-benzazepine | thiophenesulfonyl | |
| (E60) | chloride | |
| 3-Cyclopentyl-7-[4-(methylsulfonyl)-1-piperazinyl]- | methanesulfonyl | 378 |
| 2,3,4,5-tetrahydro-1H-3-benzazepine (E61) | chloride | |
| 3-Cyclopentyl-7-(4-{[3-(trifluoromethyl)Phenyl]sulfonyl} | 3-(trifluoromethyl) | 508 |
| 1-piperazinyl)-2,3,4,5-tetrahydro-1H-3-benzazepine | benzenesulfonyl | |
| (E62) | chloride | |
| 3-Cyclopentyl-7-(4-{[5-(2-pyridinyl)-2-thienyl]sulfonyl}- | 5-(2-pyridinyl)-2- | 523 |
| 1-piperazinyl)-2,3,4,5-tetrahydro-1H-3-benzazepine | thiophenesulfonyl | |
| (E63) | chloride | |
| 7-{4-[(4-Chloro-1-benzothien-2-yl)sulfonyl]-1- | 4-chloro-1- | 531 |
| piperazinyl}-3-cyclopentyl-2,3,4,5-tetrahydro-1H-3- | benzothiophene-2- | |
| benzazepine (E64) | sulfonyl chloride | |
| 7-[4-(2,1,3-Benzoxadiazol-4-ylsulfonyl)-1-piperazinyl]- | 2,1,3- | 482 |
| 3-cyclopentyl-2,3,4,5-tetrahydro-1H-3-benzazepine | benzoxadiazole-4- | |
| (E65) | sulfonyl chloride | |
| 3-Cyclopentyl-7-{4-[(1,2-dimethyl-1H-imidazol-4- | 1,2-dimethyl-1H- | 458 |
| yl)sulfonyl]-1-piperazinyl}-2,3,4,5-tetrahydro-1H-3- | imidazole-4- | |
| benzazepine (E66) | sulfonyl chloride | |
| N-(5-{[4-(3-Cyclopentyl-2,3,4,5-tetrahydro-1H-3- | 2-(acetylamino)-4- | 518 |
| benzazepin-7-yl)-1-piperazinyl]sulfonyl}-4-methyl-1,3- | methyl-1,3-thiazole- | |
| thiazol-2-yl)acetamide (E67) | 5-sulfonyl chloride | |
| 3-Cyclopentyl-7-{4-[(3,5-dichlorophenyl)sulfonyl]-1- | 3,5- | 509 |
| piperazinyl}-2,3,4,5-tetrahydro-1H-3-benzazepine | dichlorobenzene | |
| (E68) | sulfonyl chloride | |
| 3-Cyclopentyl-7-[4-({4- | 4-[(trifluoromethyl) | 524 |
| [(trifluoromethyl)oxy]phenyl}sulfonyl)-1-piperazinyl]- | oxy]benzene | |
| 2,3,4,5-tetrahdro-1H-3-benzazepine (E69) | sulfonyl chloride | |
| 3-Cyclopentyl-7-(4-{[2-(trifluoromethyl)phenyl]sulfonyl}- | 2-(trifluoromethyl) | 508 |
| 1-piperazinyl)-2,3,4,5-tetrahydro-1H-3-benzazepine | benzenesulfonyl | |
| (E70) | chloride | |
| 3-Cyclopentyl-7-{4-[(3,5-dimethyl-4- | 3,5-dimethyl-4- | 459 |
| isoxazolyl)sulfonyl]-1-piperazinyl}-2,3,4,5-tetrahydro- | isoxazolesulfonyl | |
| 1H-3-benzazepine (E71) | chloride | |
| 3-Cyclopentyl-7-(4-{[6-(phenyloxy)-3- | 6-(phenyloxy)-3- | 533 |
| pyridinyl]sulfonyl}-1-piperazinyl)-2,3,4,5-tetrahydro-1H- | pyridinesulfonyl | |
| 3-benzazepine (E72) | chloride | |
| 3-Cyclopentyl-7-[4-(phenylsulfonyl)-1-piperazinyl]- | benzenesulfonyl | 440 |
| 2,3,4,5-tetrahydro-1H-3-benzazepine (E73) | chloride | |
| 3-Cyclopentyl-7-{4-[(5-methyl-1-phenyl-1H-pyrazol-4- | 5-methyl-1-phenyl- | 520 |
| yl)sulfonyl]-1-piperazinyl}-2,3,4,5-tetrahydro-1H-3- | 1H-pyrazole-4- | |
| benzazepine (E74) | sulfonyl chloride | |
| 7-(4-{[(4-Chlorophenyl)methyl]sulfonyl}-1-piperazinyl)- | (4-chlorophenyl) | 489 |
| 3-cyclopentyl-2,3,4,5-tetrahydro-1H-3-benzazepine | methanesulfonyl | |
| (E75) | chloride | |
| 3-Cyclopentyl-7-[4-({[4- | [4-(trifluoromethyl) | 522 |
| (trifluoromethyl)phenyl]methyl}sulfonyl)-1-piperazinyl]- | phenyl]methane | |
| 2,3,4,5-tetrahydro-1H-3-benzazepine (E76) | sulfonyl chloride | |
| 3-Cyclopentyl-7-[4-(2,3-dihydro-1-benzofuran-5- | 2,3-dihydro-1- | 482 |
| ylsulfonyl)-1-piperazinyl]-2,3,4,5-tetrahydro-1H-3- | benzofuran-5- | |
| benzazepine (E77) | sulfonyl chloride | |
| 6-{[4-(3-Cyclopentyl-2,3,4,5-tetrahydro-1H-3- | 2-oxo-2H- | 508 |
| benzazepin-7-yl)-1-piperazinyl]sulfonyl}-2H-chromen- | chromene-6- | |
| 2-one (E78) | sulfonyl chloride | |
| 3-Cyclopentyl-7-(4-{[5-(3-isoxazolyl)-2-thienyl]sulfonyl}- | 5-(3-isoxazolyl)-2- | 513 |
| 1-piperazinyl)-2,3,4,5-tetrahydro-1H-3-benzazepine | thiophenesulfonyl | |
| (E79) | chloride | |
| 7-[4-(2,1,3-Benzothiadiazol-5-ylsulfonyl)-1-piperazinyl]- | 2,1,3- | 498 |
| 3-cyclopentyl-2,3,4,5-tetrahydro-1H-3-benzazepine | benzothiadiazole-5- | |
| (E80) | sulfonyl chloride | |
| 7-(4-{[5-Chloro-2-(methyloxy)phenyl]sulfonyl}-1- | 5-chloro-2- | 505 |
| piperazinyl)-3-cyclopentyl-2,3,4,5-tetrahydro-1H-3- | (methyloxy) | |
| benzazepine (E81) | benzenesulfonyl | |
| | chloride | |
| 3-Cyclopentyl-7-{4-[(5-fluoro-2-(methylphenyl)sulfonyl]- | 5-fluoro-2- | 472 |
| 1-piperazinyl}-2,3,4,5-tetrahydro-1H-3-benzazepine | methylbenzene | |
| (E82) | sulfonyl chloride | |
| 7-{4-[(4-Bromo-2-ethylphenyl)sulfonyl]-1-piperazinyl}- | 4-bromo-2- | 547 |
| 3-cyclopentyl-2,3,4,5-tetrahydro-1H-3-benzazepine | ethylbenzene | |
| (E83) | sulfonyl chloride | |
| 7-{4-[(6-Chloroimidazo[2,1-b][1,3]thiazol-5-yl)sulfonyl]- | 6- | 521 |
| 1-piperazinyl}-3-cyclopentyl-2,3,4,5-tetrahydro-1H-3- | chloroimidazo[2,1- | |
| benzazepine (E84) | b][1,3]thiazole-5- | |
| | sulfonyl chloride | |
| 3-Cyclopentyl-7-(4-{[3-(5-methyl-1,3,4-oxadiazol-2- | 3-(5-methyl-1,3,4- | 522 |
| yl)phenyl]sulfonyl}-1-piperazinyl)-2,3,4,5-tetrahydro- | oxadiazol-2- | |
| 1H-3-benzazepine (E85) | yl)benzenesulfonyl | |
| | chloride | |
| 3-Cyclopentyl-7-{4-[(2,5-dimethyl-3-thienyl)sulfonyl]-1- | 2,5-dimethyl-3- | 474 |
| piperazinyl}-2,3,4,5-tetrahydro-1H-3-benzazepine | thiophenesulfonyl | |
| (E86) | chloride | |
| 3-Cyclopentyl-7-(4-{[4-methyl-2- | 4-methyl-2- | 484 |
| (methyloxy)phenyl]sulfonyl}-1-piperazinyl)-2,3,4,5- | (methyloxy) | |
| tetrahydro-1H-3-benzazepine (E87) | benzenesulfonyl | |
| | chloride | |
| 3-Cyclopentyl-7-(4-{[2-(3-methylphenyl)ethyl]sulfonyl}- | 2-(3-methylphenyl) | 482 |
| 1-piperazinyl)-2,3,4,5-tetrahydro-1H-3-benzazepine | ethanesulfonyl | |
| (E88) | chloride | |
| 3-Cyclopentyl-7-(4-{[4-(methylsulfonyl)phenyl]sulfonyl}- | 4-(methylsulfonyl) | 518 |
| 1-piperazinyl)-2,3,4,5-tetrahydro-1H-3-benzazepine | benzenesulfonyl | |
| (E89) | chloride | |
| 3-Cyclopentyl-7-{4-[(3,4,5-trimethylphenyl)sulfonyl]-1- | 3,4,5- | 482 |
| piperazinyl}-2,3,4,5-tetrahydro-1H-3-benzazepine | trimethylbenzene | |
| (E90) | sulfonyl chloride | |

### EXAMPLE 91

### 4-{[4-(3-Cyclopentyl-2,3,4,5-tetrahydro-1H-3-benzazepin-7-yl)-1-piperazinyl]carbonyl}benzonitrile (E91)

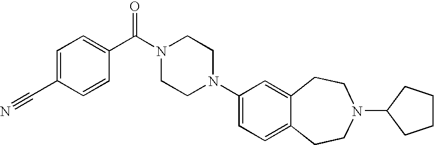

To a solution of 4-cyanobenzoic acid (18 mg, 0.07 mmol) in dichloromethane (2 ml) O-Benzotriazol-1-yl-N,N,N′,N′-tetramethyluronium hexafluorophosphate (22.7 mg, 0.06 mmol) was added. The reaction was stirred for 40 minutes before the addition of 3-cyclopentyl-7-(1-piperazinyl)-2,3,4,5-tetrahydro-1H-3-benzazepine (D6) (14.9 mg, 0.05 mmol) followed by morpholinomethyl-polystyrene resin (4.3 mmol/g, 34.8 mg, 0.15 mmol). The reaction mixture was shaken at room temperature for 12 hours. MP-carbonate resin (2.8 mmol/g, 0.18 g, 0.5 mmol) was added and the reaction was shaken for 1 day. The resin was filtered and washed 3 times with dichloromethane and the filtrate solutions were drained onto a SCX ion-exchange cartridge (Varian bond-elute, 500 mg). The cartridge was washed with methanol then 2M ammonia in methanol solution. Solvents were removed in vacuo and the crude residue was purified by column chromatography eluting with dichloromethane then ethyl acetate, then methanol to afford the title product (E91); MS (ES+) m/e 429 [M+H]⁺; ¹H NMR (CDCl₃) 7.73 (2H, d), 7.54 (2H, d), 7.01 (1H, d), 6.70-6.65 (2H, m), 3.92 (2H, brs), 3.62-3.47 (2H, m), 3.22-3.08 (4H, m), 2.91 (5H, m), 2.72 (4H, m), 1.87 (2H, m), 1.67 (2H, m), 1.56-1.47 (4H, m).

### EXAMPLES 92-190 (E92-190)

Examples 92-190 (E92-E190) were prepared using an analogous method to that described for Example 91 (E91) from 3-cyclopentyl-7-(1-piperazinyl)-2,3,4,5-tetrahydro-1H-3-benzazepine (D6) and the appropriate carboxylic acid as indicated in the table. No further purification was required in Examples 161-190 (E161-E190) after recovery of the title compound from the SCX ion-exchange cartridge. Secondary purification was performed by column chromatography eluting with dichloromethane then ethyl acetate, then methanol for Examples 92-93 (E92-E93), or by Mass Spectrometer-coupled High Performance Liquid Chromatography (SUPELCOSIL™ ABZ+PLUS 12 μM column, eluents: acetonitrile:water+0.1% v/v trifluoroacetic acid) for Examples 94-160 (E95-E160).

| | | LC/MS |
|---|---|---|
| Example | Acid | (M + H)⁺ |
| 3-Cyclopentyl-7-[4-(4-pyridinylcarbonyl)-1-piperazinyl]- | 4-pyridinecarboxylic | 405 |
| 2,3,4,5-tetrahydro-1H-3-benzazepine (E92) | acid | |
| 3-Cyclopentyl-7-[4-(cyclopentylcarbonyl)-1- | Cyclopentane | 396 |
| piperazinyl]-2,3,4,5-tetrahydro-1H-3-benzazepine | carboxylic acid | |
| (E93) | | |
| 3-Cyclopentyl-7-[4-(1H-indol-3-ylcarbonyl)-1- | 1H-indole-3- | 557 |
| piperazinyl]-2,3,4,5-tetrahydro-1H-3-benzazepine | carboxylic acid | |
| trifluoroacetate salt (E94) | | |
| 3-Cyclopertyl-7-(4-{[2-(phenyloxy)phenyl]carbonyl}-1- | 2- | 610 |
| piperazinyl)-2,3,4,5-tetrahydro-1H-3-benzazepine | (phenyloxy)benzoic | |
| trifluoroacetate salt (E95) | acid | |
| 3-Cyclopentyl-7-(4-{[2-(methyloxy)Phenyl]carbonyl}-1- | 2- | 548 |
| Piperazinyl)-2,3,4,5-tetrahydro-1H-3-benzazepine | (methyloxy)benzoic | |
| trifluoroacetate salt (E96) | acid | |
| 3-Cyclopentyl-7-{4-[(3,4-dichlorophenyl)carbonyl]-1- | 3,4-dichiorobenzoic | 587 |
| piperazinyl}-2,3,4,5-tetrahydro-1H-3-benzazepine | acid | |
| trifluoroacetate salt (E97) | | |
| 3-Cyclopentyl-7-[4-(2-phenylpropanoyl)-1-piperazinyl]- | 2-phenylpropanoic | 546 |
| 2,3,4,5-tetrahydro-1H-3-benzazepine trifluoroacetate | acid | |
| salt (E98) | | |
| 3-Cyclopentyl-7-(4-{[4-(1H-pyrazol-1-yl)phenyl]acetyl}- | [4-(1H-pyrazol-1- | 598 |
| 1-piperazinyl)-2,3,4,5-tetrahydro-1H-3-benzazepine | yl)phenyl]acetic acid | |
| trifluoroacetate salt (E99) | | |
| 3-Cyclopentyl-7-{4-[(4-methyl-2-phenyl-3- | 4-methyl-2-phenyl-3- | 598 |
| furanyl)carbonyl]-1-piperazinyl}-2,3,4,5-tetrahydro-1H- | furancarboxylic acid | |
| 3-benzazepine trifluoroacetate salt (E100) | | |
| 3-Cyclopentyl-7-(4-{[4-(1,1- | 4-(1,1-dimethylethyl) | 574 |
| dimethylethyl)phenyl]carbonyl}-1-piperazinyl)-2,3,4,5- | benzoic acid | |
| tetrahydro-1H-3-benzazepine trifluoroacetate salt | | |
| (E101) | | |
| (3-{[4-(3-Cyclopentyl-2,3,4,5-tetrahydro-1H-3- | 3-(phenylcarbonyl) | 622 |
| benzazepin-7-yl)-1- | benzoic acid | |
| piperazinyl]carbonyl}phenyl)(phenyl)methanone | | |
| trifluoroacetate salt (E102) | | |
| 3-Cyclopentyl-7-[4-(2,3-dihydro-1-benzofuran-2- | 2,3-dihydro-1- | 560 |
| ylcarbonyl)-1-piperazinyl]-2,3,4,5-tetrahydro-1H-3- | benzofuran-2- | |
| benzazepine trifluoroacetate salt (E103) | carboxylic acid | |
| 7-[4-(4-Biphenylylcarbonyl)-1-piperazinyl]-3- | 4-biphenylcarboxylic | 594 |
| cyclopentyl-2,3,4,5-tetrahydro-1H-3-benzazepine | acid | |
| trifluoroacetate salt (E104) | | |
| 7-{4-[(5-Chloro-1-benzothien-2-yl)carbonyl]-1- | 5-chloro-1- | 609 |
| piperazinyl}-3-cyclopentyl-2,3,4,5-tetrahydro-1H-3- | benzothiophene-2- | |
| benzazepine trifluoroacetate salt (E105) | carboxylic acid | |
| 7-[4-(1-Benzothien-2-ylcarbonyl)-1-piperazinyl]-3- | 1-benzothiophene-2- | 574 |
| cyclopentyl-2,3,4,5-tetrahydro-1H-3-benzazepine | carboxylic acid | |
| trifluoroacetate salt (E106) | | |
| 3-Cyclopentyl-7-{4-[(5-methyl-4-phenyl-2- | 5-methyl-4-phenyl-2- | 614 |
| thienyl)carbonyl]-1-piperazinyl}-2,3,4,5-tetrahydro-1H- | thiophenecarboxylic | |
| 3-benzazepine trifluoroacetate salt (E107) | acid | |
| 7-[4-(1,3-Benzothiazol-6-ylcarbonyl)-1-piperazinyl]-3- | 1,3-benzothiazole-6- | 575 |
| cyclopentyl-2,3,4,5-tetrahydro-1H-3-benzazepine | carboxylic acid | |
| trifluoroacetate salt (E108) | | |
| 3-Cyclopentyl-7-[4-(phenylcarbonyl)-1-piperazinyl]- | benzoic acid | 518 |
| 2,3,4,5-tetrahydro-1H-3-benzazepine trifluoroacetate | | |
| salt (E109) | | |
| 3-Cyclopentyl-7-[4-(2-naphthalenylcarbonyl)-1- | 2-naphthalene | 568 |
| piperazinyl]-2,3,4,5-tetrahydro-1H-3-benzazepine | carboxylic acid | |
| trifluoroacetate salt (E110) | | |
| 1-(4-{[4-(3-Cyclopentyl-2,3,4,5-tetrahydro-1H-3- | 4-acetylbenzoic acid | 560 |
| benzazepin-7-yl)-1-piperazinyl]carbonyl}phenyl) | | |
| ethanone trifluoroacetate salt (E111) | | |
| 3-Cyclopentyl-7-(4-{[4-(1-methylethyl)phenyl]carbonyl}- | 4-(1- | 560 |
| 1-piperazinyl)-2,3,4,5-tetrahydro-1H-3-benzazepine | methylethyl)benzoic | |
| trifluoroacetate salt (E112) | acid | |
| 3-Cyclopentyl-7-(4-{[4- | 4-(trifluoromethyl) | 586 |
| (trifluoromethyl)phenyl]carbonyl}-1-piperazinyl)-2,3,4,5- | benzoic acid | |
| tetrahydro-1H-3-benzazepine trifluoroacetate salt | | |
| (E113) | | |
| 3-Cyclopentyl-7-[4-({3- | 3- | 602 |
| [(trifluoromethyl)oxy]phenyl}carbonyl)-1-piperazinyl]- | [(trifluoromethyl)oxy] | |
| 2,3,4,5-tetrahydro-1H-3-benzazepine trifluoroacetate | benzoic acid | |
| salt (E114) | | |
| 7-(4-{[2-Bromo-5-(methyloxy)phenyl]carbonyl}-1- | 2-bromo-5- | 627 |
| piperazinyl)-3-cyclopentyl-2,3,4,5-tetrahydro-1H-3- | (methyloxy)benzoic | |
| benzazepine trifluoroacetate salt (E115) | acid | |
| N-{2-[4-(3-Cyclopentyl-2,3,4,5-tetrahydro-1H-3- | N-[(2-methylphenyl) | 589 |
| benzazepin-7-yl)-1-piperazinyl]-2-oxoethyl}-2- | carbonyl]glycine | |
| methylbenzamide trifluoroacetate salt (E116) | | |
| 3-Cyclopentyl-7-[4-(1,3-dihydro-2H-isoindol-2-ylacetyl)- | 1,3-dihydro-2H- | 573 |
| 1-piperazinyl]-2,3,4,5-tetrahydro-1H-3-benzazepine | isoindol-2-ylacetic | |
| trifluoroacetate salt (E117) | acid | |
| 7-{4-[(3-Chlorophenyl)carbonyl]-1-piperazinyl}-3- | 3-chlorobenzoic acid | 553 |
| cyclopentyl-2,3,4,5-tetrahydro-1H-3-benzazepine | | |
| trifluoroacetate salt (E118) | | |
| 7-{4-[(4-Chlorophenyl)carbonyl]-1-piperazinyl}-3- | 4-chlorobenzoic acid | 553 |
| cyclopentyl-2,3,4,5-tetrahydro-1H-3-benzazepine | | |
| trifluoroacetate salt (E119) | | |
| 7-{4-[(2-Chlorophenyl)carbonyl]-1-piperazinyl}-3- | 2-chlorobenzoic acid | 553 |
| cyclopentyl-2,3,4,5-tetrahydro-1H-3-benzazepine | | |
| trifluoroacetate salt (E120) | | |
| 3-Cyclopentyl-7-{4-[(4-nitrophenyl)carbonyl]-1- | 4-nitrobenzoic acid | 563 |
| piperazinyl}-2,3,4,5-tetrahydro-1H-3-benzazepine | | |
| trifluoroacetate salt (E121) | | |
| N-(4-{[4-(3-Cyclopentyl-2,3,4,5-tetrahydro-1H-3- | 4-(acetylamino) | 575 |
| benzazepin-7-yl)-1- | benzoic acid | |
| piperazinyl]carbonyl}phenyl)acetamide trifluoroacetate | | |
| salt (E122) | | |
| (4-{[4-(3-Cyclopentyl-2,3,4,5-tetrahydro-1H-3- | 4-(dimethylamino) | 561 |
| benzazepin-7-yl)-1- | benzoic acid | |
| piperazinyl]carbonyl}phenyl)dimethylamine | | |
| trifluoroacetate salt (E123) | | |
| 3-Cyclopentyl-7-(4-{[3-(methyloxy)phenyl]carbonyl}-1- | 3-(methyloxy) | 548 |
| piperazinyl)-2,3,4,5-tetrahydro-1H-3-benzazepine | benzoic acid | |
| trifluoroacetate salt (E124) | | |
| 3-Cyclopentyl-7-[4-({4-[(1- | 4-[(1- | 576 |
| methylethyl)oxy]phenyl}carbonyl)-1-piperazinyl]- | methylethyl)oxy] | |
| 2,3,4,5-tetrahydro-1H-3-benzazepine trifluoroacetate | benzoic acid | |
| salt (E125) | | |
| 3-Cyclopentyl-7-(4-{[4-(methyloxy)phenyl]carbonyl}-1- | 4- | 548 |
| piperazinyl)-2,3,4,5-tetrahydro-1H-3-benzazepine | (methyloxy)benzoic | |
| trifluoroacetate salt (E126) | acid | |
| 7-(4-{[3-Chloro-5-(methyloxy)phenyl]carbonyl}-1- | 3-chloro-5- | 583 |
| piperazinyl)-3-cyclopentyl-2,3,4,5-tetrahydro-1H-3- | (methyloxy)benzoic | |
| benzazepine trifluoroacetate salt (E127) | acid | |
| 7-[4-(1,3-Benzodioxol-5-ylacetyl)-1-piperazinyl]-3- | 1,3-benzodioxol-5- | 576 |
| cyclopentyl-2,3,4,5-tetrahydro-1H-3-benzazepine | ylacetic acid | |
| trifluoroacetate salt (E128) | | |
| 3-Cyclopentyl-7-(4-{[5-(phenylmethyl)-2- | 5-(phenylmethyl)-2- | 598 |
| furanyl]carbonyl}-1-piperazinyl)-2,3,4,5-tetrahydro-1H- | furancarboxylic acid | |
| 3-benzazepine trifluoroacetate salt (E129) | | |
| 3-Cyclopentyl-7-[4-(3-furanylcarbonyl)-1-piperazinyl]- | 3-furancarboxylic | 508 |
| 2,3,4,5-tetrahydro-1H-3-benzazepine trifluoroacetate | acid | |
| salt (E130) | | |
| 3-Cyclopentyl-7-(4-{[3-(2-furanyl)phenyl]carbonyl}-1- | 3-(2-furanyl)benzoic | 584 |
| piperazinyl)-2,3,4,5-tetrahydro-1H-3-benzazepine | acid | |
| trifluoroacetate salt (E131) | | |
| 3-Cyclopentyl-7-[4-(cyclopropylcarbonyl)-1- | Cyclopropane | 482 |
| piperazinyl]-2,3,4,5-tetrahydro-1H-3-benzazepine | carboxylic acid | |
| trifluoroacetate salt (E132) | | |
| 3-Cyclopentyl-7-[4-(3,3-dimethylbutanoyl)-1- | 3,3-dimethylbutanoic | 512 |
| piperazinyl]-2,3,4,5-tetrahydro-1H-3-benzazepine | acid | |
| trifluoroacetate salt (E133) | | |
| 3-Cyclopentyl-7-{4-[(2E)-3-phenyl-2-propenoyl]-1- | (2E)-3-phenyl-2- | 544 |
| piperazinyl}-2,3,4,5-tetrahydro-1H-3-benzazepine | propenoic acid | |
| trifluoroacetate salt (E134) | | |
| 3-Cyclopentyl-7-[4-({cis-4-[(1,1- | cis-4-[(1,1- | 596 |
| dimethylethyl)oxy]cyclohexyl}carbonyl)-1-piperazinyl]- | dimethylethyl)oxy] | |
| 2,3,4,5-tetrahydro-1H-3-benzazepine trifluoroacetate | cyclohexane | |
| salt (E135) | carboxylic acid | |
| 3-Cyclopentyl-7-(4-{[1-(1-methylethyl)-4- | 1-(1-methylethyl)-4- | 567 |
| piperidinyl]carbonyl}-1-piperazinyl)-2,3,4,5-tetrahydro- | piperidinecarboxylic | |
| 1H-3-benzazepine trifluoroacetate salt (E136) | acid | |
| 1-{2-[4-(3-Cyclopentyl-2,3,4,5-tetrahydro-1H-3- | (2-oxo-1- | 553 |
| benzazepin-7-yl)-1-piperazinyl]-2-oxoethyl}-2- | piperidinyl)acetic | |
| piperidinone trifluoroacetate salt (E137) | acid | |
| 3-Cyclopentyl-7-(4-{[(1-methylethyl)oxy]acetyl}-1- | [(1-methylethyl)oxy] | 514 |
| piperazinyl)-2,3,4,5-tetrahydro-1H-3-benzazepine | acetic acid | |
| trifluoroacetate salt (E138) | | |
| N—{(1R)-2-[4-(3-Cyclopentyl-2,3,4,5-tetrahydro-1H-3- | (2R)-(acetylamino) | 589 |
| benzazepin-7-yl)-1-piperazinyl]-2-oxo-1- | (phenyl)ethanoic | |
| phenylethyl}acetamide trifluoroacetate salt (E139) | acid | |
| 3-Cyclopentyl-7-{4-[1-(phenylmethyl)-L-prolyl]-1- | 1-(phenylmethyl)-L- | 601 |
| piperazinyl}-2,3,4,5-tetrahydro-1H-3-benzazepine | proline | |
| trifluoroacetate salt (E140) | | |
| 3-Cyclopentyl-7-[4-(2,3-dihydro-1H-inden-2- | 2,3-dihydro-1H- | 558 |
| ylcarbonyl)-1-piperazinyl]-2,3,4,5-tetrahydro-1H-3- | indene-2-carboxylic | |
| benzazepine trifluoroacetate salt (E141) | acid | |
| 3-Cyclopentyl-7-{4-[3-methyl-2- | 3-methyl-2- | 588 |
| (phenylmethyl)butanoyl]-1-piperazinyl}-2,3,4,5- | (phenylmethyl) | |
| tetrahydro-1H-3-benzazepine trifluoroacetate salt | butanoic acid | |
| (E142) | | |
| 3-Cyclopentyl-7-{4-[(1,1-dioxido-3,4-dihydro-2H-1- | 3,4-dihydro-2H-1- | 622 |
| benzothiopyran-6-yl)carbonyl]-1-piperazinyl}-2,3,4,5- | benzothiopyran-6- | |
| tetrahydro-1H-3-benzazepine trifluoroacetate salt | carboxylic acid 1,1- | |
| (E143) | dioxide | |
| 3-Cyclopentyl-7-(4-{[4- | 4-(methylsulfonyl) | 596 |
| (methylsulfonyl)phenyl]carbonyl}-1-piperazinyl)- | benzoic acid | |
| 2,3,4,5-tetrahydro-1H-3-benzazepine trifluoroacetate | | |
| salt (E144) | | |
| 3-Cyclopentyl-7-[4-(3,4-dihydro-2H-chromen-2- | 3,4-dihydro-2H- | 574 |
| ylcarbonyl)-1-piperazinyl]-2,3,4,5-tetrahydro-1H-3- | chromene-2- | |
| benzazepine trifluoroacetate salt (E145) | carboxylic acid | |
| 3-Cyclopentyl-7-[4-(2,3-dihydro-1-benzofuran-7- | 2,3-dihydro-1- | 560 |
| ylcarbonyl)-1-piperazinyl]-2,3,4,5-tetrahydro-1H-3- | benzofuran-7- | |
| benzazepine trifluoroacetate salt (E146) | carboxylic acid | |
| 3-Cyclopentyl-7-(4-{[4-(3-pyridinyl)phenyl]carbonyl}-1- | 4-(3- | 595 |
| piperazinyl)-2,3,4,5-tetrahydro-1H-3-benzazepine | pyridinyl)benzoic | |
| trifluoroacetate salt (E147) | acid | |
| 3-Cyclopentyl-7-[4-(3-quinolinylcarbonyl)-1- | 3- | 569 |
| piperazinyl]-2,3,4,5-tetrahydro-1H-3-benzazepine | quinolinecarboxylic | |
| trifluoroacetate salt (E148) | acid | |
| 3-Cyclopentyl-7-[4-(pyrazolo[1,5-a]pyridin-3- | pyrazolo[1,5- | 558 |
| ylcarbonyl)-1-piperazinyl]-2,3,4,5-tetrahydro-1H-3- | a]pyridine-3- | |
| benzazepine trifluoroacetate salt (E149) | carboxylic acid | |
| 3-Cyclopentyl-7-[4-(5-isoquinolinylcarbonyl)-1- | 5-isoquinoline | 569 |
| piperazinyl]-2,3,4,5-tetrahydro-1H-3-benzazepine | carboxylic acid | |
| trifluoroacetate salt (E150) | | |
| 7-[4-(1-Benzothien-3-ylcarbonyl)-1-piperazinyl]-3- | 1-benzothiophene-3- | 574 |
| cyclopentyl-2,3,4,5-tetrahydro-1H-3-benzazepine | carboxylic acid | |
| trifluoroacetate salt (E151) | | |
| 3-Cyclopentyl-7-(4-{[5-(2-pyridinyl)-2-thienyl]carbonyl}- | 5-(2-pyridinyl)-2- | 601 |
| 1-piperazinyl)-2,3,4,5-tetrahydro-1H-3-benzazepine | thiophenecarboxylic | |
| trifluoroacetate salt (E152) | acid | |
| 3-Cyclopentyl-7-{4-[(1,3-dimethyl-1H-thieno[2,3- | 1,3-dimethyl-1H | 592 |
| c]pyrazol-5-yl)carbonyl]-1-piperazinyl}-2,3,4,5- | thieno[2,3- | |
| tetrahydro-1H-3-benzazepine trifluoroacetate salt | c]pyrazole-5- | |
| (E153) | carboxylic acid | |
| 3-Cyclopentyl-7-{4-[(4-methyl-2-phenyl-1,3-thiazol-5- | (4-methyl-2-phenyl- | 629 |
| yl)acetyl]-1-piperazinyl}-2,3,4,5-tetrahydro-1H-3- | 1,3-thiazol-5- | |
| benzazepine trifluoroacetate salt (E154) | yl)acetic acid | |
| 7-[4-(1,3-Benzothiazol-2-ylcarbonyl)-1-piperazinyl]-3- | 1,3-benzothiazole-2- | 575 |
| cyclopentyl-2,3,4,5-tetrahydro-1H-3-benzazepine | carboxylic acid | |
| trifluoroacetate salt (E155) | | |
| 3-Cyclopentyl-7-[4-(imidazo[2,1-b][1,3]thiazol-5- | imidazo[2,1- | 578 |
| ylacetyl)-1-piperazinyl]-2,3,4,5-tetrahydro-1H-3- | b][1,3]thiazol-5- | |
| benzazepine trifluoroacetate salt (E156) | ylacetic acid | |
| 3-Cyclopentyl-7-(4-{[4-(3,5-dimethyl-1H-pyrazol-1- | 4-(3,5-dimethyl-1H- | 612 |
| yl)phenyl]carbonyl}-1-piperazinyl)-2,3,4,5-tetrahydro- | pyrazol-1-yl)benzoic | |
| 1H-3-benzazepine trifluoroacetate salt (E157) | acid | |
| 3-Cyclopentyl-7-{4-[(2,4-dimethyl-1,3-oxazol-5- | 2,4-dimethyl-1,3- | 537 |
| yl)carbonyl]-1-piperazinyl}-2,3,4,5-tetrahydro-1H-3- | oxazole-5-carboxylic | |
| benzazepine trifluoroacetate salt (E158) | acid | |
| 3-Cyclopentyl-7-[4-(4-pyridinylacetyl)-1-piperazinyl]- | 4-pyridinylacetic | 533 |
| 2,3,4,5-tetrahydro-1H-3-benzazepine trifluoroacetate | acid | |
| salt (E159) | | |
| 6-{2-[4-(3-Cyclopentyl-2,3,4,5-tetrahydro-1H-3- | (2-oxo-1,2-dihydro- | 599 |
| benzazepin-7-yl)-1-piperazinyl]-2-oxoethyl}-2(1H)- | 6-quinolinyl)acetic | |
| quinolinone trifluoroacetate salt (E160) | acid | |
| 3-Cyclopentyl-7-[4-(phenylacetyl)-1-piperazinyl]- | phenylacetic acid | 418 |
| 2,3,4,5-tetrahydro-1H-3-benzazepine (E161) | | |
| 3-Cyclopentyl-7-(4-{[4-(1-methylethyl)phenyl]acetyl}-1- | [4-(1- | 460 |
| piperazinyl)-2,3,4,5-tetrahydro-1H-3-benzazepine | methylethyl)phenyl] | |
| (E162) | acetic acid | |
| 3-Cyclopentyl-7-[4-(2-naphthalenylacetyl)-1- | 2-naphthalenylacetic | 468 |
| piperazinyl]-2,3,4,5-tetrahydro-1H-3-benzazepine | acid | |
| (E163) | | |
| 3-Cyclopentyl-7-[4-(diphenylacetyl)-1-piperazinyl]- | diphenylacetic acid | 494 |
| 2,3,4,5-tetrahydro-1H-3-benzazepine (E164) | | |
| 3-Cyclopentyl-7-(4-{[4-(trifluoromethyl)Phenyl]acetyl}-1- | [4-(trifluoromethyl) | 486 |
| piperazinyl)-2,3,4,5-tetrahydro-1H-3-benzazepine | phenyl]acetic acid | |
| (E165) | | |
| 7-{4-[(4-Chlorophenyl)acetyl]-1-piperazinyl}-3- | (4-chlorophenyl) | 453 |
| cyclopentyl-2,3,4,5-tetrahydro-1H-3-benzazepine | acetic acid | |
| (E166) | | |
| 3-Cyclopentyl-7-(4-{[4-(methyloxy)phenyl]acetyl}-1- | [4- | 448 |
| piperazinyl)-2,3,4,5-tetrahydro-1H-3-benzazepine | (methyloxy)phenyl] | |
| (E167) | acetic acid | |
| 3-Cyclopentyl-7-[4-(3-thienylacetyl)-1-piperazinyl]- | 3-thienylacetic acid | 424 |
| 2,3,4,5-tetrahydro-1H-3-benzazepine (E168) | | |
| 7-[4-(1-Benzothien-4-ylacetyl)-1-piperazinyl]-3- | 1-benzothien-4- | 474 |
| cyclopentyl-2,3,4,5-tetrahydro-1H-3-benzazepine | ylacetic acid | |
| (E169) | | |
| 3-Cyclopentyl-7-(4-{[4-(1- | 4-(1- | 515 |
| piperidinylcarbonyl)phenyl]carbonyl}-1-piperazinyl)- | piperidinylcarbonyl) | |
| 2,3,4,5-tetrahydro-1H-3-benzazepine (E170) | benzoic acid | |
| 7-[4-(1-Benzofuran-4-ylcarbonyl)-1-piperazinyl]-3- | 1-benzofuran-4- | 444 |
| cyclopentyl-2,3,4,5-tetrahydro-1H-3-benzazepine | carboxylic acid | |
| (E171) | | |
| 3-Cyclopentyl-7-[4-(2-furanylcarbonyl)-1-piperazinyl]- | 2-furancarboxylic | 394 |
| 2,3,4,5-tetrahydro-1H-3-benzazepine (E172) | acid | |
| 3-Cyclopentyl-7-{4-[(2,5-dimethyl-3-furanyl)carbonyl]- | 2,5-dimethyl-3- | 422 |
| 1-piperazinyl}-2,3,4,5-tetrahydro-1H-3-benzazepine | furancarboxylic acid | |
| (E173) | | |
| 3-Cyclopentyl-7-[4-(1-methyl-L-prolyl)-1-piperazinyl]- | 1-methyl-L-proline | 411 |
| 2,3,4,5-tetrahydro-1H-3-benzazepine (E174) | | |
| 3-Cyclopentyl-7-{4-[(phenyloxy)acetyl]-1-piperazinyl}- | (phenyloxy)acetic | 434 |
| 2,3,4,5-tetrahydro-1H-3-benzazepine (E175) | acid | |
| 3-Cyclopentyl-7-[4-(2-thienylcarbonyl)-1-piperazinyl]- | 2- | 410 |
| 2,3,4,5-tetrahydro-1H-3-benzazepine (E176) | thiophenecarboxylic | |
| | acid | |
| 3-Cyclopentyl-7-[4-(3-thienylcarbonyl)-1-piperazinyl]- | 3- | 410 |
| 2,3,4,5-tetrahydro-1H-3-benzazepine (E177) | thiophenecarboxylic | |
| | acid | |
| 3-Cyclopentyl-7-{4-[(2,4-dimethyl-1,3-thiazol-5- | (2,4-dimethyl-1,3- | 453 |
| yl)acetyl]-1-piperazinyl}-2,3,4,5-tetrahydro-1H-3- | thiazol-5-yl)acetic | |
| benzazepine (E178) | acid | |
| 3-Cyclopentyl-7-[4-(1,3-thiazol-5-ylcarbonyl)-1- | 1-(1,3-thiazol-5- | 411 |
| piperazinyl]-2,3,4,5-tetrahydro-1H-3-benzazepine | yl)ethanone | |
| (E179) | | |
| 3-Cyclopentyl-7-{4-[(5-phenyl-1,3-thiazol-4- | 5-phenyl-1,3- | 487 |
| yl)carbonyl]-1-piperazinyl}-2,3,4,5-tetrahydro-1H-3- | thiazole-4-carboxylic | |
| benzazepine (E180) | acid | |
| 3-Cyclopentyl-7-[4-(1,3-thiazol-4-ylcarbonyl)-1- | 1,3-thiazole-4- | 411 |
| piperazinyl]-2,3,4,5-tetrahydro-1H-3-benzazepine | carboxylic acid | |
| (E181) | | |
| 3-Cyclopentyl-7-[4-(pyrazolo[1,5-a]pyrimidin-3- | pyrazolo[1,5- | 445 |
| ylcarbonyl)-1-piperazinyl]-2,3,4,5-tetrahydro-1H-3- | a]pyrimidine-3- | |
| benzazepine (E182) | carboxylic acid | |
| 3-Cyclopentyl-7-{4-[(1-methyl-1H-indazol-3- | 1-methyl-1H- | 458 |
| yl)carbonyl]-1-piperazinyl}-2,3,4,5-tetrahydro-1H-3- | indazole-3- | |
| benzazepine (E183) | carboxylic acid | |
| 7-[4-(2,1,3-Benzoxadiazol-5-ylcarbonyl)-1-piperazinyl]- | 2,1,3- | 446 |
| 3-cyclopentyl-2,3,4,5-tetrahydro-1H-3-benzazepine | benzoxadiazole-5- | |
| (E184) | carboxylic acid | |
| 3-Cyclopentyl-7-{4-[(1-methyl-1H-pyrazol-5- | 1-methyl-1H- | 408 |
| yl)carbonyl]-1-piperazinyl}-2,3,4,5-tetrahydro-1H-3- | pyrazole-5- | |
| benzazepine (E185) | carboxylic acid | |
| 3-Cyclopentyl-7-{4-[(1-methyl-3-phenyl-1H-pyrazol-4- | 1-methyl-3-phenyl- | 484 |
| yl)carbonyl]-1-piperazinyl}-2,3,4,5-tetrahydro-1H-3- | 1H-pyrazole-4- | |
| benzazepine (E186) | carboxylic acid | |
| 3-Cyclopentyl-7-{4-[(3,5-dimethyl-4- | 3,5-dimethyl-4- | 423 |
| isoxazolyl)carbonyl]-1-piperazinyl}-2,3,4,5-tetrahydro- | isoxazolecarboxylic | |
| 1H-3-benzazepine (E187) | acid | |
| 3-Cyclopentyl-7-{4-[(4-methyl-1,2,5-oxadiazol-3- | (4-methyl-1,2,5- | 424 |
| yl)acetyl]-1-piperazinyl}-2,3,4,5-tetrahydro-1H-3- | oxadiazol-3-yl)acetic | |
| benzazepine (E188) | acid | |
| 3-Cyclopentyl-7-{4-[(1-methyl-1H-imidazol-2-yl)acetyl]- | (1-methyl-1H- | 422 |
| 1-piperazinyl}-2,3,4,5-tetrahydro-1H-3-benzazepine | imidazol-2-yl)acetic | |
| (E189) | acid | |
| 3-Cyclopentyl-7-{4-[(1-methyl-1H-imidazol-2- | 1-methyl-1H- | 408 |
| yl)carbonyl]-1-piperazinyl}-2,3,4,5-tetrahydro-1H-3- | imidazole-2- | |
| benzazepine (E190) | carboxylic acid | |

### EXAMPLE 191

### 4-(3-Cyclopentyl-2,3,4,5-tetrahydro-1H-3-benzazepin-7-yl)-N-2-thienyl-1-piperazinecarboxamide (E191)

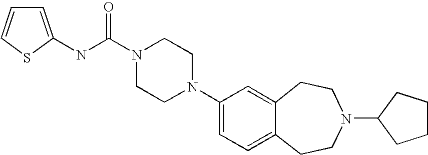

3-Cyclopentyl-7-(1-piperazinyl)-2,3,4,5-tetrahydro-1H-3-benzazepine (D6) (12 mg, 0.04 mmol) was dissolved in dry dichloromethane (0.5 ml) under argon and 2-isocyanatothiophene (6 mg, 0.05 mmol) was added. The solution was stirred at room temperature for 12 hours. Argopore-trisamine resin (4.17 mmol/g, 0.1 g, 0.4 mmol) was added and the reaction mixture stirred for 12 hours. Resin was filtered and washed several times with dichloromethane and the filtrate concentrated in vacuo to afford the title compound; MS (ES+) m/e 425 [M+H]⁺; ¹H NMR (CDCl₃) 7.25 (1H, s), 7.01 (1H, d), 6.99-6.79 (2H, m), 6.70-6.65 (2H, m), 6.57-6.55 (1H, d), 3.65-3.63 (4H, m), 3.19-3.16 (4H, m), 2.90-2.82 (5H, m), 2.70-2.66 (4H, m), 1.86-1.83 (2H, m), 1.68-1.65 (2H, m), 1.55-1.45 (4H, m).

### EXAMPLE 192

### 4-{[4-(3-Cyclobutyl-2,3,4,5-tetrahydro-1H-3-benzazepin-7-yl)-1-piperidinyl]carbonyl}benzonitrile (E192)

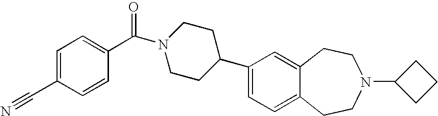

A mixture of 3-cyclobutyl-7-(4-piperidinyl)-2,3,4,5-tetrahydro-1H-3-benzazepine (D12) (100 mg, 0.35 mmol) and polymer bound triethylamine (547 mg, 1.75 mmol) were suspended in dichloromethane (5 ml). The mixture was treated with 4-cyanobenzoyl chloride (70 mg, 0.42 mmol) and stirred at room temperature overnight. The resin was filtered and the filtrate concentrated in vacuo. The residue was purified by flash chromatography, eluting with a gradient of dichloromethane to 1:9:90 0.880 ammonia:ethanol:dichloromethane, to afford the title compound. MS (ES+) m/e 414 [M+H]⁺.

### EXAMPLE 193

### 4-({[(5R)-3-(3-Cyclobutyl-2,3,4,5-tetrahydro-1H-3-benzazepin-7-yl)-2-oxo-1,3-oxazolidin-5-yl]methyl}oxy)benzonitrile (E193)

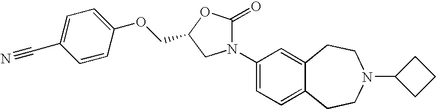

### Step 1: 3-Cyclobutyl-7-nitro-2,3,4,5-tetrahydro-1H-3-benzazepine

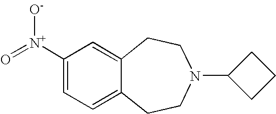

A solution of 7-nitro-2,3,4,5-tetrahydro-1H-3-benzazepine (WO 03/068752) (5.8 g, 30.2 mmol) in dry dichloromethane (200 ml) was treated with cyclobutanone (3.4 ml) and sodium triacetoxyborohydride and stirred at ambient temperature for 1 hour. Saturated sodium hydrogen carbonate solution and dichloromethane were added and the layers separated. The organic layer was washed with saturated sodium hydrogen carbonate solution, water and saturated brine, dried (MgSO₄) and concentrated in vacuo to afford the title compound. MS (ES+) m/e 247 [M+H]⁺.

### Step 2: 3-Cyclobutyl-2,3,4,5-tetrahydro-1H-3-benzazepin-7-amine

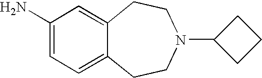

A solution of 3-cyclobutyl-7-nitro-2,3,4,5-tetrahydro-1H-3-benzazepine (product of E193, step 1) (6.8 g, 27.6 mmol) in methanol (60 ml) and tetrahydrofuran (90 ml) was hydrogenated overnight in the presence of 10% palladium on carbon paste. After filtration of the catalyst through Kieselguhr, the filtrate was concentrated in vacuo to afford the title compound. MS (ES+) m/e 217 [M+H]⁺.

### Step 3: Phenylmethyl (3-cyclobutyl-2,3,4,5-tetrahydro-1H-3-benzazepin-7-yl)carbamate

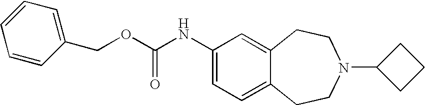

A solution of 3-cyclobutyl-2,3,4,5-tetrahydro-1H-3-benzazepin-7-amine (product of E193, step 2) (1.0 g, 4.6 mmol) in acetone (20 ml) and water (5 ml) was treated with sodium hydrogen carbonate (1.1 g, 12.7 mmol) and benzyl chloroformate (0.78 ml, 5.5 mmol) and stirred at ambient temperature for 1 hour. The reaction mixture was diluted with ethyl acetate, washed with water and brine, dried (MgSO₄) and concentrated in vacuo. The residue was purified by column chromatography eluting with 0:1-1:4 methanol/ethyl acetate to afford the title compound. MS (ES+) m/e 351 [M+H]⁺.

### Step 4: (5R)-3-(3-Cyclobutyl-2,3,4,5-tetrahydro-1H-3-benzazepin-7-yl)-5-(hydroxymethyl)-1,3-oxazolidin-2-one

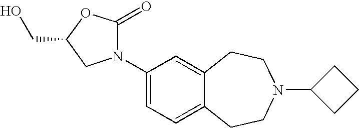

The title compound was prepared from phenylmethyl (3-cyclobutyl-2,3,4,5-tetrahydro-1H-3-benzazepin-7-yl)carbamate (product of E193, step 3) using the method described in WO 02/059115; MS (ES+) m/e 317 [M+H]⁺.

### Step 5: [(5R)-3-(3-Cyclobutyl-2,3,4,5-tetrahydro-1H-3-benzazepin-7-yl)-2-oxo-1,3-oxazolidin-5-yl]methyl methanesulfonate

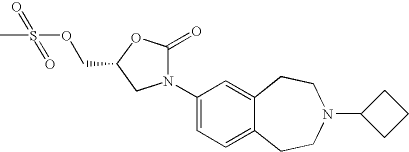

A solution of (5R)-3-(3-cyclobutyl-2,3,4,5-tetrahydro-1H-3-benzazepin-7-yl)-5-(hydroxymethyl)-1,3-oxazolidin-2-one (product of E193, step 4) (0.40 g, 1.3 mmol) in dry dichloromethane (5 ml) was treated with triethylamine (0.19 ml, 1.4 mmol) followed by methanesulphonyl chloride (0.11 ml, 1.4 mmol) and stirred at ambient temperature for 1.5 hours. The reaction mixture was diluted with dichloromethane, washed with saturated sodium hydrogen carbonate solution, dried (MgSO4) and concentrated in vacuo to afford the title compound. MS (ES+) m/e 395 [M+H]⁺.

### Step 6: 4-({[(5R)-3-(3-Cyclobutyl-2,3,4,5-tetrahydro-1H-3-benzazepin-7-yl)-2-oxo-1,3-oxazolidin-5-yl]methyl}oxy)benzonitrile

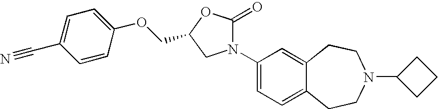

A solution of 4-cyanophenol (0.058 g, 0.49 mmole) in dry dimethylformamide (5 ml) was treated with 60% sodium hydride in mineral oil (0.02 g, 0.51 mmole) and stirred for 0.5 hours at ambient temperature. [(5R)-3-(3-Cyclobutyl-2,3,4,5-tetrahydro-1H-3-benzazepin-7-yl)-2-oxo-1,3-oxazolidin-5-yl]methyl methanesulfonate (product of E193, step 5) (0.2 g, 0.51 mmol) was added and the mixture stirred for 18 hours at 100° C. After cooling to ambient temperature, the reaction mixture was applied to a SCX ion exchange cartridge (Varian bond-elute) and washed with methanol and then 2M 0.880 ammonia/methanol. The basic fractions were concentrated in vacuo. The residue was purified by column chromatography eluting with dichloromethane/methanol (1:0-9:1) to afford the title compound. MS (ES+) m/e 418 [M+H]⁺.

### EXAMPLE 194

### 4-(3-Cyclobutyl-2,3,4,5-tetrahydro-1H-3-benzazepin-7-yl)benzonitrile (E194)

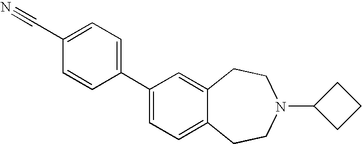

### Step 1:1,1-Dimethylethyl 7-(4,4,5,5-tetramethyl--1,3,2-dioxaborolan-2-yl)-1,2,4,5-tetrahydro-3H-3-benzazepine-3-carboxylate

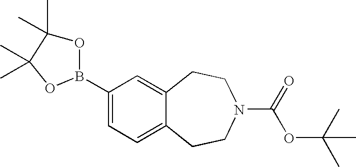

A mixture of 1,1-dimethylethyl-7-{[(trifluoromethyl)sulfonyl]oxy}-1,2,4,5-tetrahydro-3H-3-benzazepine-3-carboxylate (D1; Bioorganic and Medicinal Chemistry Letters (2000), 10(22), 2553-2555) (250 mg, 0.63 mmol), 4,4,4′,4′,5,5,5′,5′-octamethyl-2,2′-bi-1,3,2-dioxaborolane (176 mg, 0.70 mmol), 1,1′bis(diphenylphosphino)ferrocene dichloropalladium (II) complex (14 mg, 0.02 mmol), 1,1′bis(diphenylphosphino)ferrocene (11 mg, 0.02 mmol) and potassium acetate (186 mg, 2.00 mmol) in dioxan (5 ml) were heated in a microwave reactor at 140° C. for 600 seconds at 200 W. The mixture was diluted with ethyl acetate, washed with water (×3), then brine and dried over magnesium sulphate. The residue was purified by column chromatography eluting ethyl acetate/hexane (1:4) to afford the title compound. MS (ES+) m/e 274. [M+H−100]⁺ (loss of carboxylate group).

### Step 2: 1,1-Dimethylethyl 7-(4-cyanophenyl)-1,2,4,5-tetrahydro-3H-3-benzazepine-3-carboxylate

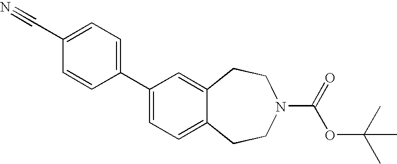

1,1-Dimethylethyl 7-(4,4,5,5-tetramethyl-1,3,2-dioxaborolan-2-yl)-1,2,4,5-tetrahydro-3H-3-benzazepine-3-carboxylate (product of E194, step 1) (180 mg, 0.48 mmol), 4-bromobenzonitrile (97 mg, 0.53 mmol), tetrakistriphenylphosphine palladium 17 mg, 0.015 mmol), sodium carbonate (102 mg, 0.97 mmol) and 1,2-dimethoxyethane/water/ethanol 7:3:1 (5 ml) were heated in a microwave reactor at 160° C. for 900 seconds at 200 W. The mixture was diluted with ethyl acetate, washed with water (×3), then brine and dried over magnesium sulphate. The residue was purified by column chromatography eluting ethyl acetate/hexane (1:4) to afford the title compound. MS (ES+) m/e 249. [M+H−100]⁺ (loss of carboxylate group).

### Step 3: 4-(2,3,4,5-Tetrahydro-1H-3-benzazepin-7-yl)benzonitrile

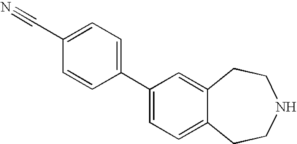

1,1-Dimethylethyl 7-(4-cyanophenyl)-1,2,4,5-tetrahydro-3H-3-benzazepine-3-carboxylate (product of E194, step 2) (203 mg, 0.58 mmol) was dissolved in dioxan (3 ml) and hydrochloric acid in dioxan (4M; 5 ml) was added. The reaction was stirred at room temperature for 24 hours. Solvent was then removed in vacuo and the product was dissolved in methanol and applied to a SCX cartridge (Varian bond-elute, 5 g) and washed with methanol and then a mixture of 2M ammonia/methanol. The basic fractions were combined and solvent was removed in vacuo to afford the title compound. MS (ES+) m/e 249 [M+H]⁺.

### Step 4: 4-(3-Cyclobutyl-2,3,4,5-tetrahydro-1H-3-benzazepin-7-yl)benzonitrile

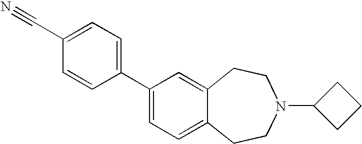

4-(2,3,4,5-Tetrahydro-1H-3-benzazepin-7-yl)benzonitrile (product of E194, step 3)(85 mg, 0.34 mmol), cyclobutanone (0.05 ml, 0.68 mmol), sodium triacetoxyborohydride (145 mg, 0.68 mmol) 4 molecular sieves (50 mg) and dichloromethane (5 ml) were stirred at room temperature for 2 hours. The reaction mixture was filtered and solvent was removed in vacuo. The product was dissolved in methanol and applied to a SCX cartridge (Varian bond-elute, 5 g) and washed with methanol and then a mixture of 2M ammonia/methanol. The basic fractions were combined and solvent was removed in vacuo to afford the title compound. MS (ES+) m/e 303 [M+H]⁺.

### EXAMPLE 195

### 4-(3-Cyclobutyl-2,3,4,5-tetrahydro-1H-3-benzazepin-7-yl)-N-methylbenzamide (E195)

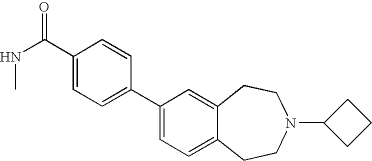

Example 195 was prepared using an analogous method to that described for Example 194 (steps 2-4) from 1,1′-dimethylethyl 7-(4,4,5,5-tetramethyl-1,3,2-dioxaborolan-2-yl)-1,2,4,5-tetrahydro-3H-3-benzazepine-3-carboxylate (product of Example E194, step 1) and 4-bromo-N-methylbenzamide (WO 03/068749A1). MS (ES+) m/e 335 [M+H]⁺.

### EXAMPLE 196

### 1-[4-(3-Cyclobutyl-2,3,4,5-tetrahydro-1H-3-benzazepin-7-yl) phenyl]-2-propanone

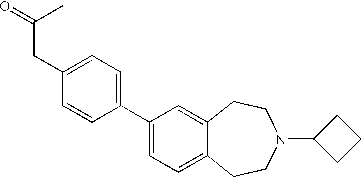

### Step 1: 3-Cyclobutyl-2,3,4,5-tetrahydro-1H-3-benzazepin-7-yl trifluoromethanesulfonate

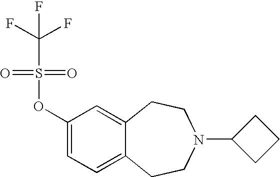

Step 1 was carried out using an analogous method to that described for Example 194 steps 3-4 using 1,1-dimethylethyl-7-{[(trifluoromethyl)sulfonyl]oxy}-1,2,4,5-tetrahydro-3H-3-benzazepine-3-carboxylate (D1) to afford the title compound. MS (ES+) m/e 350. [M+H]⁺.

### Step 2: 3-cyclobutyl-7-(4,4,5,5-tetramethyl-1,3,2-dioxaborolan-2-yl)-2,3,4,5-tetrahydro-1H-3-benzazepine

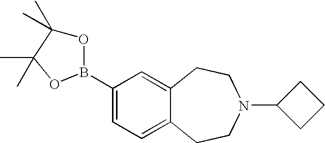

Step 2 was carried out using an analogous method to that described for Example 194 step 1 using 3-cyclobutyl-2,3,4,5-tetrahydro-1H-3-benzazepin-7-yl trifluoromethanesulfonate (product of E196, step 1) to afford the title compound. MS (ES+) m/e 328. [M+H]⁺.

### Step 3: 1-[4-(3-Cyclobutyl-2,3,4,5-tetrahydro-1H-3-benzazepin-7-yl)phenyl]-2-propanone

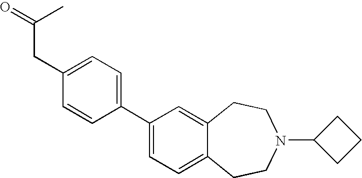

Step 3 was carried out using an analogous method to that described for Example 194 step 2 using 3-cyclobutyl-7-(4,4,5,5-tetramethyl-1,3,2-dioxaborolan-2-yl)-2,3,4,5-tetrahydro-1H-3-benzazepine (product of E196, step 2) (135 mg, 0.41 mmol) and 1-(4-bromophenyl)-2-propanone (97 mg, 0.45 mmol) to afford the title compound. MS (ES+) m/e 334. [M+H]⁺.

### EXAMPLE 197

### 2-[4-(3-Cyclobutyl-2,3,4,5-tetrahydro-1H-3-benzazepin-7-yl)phenyl]-N-methylacetamide (E197)

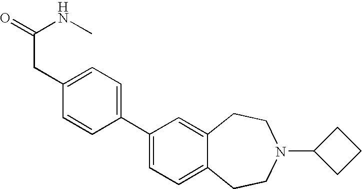

### Step 1: 1,1-Dimethylethyl 7-{4-[2-(methylamino)-2-oxoethyl]phenyl}-1,2,4,5-tetrahydro-3H-3-benzazepine-3-carboxylate

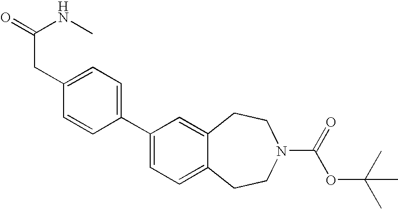

1,1-dimethylethyl 7-(4,4,5,5-tetramethyl-1,3,2-dioxaborolan-2-yl)-1,2,4,5-tetrahydro-3H-3-benzazepine-3-carboxylate (product of Example E194, Step 1) (379 mg, 1.02 mmol), 2-(4-bromophenyl)-N-methylacetamide (Tetrahedron (1966), 22(9), 2995-9) (255 mg, 1.18 mmol), tetrakistriphenylphosphine palladium (35 mg, 0.030 mmol), sodium carbonate (3.3 ml, 2M) and 1,2-dimethoxyethane (10 ml)) were heated at 80° C. for 16 hours. The mixture was diluted with ethyl acetate, washed with water (×3), then brine and dried over magnesium sulphate. The residue was purified by column chromatography eluting 0-10% (2M ammonia in methanol/dichloromethane) to afford the title compound. MS (ES+) m/e 295. [M+H−100]⁺ (loss of carboxylate group).

### Step 2: 2-[4-(3-Cyclobutyl-2,3,4,5-tetrahydro-1H-3-benzazepin-7-yl)phenyl]-N-methylacetamide

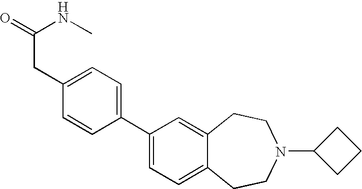

Step 2 was carried out using an analogous method to that described for Example 194 steps 3-4 using 1,1-dimethylethyl 7-{4-[2-(methylamino)-2-oxoethyl]phenyl}-1,2,4,5-tetrahydro-3H-3-benzazepine-3-carboxylate (product of E197, step 1). MS (ES+) m/e 349. [M+H]⁺

### EXAMPLE 198

### 6-(3-Cyclobutyl-2,3,4,5-tetrahydro-1H-3-benzazepin-7-yl)-N-methyl-3-pyridinecarboxamide (E198)

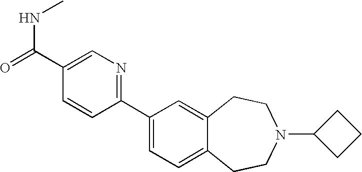

### Step 1: 1,1-Dimethylethyl 7-{5-[(methyloxy)carbonyl]-2-pyridinyl}-1,2,4,5-tetrahydro-3H-3-benzazepine-3-carboxylate

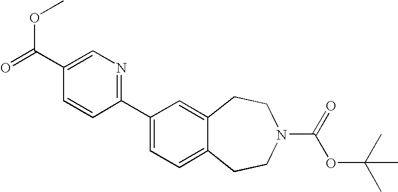

Step 1 was carried out using an analogous method to that described for Example 197 step 1 using 1,1-dimethylethyl 7-(4,4,5,5-tetramethyl-1,3,2-dioxaborolan-2-yl)-1,2,4,5-tetrahydro-3H-3-benzazepine-3-carboxylate (product of Example E194, Step 1) (888 mg, 2.38 mg), and methyl 6-chloro-3-pyridinecarboxylate (449 mg, 2.62 mmol).

¹H NMR (400 MHz) CDCl₃ 69.26 (1H (s) CH—Ar), δ8.34 (1H (d) CH—Ar), δ7.86 (1H (s) CH—Ar), δ7.80 (2H (d) CH—Ar), δ97.23 (1H (s) CH—Ar), δ3.97 (3H (s) CH₃), δ3.59 (4H (m) 2×CH₂), δ3.00 (4H (m) 2×CH₂), δ1.49 (9H (s) 3×CH₃).

### Step 2: Methyl 6-(2,3,4,5-tetrahydro-1H-3-benzazepin-7-yl)-3-pyridinecarboxylate

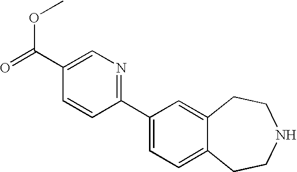

1,1-Dimethylethyl 7-{5-[(methyloxy)carbonyl]-2-pyridinyl}-1,2,4,5-tetrahydro-3H-3-benzazepine-3-carboxylate (product of E198, step 1) (495 mg, 1.8 mmol), was dissolved in dichloromethane (10 ml), and the mixture was cooled to 0° C. Trifluoroacetic acid (3 ml) was slowly added and the mixture was warmed to room temperature and stirred for 30 minutes. Solvent was removed in vacuo and the residue was dissolved in methanol, then applied to a SCX cartridge (Varian bond-elute, 10 g) and washed with methanol and then a mixture of 2M ammonia/methanol. The basic fractions were combined and solvent was removed in vacuo to afford the title compound. MS (ES+) m/e 283 [M+H]⁺.

### Step 3: Methyl 6-(3-cyclobutyl-2,3,4,5-tetrahydro-1H-3-benzazepin-7-yl)-3-pyridinecarboxylate

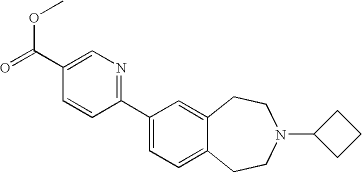

Step 3 was carried out using an analogous method to that described for Example 194 step 4 using methyl 6-(2,3,4,5-tetrahydro-1H-3-benzazepin-7-yl)-3-pyridinecarboxylate (product of E198, step 2). MS (ES+) m/e 337 [M+H]⁺.

### Step 4: 6-(3-Cyclobutyl-2,3,4,5-tetrahydro-1H-3-benzazepin-7-yl)-3-pyridinecarboxylic acid

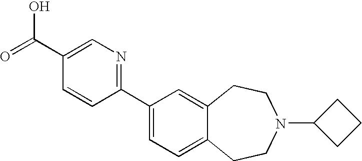

Methyl 6-(3-cyclobutyl-2,3,4,5-tetrahydro-1H-3-benzazepin-7-yl)-3-pyridinecarboxylate (product of E198, step 3) (306 mg, 0.91 mmol), was dissolved in methanol (10 ml) and lithium hydroxide (36 mg, dissolved in 5 ml water) was added. The mixture was stirred at room temperature for 24 hours. The solvent was removed in vacuo and the residue was azeotroped with ether to afford the title compound. MS (ES+) m/e 323 [M+H]⁺

### Step 5: 6-(3-Cyclobutyl-2,3,4,5-tetrahydro-1H-3-benzazepin-7-yl)-N-methyl-3-pyridinecarboxamide

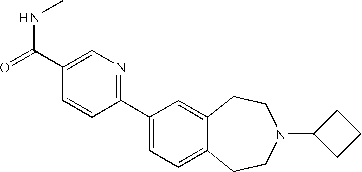

6-(3-Cyclobutyl-2,3,4,5-tetrahydro-1H-3-benzazepin-7-yl)-3-pyridinecarboxylic acid (product of E198, step 4) (180 mg, 0.56 mmol), methylamine (2M in tetrahydrofuran (2.7 ml), HATU (206 mg, 0.67 mmol), triethylamine (0.2 ml, 1.34 mmol) and N,N-dimethylformamide (5 ml) were stirred at room temperature for 16 hours. Solvent was removed in vacuo and the residue was dissolved in methanol. It was applied to a SCX cartridge (Varian bond-elute, 10 g) and washed with methanol and then a mixture of 2M ammonia/methanol. The basic fractions were collected and the product was purified further by column chromatography eluting 0-10% (2M ammonia in methanol/dichloromethane) to afford the title compound. MS (ES+) m/e 336. [M+H]⁺.

### EXAMPLE 199

### 3-Cyclobutyl-7-[5-(4-morpholinylcarbonyl)-2-pyridinyl]-2,3,4,5-tetrahydro-1H-3-benzazepine (E199)

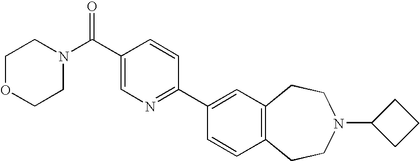

Example 199 was prepared using an analogous method to that described for Example 198 step 5 from 6-(3-cyclobutyl-2,3,4,5-tetrahydro-1H-3-benzazepin-7-yl)-3-pyridinecarboxylic acid (product of Example E198, Step 4) and morpholine, MS (ES+) m/e 392. [M+H]⁺.

### EXAMPLE 200

### 3-(3-Cyclobutyl-2,3,4,5-tetrahydro-1H-3-benzazepin-7-yl)-N-methylbenzamide (E200)

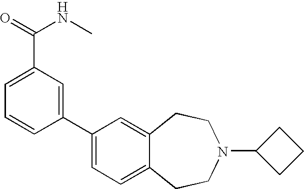

### Step 1: 1,1-Dimethylethyl 7-{3-[(methylamino)carbonyl]phenyl}-1,2,4,5-tetrahydro-3H-3-benzazepine-3-carboxylate

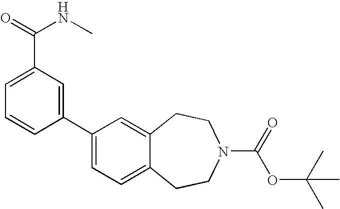

Step 1 was carried out using an analogous method to that described for Example 197 step 1 using 1,1-dimethylethyl 7-(4,4,5,5-tetramethyl-1,3,2-dioxaborolan-2-yl)-1,2,4,5-tetrahydro-3H-3-benzazepine-3-carboxylate (product of Example E194, Step 1) (300 mg, 0.80 mmol) and 3-bromo-N-methylbenzamide (189 mg, 0.88 mmol). ¹H NMR (400 MHz) CDCl_{3 δ}7.98 (1H (s) CH—Ar), δ7.70 (2H (m) CH—Ar), δ7.49 (1H (t) CH—Ar), δ7.37 (2H (m) CH—Ar), δ7.21 (1H (s) CH—Ar), δ6.21 (1H (s) N—H), δ3.56 (4H (m) 2×CH₂), δ3.05 (3H (d) CH₃), δ2.95 (4H (m) 2×CH₂), δ1.49 (9H (s) 3×CH₃).

### Step 2: 3-(3-Cyclobutyl-2,3,4,5-tetrahydro-1H-3-benzazepin-7-yl)-N-methylbenzamide

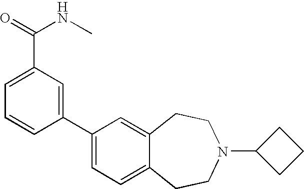

Step 2 was carried out using an analogous method to that described for Example 98 steps 2-3 using 1,1-dimethylethyl 7-{3-[(methylamino)carbonyl]phenyl}-1,2,4,5-tetrahydro-3H-3-benzazepine-3-carboxylate (product of E200, step 1); MS (ES+) m/e 335. [M+H]⁺.

### EXAMPLE 201-204 (E201-204)

Examples 201-204 were prepared using an analogous method to that described for Example 200 steps 1-2 from 1,1-dimethylethyl 7-(4,4,5,5-tetramethyl-1,3,2-dioxaborolan-2-yl)-1,2,4,5-tetrahydro-3H-3-benzazepine-3-carboxylate (product of Example E194, Step 1) and the appropriate halide indicated in the table below.

| | | LC/MS |
|---|---|---|
| Example | Halide | (M + H⁺⁾ |
| 5-(3-Cyclobutyl-2,3,4,5-tetrahydro-1H-3- | 5-bromo-2- | 293 |
| benzazepin-7-yl)-2-furancarbonitrile | furancarbonitrile | |
| (E201) | | |
| 3-Cyclobutyl-7-(1,3-thiazol-2-yl)-2,3,4,5- | 2-bromo-1,3-thiazole | 285 |
| tetrahydro-1H-3-benzazepine | | |
| (E202) | | |
| 4-(3-Cyclobutyl-2,3,4,5-tetrahydro-1H-3- | 4-bromo-N,3-dimethyl | 353 |
| benzazepin-7-yl)-N,3-dimethylbenzamide | benzamide (PCT Int. Appl. | |
| (E203) | (1995), 19 pp. WO | |
| | 9526328 A1) | |
| 4-(3-Cyclobutyl-2,3,4,5-tetrahydro-1H-3- | 4-bromo-3-fluoro-N- | 349 |
| benzazepin-7-yl)-3-fluoro-N-methylbenzamide | methylbenzamide (D8) | |
| (E204) | | |

### EXAMPLE 205

### 6-{[4-(3-Cyclobutyl-2,3,4,5-tetrahydro-1H-3-benzazepin-7-yl)phenyl]oxy}-N-methyl-3-pyridinecarboxamide

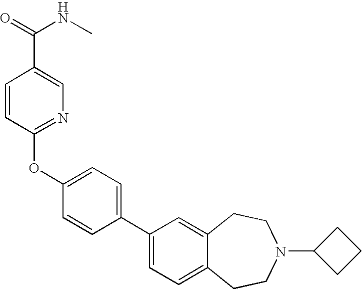

### Step 1: 1,1-Dimethylethyl 7-(4-hydroxyphenyl)-1,2,4,5-tetrahydro-3H-3-benzazepine-3-carboxylate

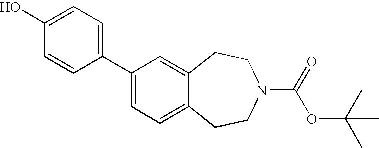

Step 1 was carried out using an analogous method to that described for Example 197 step 1 using 1,1-dimethylethyl 7-(4,4,5,5-tetramethyl-1,3,2-dioxaborolan-2-yl)-1,2,4,5-tetrahydro-3H-3-benzazepine-3-carboxylate (product of Example E194, Step 1) (1 g, 2.68 mmol) and 4-bromophenol (556 mg, 3.21 mmol). To afford the title compound. MS (ES+) m/e 340 [M+H−100]⁺.

### Step 2: 1,1-Dimethylethyl 7-[4-({5-[(methylamino)carbonyl]-2-pyridinyl}oxy)phenyl]-1,2,4,5-tetrahydro-3H-3-benzazepine-3-carboxylate

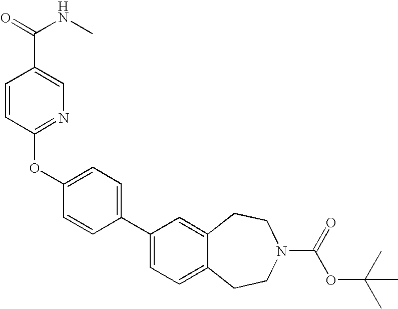

1,1-Dimethylethyl 7-(4-hydroxyphenyl)-1,2,4,5-tetrahydro-3H-3-benzazepine-3-carboxylate (product of E205, step 1) (120 mg, 0.35 mmol) was dissolved in dimethylsulfoxide (10 ml) and cooled to 0° C. Sodium hydride (25 mg, 1.06 mmol) was then added and the mixture was stirred for 30 minutes at 0° C. 6-chloro-N-methyl-3-pyridinecarboxamide (PCT Int. Appl. (2002), WO 2002046186)(181 mg, 1.06 mmol) was then added and the mixture was heated at 120° C. for 48 hours. The reaction mixture was cooled to room temperature and poured onto ice/water, it was extracted into dichloromethane (×3), washed with water, then brine and dried using sodium sulphate. The product was purified by column chromatography eluting 5-20% (ethylacetate/hexane) to afford the title compound. MS (ES+) m/e 374 [M+H−100]+(loss of carboxylate group).

### Step 3: 6-{[4-(3-Cyclobutyl-2,3,4,5-tetrahydro-1H-3-benzazepin-7-yl)phenyl]oxy}-N-methyl-3-pyridinecarboxamide

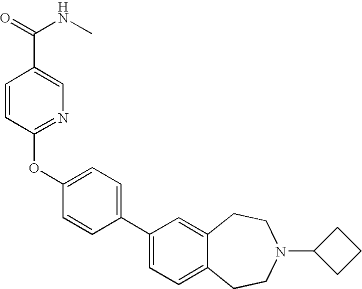

Step 3 was carried out using an analogous method to that described for Example 198 steps 2-3 using 1,1-dimethylethyl 7-[4-({5-[(methylamino)carbonyl]-2-pyridinyl}oxy)phenyl]-1,2,4,5-tetrahydro-3H-3-benzazepine-3-carboxylate (product of E205, step 2) to afford the title compound. MS (ES+) m/e 428 [M+H]⁺.

### EXAMPLE 206

### 3-cyclobutyl-7-[1-(tetrahydro-2H-pyran-4-ylcarbonyl)-4-piperidinyl]-2,3,4,5-tetrahydro-1 H-3-benzazepine (E206)

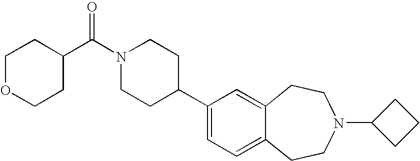

A mixture of tetrahydro-2H-pyran-4-carboxylic acid (62 mg, 0.48 mmol), N-Cyclohexylcarbodiimide N′-methyl polystyrene (282 mg, 0.4 mmol), and 1-hydroxybenzotriazole (65 mg, 0.48 mmol) in dry dimethylformamide (2 ml) were stirred under argon at room temperature for 60 minutes. A solution of 3-cyclobutyl-7-(4-piperidinyl)-2,3,4,5-tetrahydro-1H-3-benzazepine (D12) (69 mg, 0.24 mmol) in dry dimethylformamide (0.5 ml) was added, and the reaction mixture left to stir at room temperature for one day. The mixture was applied to a SCX ion exchange cartridge (Varian bond-elute, 5 g), washed with methanol and then with a 2M ammonia in methanol solution. The combined basic fractions were concentrated in vacuo and the resulting residue purified by column chromatography eluting with a mixture of 2M ammonia in methanol and dichloromethane (1-2%) to afford the title product; MS (ES+) m/e 397 [M+H]⁺.

### EXAMPLES 207-220 (E207-220)

Examples 207-220 (E207-E220) were prepared using an analogous method to that described for Example 206 (E206) from 3-cyclobutyl-7-(4-piperidinyl)-2,3,4,5-tetrahydro-1H-3-benzazepine (D12) and the appropriate carboxylic acid as indicated in the table.

| | | LC/MS |
|---|---|---|
| Example | Acid | (M + H⁺) |
| 5-{[4-(3-cyclobutyl-2,3,4,5-tetrahydro-1H-3- | 6-cyano-3- | 415 |
| benzazepin-7-yl)-1-piperidinyl]carbonyl}-2- | pyridinecarboxylic acid | |
| pyridinecarbonitrile (E207) | | |
| 3-{[4-(3-cyclobutyl-2,3,4,5-tetrahydro-1H-3- | 3-cyanobenzoic acid | 413 |
| benzazepin-7-yl)-1-piperidinyl]carbonyl}benzonitrile | | |
| (E208) | | |
| 3-cyclobutyl-7-[1-(2-pyrazinylcarbonyl)-4- | 2-pyrazinecarboxylic | 391 |
| piperidinyl]-2,3,4,5-tetrahydro-1H-3-benzazepine | acid | |
| (E209) | | |
| 3-cyclobutyl-7-{1-[(4-fluorophenyl)carbonyl]-4- | 4-fluorobenzoic acid | 407 |
| piperidinyl}-2,3,4,5-tetrahydro-1H-3-benzazepine | | |
| (E210) | | |
| 7-[1-(2,1,3-benzoxadiazol-5-ylcarbonyl)-4- | 2,1,3-benzoxadiazole- | 431 |
| piperidinyl]-3-cyclobutyl-2,3,4,5-tetrahydro-1H-3- | 5-carboxylic acid | |
| benzazepine (E211) | | |
| 3-cyclobutyl-7-(1-{[6-(trifluoromethyl)-3- | 6-(trifluoromethyl)-3- | 458 |
| pyridinyl]carbonyl}-4-piperidinyl)-2,3,4,5-tetrahydro- | pyridinecarboxylic acid | |
| 1H-3-benzazepine (E212) | | |
| 3-cyclobutyl-7-[1-(1,3-thiazol-4-ylcarbonyl)-4- | 1,3-thiazole-4- | 396 |
| piperidinyl]-2,3,4,5-tetrahydro-1H-3-benzazepine | carboxylic acid | |
| (E213) | | |
| 3-cyclobutyl-7-{1-[(2-fluorophenyl)carbonyl]-4- | 2-fluorobenzoic acid | 407 |
| piperidinyl}-2,3,4,5-tetrahydro-1H-3-benzazepine | | |
| (E214) | | |
| 3-cyclobutyl-7-[1-(2-pyridinylcarbonyl)-4-piperidinyl]- | 2-pyridinecarboxylic | 390 |
| 2,3,4,5-tetrahydro-1H-3-benzazepine (E215) | acid | |
| 3-cyclobutyl-7-[1-(3-pyridinylcarbonyl)-4-piperidinyl]- | 3-pyridinecarboxylic | 390 |
| 2,3,4,5-tetrahydro-1H-3-benzazepine (E216) | acid | |
| 3-cyclobutyl-7-[1-(pyrazolo[1,5-a]pyrimidin-3- | pyrazolo[1,5- | 430 |
| ylcarbonyl)-4-piperidinyl]-2,3,4,5-tetrahydro-1H-3- | a]pyrimidine-3- | |
| benzazepine (E217) | carboxylic acid | |
| 3-cyclobutyl-7-[1-(6-quinoxalinylcarbonyl)-4- | 6- | 441 |
| piperidinyl]-2,3,4,5-tetrahydro-1H-3-benzazepine | quinoxalinecarboxylic | |
| (E218) | acid | |
| 3-cyclobutyl-7-[1-(5-quinoxalinylcarbonyl)-4- | 5- | 441 |
| piperidinyl]-2,3,4,5-tetrahydro-1H-3-benzazepine | quinoxalinecarboxylic | |
| (E219) | acid | |
| 3-cyclobutyl-7-{1-[(6-methyl-3-pyridinyl)carbonyl]-4- | 6-methyl-3- | 404 |
| piperidinyl}-2,3,4,5-tetrahydro-1H-3-benzazepine | pyridinecarboxylic acid | |
| (E220) | | |

### EXAMPLE 221

### 3-cyclobutyl-7-[1-(6-methyl-3-pyridinyl)-4-piperidinyl]-2,3,4,5-tetrahydro-1H-3-benzazepine (E221)

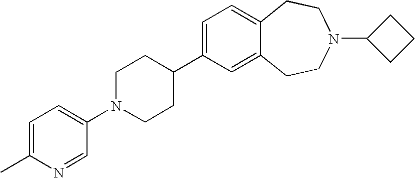

3-Cyclobutyl-7-(4-piperidinyl)-2,3,4,5-tetrahydro-1H-3-benzazepine (D12) (141 mg, 0.50 mmol), 5-bromo-2-methylpyridine (109 mg, 0.63 mmol), sodium tert-butoxide (34 mg, 0.98 mmol), tris(dibenzylideneacetone)dipalladium(0) (26 mg, 0.04 mmol) and 2′-(dicyclohexylphosphanyl)-N,N-dimethyl-2-biphenylamine (32 mg, 0.11 mmol) were mixed in 4 ml of dry 1,4-dioxan. The reaction mixture was heated in microwave at 120° C. for 10 min. The mixture was applied to a SCX ion exchange cartridge (Varian bond-elute, 5 g), washed with methanol and then with a 2M ammonia in methanol solution. The combined basic fractions were concentrated in vacuo and the resulting residue purified by column chromatography eluting with a mixture of 2M ammonia in methanol and dichloromethane (4%) to give a yellow solid which was triturated with diethyl ether to afford the title product; MS (ES+) m/e 376 [M+H]⁺.

### EXAMPLE 222

### 3-cyclobutyl-7-{1-[6-(trifluoromethyl)-3-pyridinyl]-4-piperidinyl}-2,3,4,5-tetrahydro-1H-3-benzazepine (E222)

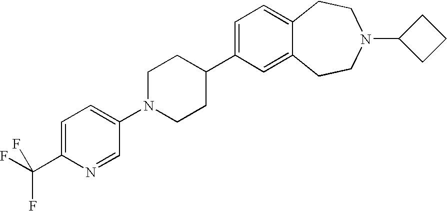

The title compound was prepared from 3-Cyclobutyl-7-(4-piperidinyl)-2,3,4,5-tetrahydro-1H-3-benzazepine (D12) and 5-bromo-2-(trifluoromethyl)pyridine using the same method described for the preparation of Example 221. MS (ES+) m/e 430 [M+H]⁺.

### EXAMPLE 223

### 3-[4-(3-cyclobutyl-2,3,4,5-tetrahydro-1H-3-benzazepin-7-yl)-1-piperidinyl]benzonitrile (E223)

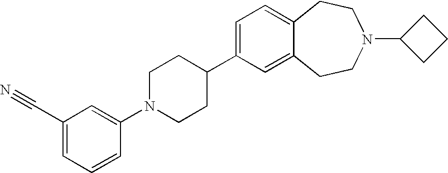

3-Cyclobutyl-7-(4-piperidinyl)-2,3,4,5-tetrahydro-1H-3-benzazepine (D12) (160 mg, 0.56 mmol), 3-bromobenzonitrile (108 mg, 0.59 mmol), cesium carbonate (255 mg, 0.79 mmol), palladium acetate (7 mg, 0.03 mmol) and (9,9-dimethyl-9H-xanthene-4,5-diyl)bis(diphenylphosphane) (26 mg, 0.04 mmol) were mixed in 2.5 ml of toluene. The reaction mixture was heated in microwave at 140° C. for 60 minutes. Ethyl acetate was added and the mixture filtered through celite, washed with brine, dried over anhydrous magnesium sulfate and concentrated in vacuo. The crude residue was purified by column chromatography eluting with a mixture of 2M ammonia in methanol and dichloromethane (1-2%) to afford the title product; MS (ES+) m/e 386 [M+H]⁺.

### EXAMPLE 224

### 4-[4-(3-cyclobutyl-2,3,4,5-tetrahydro-1H-3-benzazepin-7-yl)-1-piperidinyl]benzonitrile (E224)

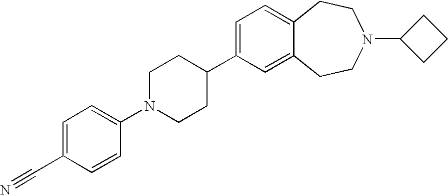

3-Cyclobutyl-7-(4-piperidinyl)-2,3,4,5-tetrahydro-1H-3-benzazepine (D12) (143 mg, 0.50 mmol), 4-bromobenzonitrile (109 mg, 0.60 mmol), cesium carbonate (249 mg, 0.76 mmol), palladium acetate (12 mg, 0.05 mmol) and (9,9-dimethyl-9H-xanthene-4,5-diyl)bis(diphenylphosphane) (26 mg, 0.04 mmol) were mixed in 1.5 ml of toluene and 1 ml of acetonitrile. The reaction mixture was heated in microwave at 140° C. for 120 minutes. The mixture filtered through celite and applied to a SCX ion exchange cartridge (Varian bond-elute, 5 g), washed with methanol and then with a 2M ammonia in methanol solution. The combined basic fractions were concentrated in vacuo and the resulting residue purified by column chromatography eluting with a mixture of 2M ammonia in methanol and dichloromethane (3-5%) to afford the title product; MS (ES+) m/e 386 [M+H]⁺.

### EXAMPLE 225

### 6-[4-(3-cyclobutyl-2,3,4,5-tetrahydro-1H-3-benzazepin-7-yl)-1-piperidinyl]-N-methyl-3-pyridinecarboxamide (E225)

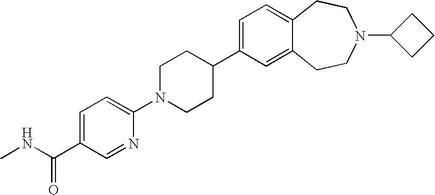

3-Cyclobutyl-7-(4-piperidinyl)-2,3,4,5-tetrahydro-1H-3-benzazepine (D12) (100 mg, 0.35 mmol), 6-chloro-N-methyl-3-pyridinecarboxamide (PCT Int. Appl. (2002), WO 2002046186) (72 mg, 0.42 mmol) and potassium carbonate (107 mg, 0.77 mmol) were mixed in 2 ml of 1-methyl-2-pyrrolidinone. The reaction mixture was heated in microwave at 210° C. for 30 minutes. The mixture was applied to a SCX ion exchange cartridge (Varian bond-elute, 5 g), washed with methanol and then with a 2M ammonia in methanol solution. The combined basic fractions were concentrated in vacuo and the resulting residue purified by column chromatography eluting with a mixture of 2M ammonia in methanol and dichloromethane (1-3%) to afford the title product; MS (ES+) m/e 419 [M+H]⁺.

### EXAMPLE 226

### 5-[4-(3-cyclobutyl-2,3,4,5-tetrahydro-1H-3-benzazepin-7-yl)-1-piperidinyl]-N-methyl-2-pyrazinecarboxamide (E226)

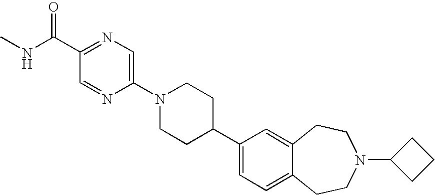

### Step 1: methyl 5-[4-(3-cyclobutyl-2,3,4,5-tetrahydro-1H-3-benzazepin-7-yl)-1-piperidinyl]-2-pyrazinecarboxylate

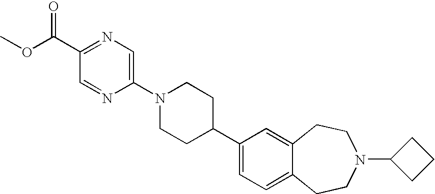

3-Cyclobutyl-7-(4-piperidinyl)-2,3,4,5-tetrahydro-1H-3-benzazepine (D12) (180 mg, 0.63 mmol), methyl 5-chloro-2-pyrazinecarboxylate (222 mg, 1.27 mmol) and potassium carbonate (183 mg, 1.27 mmol) were mixed in 5.5 ml of 1-methyl-2-pyrrolidinone. The reaction mixture was heated in microwave at 210° C. for 30 minutes. The mixture was applied to a SCX ion exchange cartridge (Varian bond-elute, 10 g), washed with methanol and then with a 2M ammonia in methanol solution. The combined basic fractions were concentrated in vacuo and the resulting residue purified by column chromatography eluting with a mixture of 2M ammonia in methanol and dichloromethane (0-2%) to afford the title product; MS (ES+) m/e 421 [M+H]⁺.

### Step 2: 5-[4-(3-cyclobutyl-2,3,4,5-tetrahydro-1H-3-benzazepin-7-yl)-1-piperidinyl]-2-pyrazinecarboxylic acid

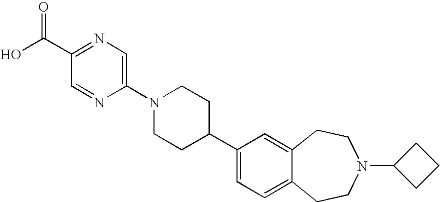

A solution of methyl 5-[4-(3-cyclobutyl-2,3,4,5-tetrahydro-1H-3-benzazepin-7-yl)-1-piperidinyl]-2-pyrazinecarboxylate (product of E226, step 1) (167 mg, 0.40 mmol) in dichloromethane (2.5 ml) was treated with conc. HCl (2.5 ml) and the resulting biphasic mixture heated in a microwave at 100° C. for 60 minutes. The mixture was concentrated in vacuo and azeotroped with toluene and dichloromethane to afford the crude product which was used directly in the next step.

### Step 3: 5-[4-(3-cyclobutyl-2,3,4,5-tetrahydro-1H-3-benzazepin-7-yl)-1-piperidinyl]-2-pyrazinecarbonyl chloride

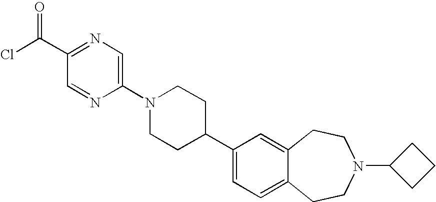

A suspension of 5-[4-(3-cyclobutyl-2,3,4,5-tetrahydro-1H-3-benzazepin-7-yl)-1-piperidinyl]-2-pyrazinecarboxylic acid (product of E226, step 2) (161 mg, 0.40 mmol) in dichloromethane (10 ml ) was treated with oxalyl chloride (0.15 ml, 1.75 mmol), followed by dimethylformamide (1 drop). The resulting clear solution was allowed to stir at room temperature for 1 hour. The mixture was then concentrated in vacuo and used directly in the next step.

### Step 4: 5-[4-(3-cyclobutyl -2,3,4,5-tetrahydro-1H-3-benzazepin-7-yl)-1-piperidinyl]-N-methyl-2-pyrazinecarboxamide

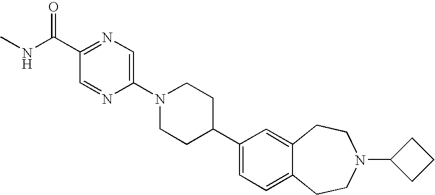

To a solution of 5-[4-(3-cyclobutyl-2,3,4,5-tetrahydro-1H-3-benzazepin-7-yl)-1-piperidinyl]-2-pyrazinecarbonyl chloride (product of E226, step 3) (169 mg, 0.397 mmol) in dichloromethane (10 ml) at 0° C. was added, dropwise, a 2M solution of methylamine in THF (4 ml, 7.94 mmol). The mixture was allowed to stir at 0° C. for 15 minutes and then at room temperature for 18 hours. The mixture was then concentrated in vacuo and purified by column chromatography eluting with a mixture of 2M ammonia in methanol and dichloromethane (2%). Recrystallisation from ethyl acetate afforded the title product; MS (ES+) m/e 420 [M+H]⁺.

### EXAMPLE 227

### 5-[4-(3-cyclobutyl-2,3,4,5-tetrahydro-1H-3-benzazepin-7-yl)-1-piperidinyl]-N-methyl-2-pyridinecarboxamide (E227)

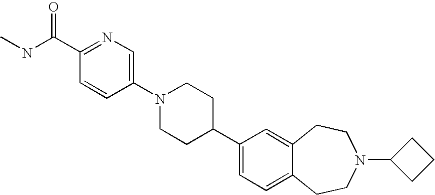

### Step 1: 1,1-dimethylethyl 5-[4-(3-cyclobutyl-2,3,4,5-tetrahydro-1H-3-benzazepin-7-yl)-1-piperidinyl]-2-pyridinecarboxylate

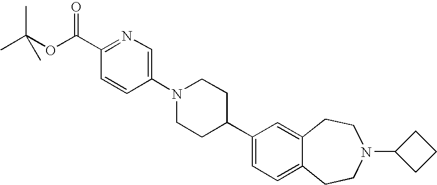

A mixture of 3-cyclobutyl-7-(4-piperidinyl)-2,3,4,5-tetrahydro-1H-3-benzazepine (285 mg, 1.0 mmol) (D12), 1,1-dimethylethyl 5-bromo-2-pyridinecarboxylate (351 mg, 1.3 mmol), tris (dibenzylidineacetone) dipalladium (23 mg, 0.02 mmol), caesium carbonate (482 mg, 1.5 mmol) and ‘xantphos’ (51 mg, 0.1 mmol) in 1,4-dioxan (8 ml) was heated at reflux for 18 hours. The mixture was filtered through Celite and evaporated. The residue was purified by column chromatography on silica eluting with 97-3 dichloromethane -2M ammonia in methanol to afford the title compound MS (AP+) m/e 462 [M+H]⁺.

### Step 2: 5-[4-(3-cyclobutyl-2,3,4,5-tetrahydro-1H-3-benzazepin-7-yl)-1-piperidinyl]-2-pyridinecarboxylic acid

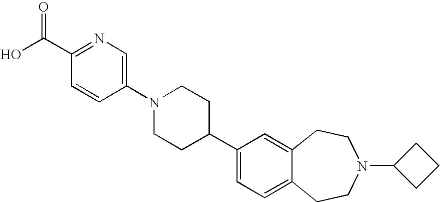

1,1-dimethylethyl 5-[4-(3-cyclobutyl-2,3,4,5-tetrahydro-1H-3-benzazepin-7-yl)-1-piperidinyl]-2-pyridinecarboxylate (product of E227, step 1) (650 mg, 1.4 mmol) was dissolved in trifluoroacetic acid (20 ml) and water (2 ml) and stirred at room temperature for 5 hours. The solvent was removed by evaporation in vacuo to obtain the title compound as a yellow oil which was used crude in the next step.

### Step 3: 5-[4-(3-cyclobutyl-2,3,4,5-tetrahydro-1H-3-benzazepin-7-yl)-1-piperidinyl]-N-methyl-2-pyridinecarboxamide

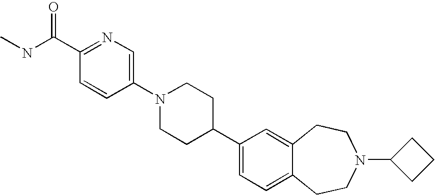

A mixture of 5-[4-(3-cyclobutyl-2,3,4,5-tetrahydro-1H-3-benzazepin-7-yl)-1-piperidinyl]-2-pyridinecarboxylic acid (product of E227, step 2) (320 mg, 0.79 mmol) and 1,1′-(oxomethanediyl)bis-1H-imidazole (400 mg, 2.5 mmol) in dry tetrahydrofuran (5 ml) was stirred at room temperature for 3 hours. A 2M solution of methylamine in tetrahydrofuran (10 ml, 20 mmol) was added and the mixture heated at 40° C. for 18 hours. This was poured into water and extracted with ethyl acetate. The extracts were dried (sodium sulphate) and evaporated. The residue was purified on an SCX ion exchange cartridge eluting with methanol and then 2M ammonia in methanol to afford the title compound as a cream powder MS (AP+) m/e 419 [M+H]⁺.

### EXAMPLE 228

### 1-{6-[4-(3-Cyclobutyl-2,3,4,5-tetrahydro-1H-3-benzazepin-7-yl)-1-piperidinyl]-3-pyridinyl}-2-pyrrolidinone (E228)

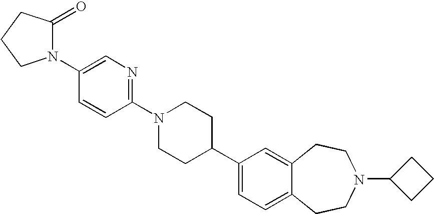

### Step 1: 3-Cyclobutyl-7-[1-(5-iodo-2-pyridinyl)-4-piperidinyl]-2,3,4,5-tetrahydro-1H-3-benzazepine

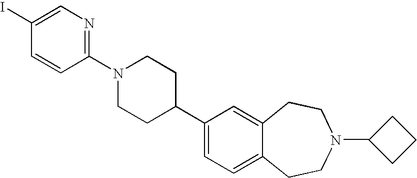

A mixture of 3-cyclobutyl-7-(4-piperidinyl)-2,3,4,5-tetrahydro-1H-3-benzazepine (D12) (79 mg, 0.28 mmol), 2-chloro-5-iodopyridine (107 mg, 0.44 mmol) and potassium carbonate

(119 mg, 0.87 mmol) in N-methylpyrrolidinone (4 ml) was heated in a microwave reactor at 100° C. (high absorbance) for 30 minutes and then at 180° C. for 60 minutes. The mixture was purified on an SCX ion exchange cartridge eluting with methanol and then 2M ammonia in methanol. The basic fractions were combined and evaporated. The residue was purified by column chromatography on silica eluting with 98-2 dichloromethane—2M ammonia in methanol to afford the title compound MS (AP+) m/e 488 [M+H]⁺.

### Step 2: 1-{6-[4-(3-Cyclobutyl-2,3,4,5-tetrahydro-1H-3-benzazepin-7-yl)-1-piperidinyl]-3-pyridinyl}-2-pyrrolidinone

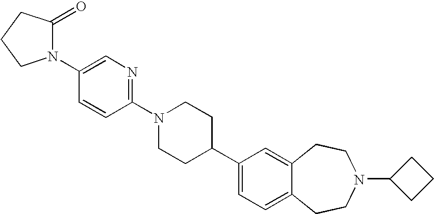

A mixture of 3-cyclobutyl-7-[1-(5-iodo-2-pyridinyl)-4-piperidinyl]-2,3,4,5-tetrahydro-1H-3-benzazepine (product of E228, step 1) (75 mg, 0.15 mmol), 2-pyrrolidinone (0.04 ml, 0.52 mmol), potassium carbonate (79 mg, 0.55 mmol), copper (I) iodide (18 mg, 0.09 mmol) and N,N′-dimethyl-1,2-ethanediamine (0.02 ml, 0.18 mmol) in 1,4-dioxan (3 ml) was heated in a microwave reactor at 140° C. (high absorbance) for 20 minutes. The mixture was purified on an SCX ion exchange cartridge eluting with methanol and then 2M ammonia in methanol. The basic fractions were combined and evaporated. The residue was purified by column chromatography on silica eluting with 97-3 dichloromethane -2M ammonia in methanol to afford the title compound MS (AP+) m/e 445 [M+H]⁺.

### EXAMPLE 229

### 3-Cyclobutyl-7-(4-pyridinyl)-2,3,4,5-tetrahydro-1H-3-benzazepine (E229)

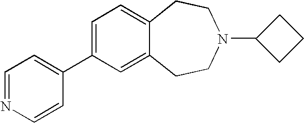

### Step 1: 1,1-Dimethylethyl 7-(4-pyridinyl)-1,2,4,5-tetrahydro-3H-3-benzazepine-3-carboxylate

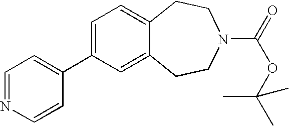

Tetrakis triphenylphosphino palladium (0) (375 mg, 0.33 mmol) was added to a mixture of 1,1-dimethylethyl 7-{[(trifluoromethyl)sulfonyl]oxy}-1,2,4,5-tetrahydro-3H-3-benzazepine-3-carboxylate (D1) (1.29 g, 3.25 mmol) and 4-pyridinylboronic acid (0.6 g, 5.0 mmol) in dimethoxyethane (40 ml) and 1 M sodium carbonate solution (4 ml). The resulting mixture was heated at reflux for 3 hours and allowed to cool to room temperature. The mixture was evaporated in vacuo and the residue purified by silica column chromatography eluting with 1-1 pentane-ethyl acetate to afford the title compound as a colourless crystalline solid (0.62 g, 59%) MS (AP+) m/e 325 [M+H]⁺.

### Step 2: 7-(4-Pyridinyl)-2,3,4,5-tetrahydro-1H-3-benzazepine

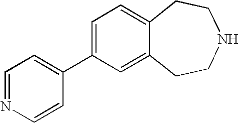

A solution of 1,1-dimethylethyl 7-(4-pyridinyl)-1,2,4,5-tetrahydro-3H-3-benzazepine-3-carboxylate (product of E229, step 1) (1.15 g, 3.54 mmol) in dichloromethane (10 ml) was added drop-wise to a 4M solution of hydrogen chloride in dioxan (10 ml). The resulting mixture was stirred at room temperature for 1 hour and was diluted with ethyl acetate. The resulting solid was collected by filtration and dissolved in water. The pH was adjusted to 12 by the addition of 2M sodium hydroxide solution and the mixture extracted with ethyl acetate. The extracts were combined, dried (sodium sulphate) and evaporated to give a colourless powder (0.52 g, 66%) MS (AP+) m/e 225 [M+H]⁺.

### Step 3: 3-Cyclobutyl-7-(4-pyridinyl)-2,3,4,5-tetrahydro-1H-3-benzazepine

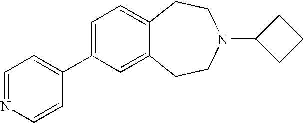

Sodium triacetoxyborohydride (0.38 g, 1.8 mmol) was added to a mixture of 7-(4-pyridinyl)-2,3,4,5-tetrahydro-1H-3-benzazepine (product of E229, step 2) (0.2 g, 0.9 mmol) and cyclobutanone (0.33 ml, 1.8 mmol) in dichloromethane (3 ml) and the mixture stirred for 3 days. The mixture was diluted with methanol and purified on an SCX ion exchange cartridge eluting with methanol and then 2M ammonia in methanol. The basic fractions were combined and evaporated, the residue was purified by column chromatography on silica eluting with 97-3 dichloromethane -2M ammonia in methanol to afford the title compound as a colourless crystalline solid (0.2 g, 80%) MS (AP+) m/e 279 [M+H]⁺.

### EXAMPLE 230

### 6-[4-(3-cyclopentyl-2,3,4,5-tetrahydro-1H-3-benzazepin-7-yl)-1-piperidinyl]-N-methyl-3-pyridinecarboxamide (E230)

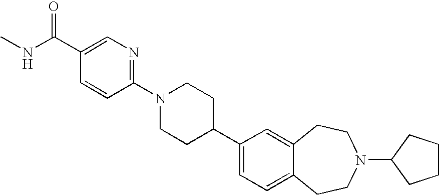

### Step 1: 3-cyclopentyl-7-(4-pyridinyl)-2,3,4,5-tetrahydro-1H-3-benzazepine

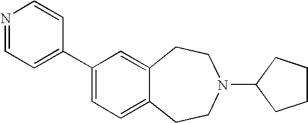

A mixture of 7-(4-pyridinyl)-2,3,4,5-tetrahydro-1H-3-benzazepine (3.74 g, 16.7 mmol) (product of E229, step 2), cyclopentanone (2.95 ml, 33.3 mmol), molecular sieves (4 Å, 800 mg) and a 5% acetic acid/dichloromethane solution (120 ml) was stirred at room temperature for 30 minutes. Sodium triacetoxyborohydride (7.0 g, 33.3 mmol) was then added and the mixture stirred at room temperature for 18 hours. The mixture was diluted with methanol and applied to a SCX ion exchange cartridge (Varian bond-elute, 10 g), washed with methanol and then with a 2M ammonia in methanol solution. The combined basic fractions were concentrated in vacuo to afford the title product; MS (ES+) m/e 293 [M+H]⁺.

### Step 2: 3-cyclopentyl-7-(4-piperidinyl)-2,3,4,5-tetrahydro-1H-3-benzazepine

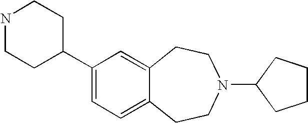

3-cyclopentyl-7-(4-pyridinyl)-2,3,4,5-tetrahydro-1H-3-benzazepine (product of E230, step 1) (4.8 g, 16.4 mmol) was dissolved in a mixture of ethanol:acetic acid (10:1) (198 ml). Platinum oxide (480 mg) was added and the reaction mixture was heated at 50° C. under hydrogen (50 psi) for 24 hours. The mixture was then concentrated in vacuo and applied to a SCX ion exchange cartridge (Varian bond-elute, 10 g), washed with methanol and then with a 2M ammonia in methanol solution. The combined basic fractions were concentrated in vacuo and purified by column chromatography eluting with a mixture of 2M ammonia in methanol and dichloromethane (5-10%) to afford the title product; MS (ES+) m/e 299 [M+H]⁺.

### Step 3: 6-[4-(3-cyclopentyl-2,3,4,5-tetrahydro-1H-3-benzazepin-7-yl)-1-piperidinyl]-N-methyl-3-pyridinecarboxamide

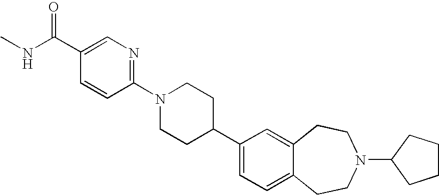

3-cyclopentyl-7-(4-piperidinyl)-2,3,4,5-tetrahydro-1H-3-benzazepine (product of E230, step 2) (112 mg, 0.38 mmol), 6-chloro-N-methyl-3-pyridinecarboxamide (PCT Int. Appl. (2002), WO 2002046186)(84 mg, 0.49 mmol), and potassium carbonate (116 mg, 0.83 mmol) were mixed in 2 ml of 1-methyl-2-pyrrolidinone. The reaction mixture was heated in microwave at 210° C. for 30 minutes. The mixture was applied to a SCX ion exchange cartridge (Varian bond-elute, 10 g), washed with methanol and then with a 2M ammonia in methanol solution. The combined basic fractions were concentrated in vacuo and the resulting residue purified by column chromatography eluting with a mixture of 2M ammonia in methanol and dichloromethane (1-3%) to afford the title product; MS (ES+) m/e 433 [M+H]⁺.

### EXAMPLE 231

### 3-Cyclopentyl-7-{1-[6-(trifluoromethyl)-3-pyridinyl]-4-piperidinyl}-2,3,4,5-tetrahydro-1H-3-benzazepine (E231)

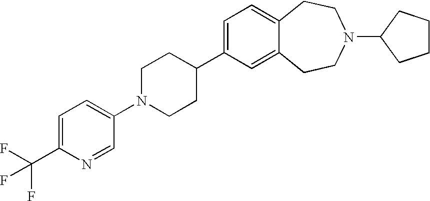

3-Cyclopentyl-7-(4-piperidinyl)-2,3,4,5-tetrahydro-1H-3-benzazepine (product of E230, step 2) (0.1 g, 0.33 mmol), 2-trifluoromethyl-5-bromo pyridine (83 mg, 0.37 mmol), tris (dibenzylidineacetone) dipalladium (13 mg, 0.02 mmol 2′-(dicyclohexylphosphanyl)-N,N-dimethyl-2-biphenylamine (20 mg, 0.06 mmol) and sodium tert-butoxide (63 mg, 0.66 mmol) in dioxan (3 ml) was heated in a microwave reactor at 120° C. for 5 minutes. The crude mixture was purified on an SCX ion exchange cartridge eluting with methanol and then 2M ammonia in methanol. The basic fractions evaporated and the residue purified by column chromatography on silica eluting with 95-5 dichloromethane—2M ammonia in methanol to afford the title compound as a yellow solid (46 mg, 31%) MS (AP+) m/e 444 [M+H]⁺.

### EXAMPLE 232

### Methyl 5-[4-(3-cyclopentyl-2,3,4,5-tetrahydro-1H-3-benzazepin-7-yl)-1-piperidinyl]-2-pyrazinecarboxylate (E232)

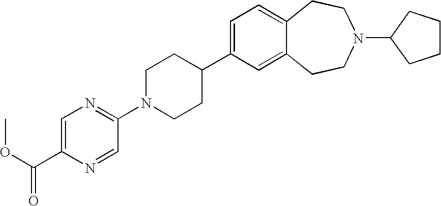

A mixture of 3-Cyclopentyl-7-(4-piperidinyl)-2,3,4,5-tetrahydro-1H-3-benzazepine (product of E230, step 2) (0.5 g, 1.68 mmol), potassium carbonate (0.46 g, 3.35 mmol) and methyl 5-chloro-2-pyrazinecarboxylate (0.58 g, 3.35 mmol) in dimethylformamide (20 ml) was heated at 90° C. for 3 hours. The crude mixture was purified on an SCX ion exchange cartridge eluting with methanol and then 2M ammonia in methanol. The basic fractions evaporated and the residue purified by column chromatography on silica eluting with 95-5 dichloromethane -2M ammonia in methanol to afford the title compound as a yellow solid (0.55 g, 75%) MS (AP+) m/e 435 [M+H]⁺.

### EXAMPLE 233

### 5-[4-(3-Cyclopentyl-2,3,4,5-tetrahydro-1H-3-benzazepin-7-yl)-1-piperidinyl]-N-methyl-2-pyrazinecarboxamide (E233)

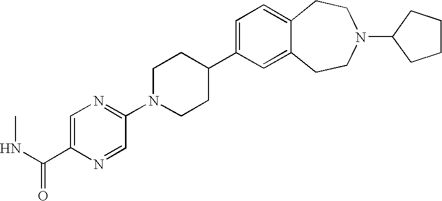

### Step 1: 5-[4-(3-Cyclopentyl-2,3,4,5-tetrahydro-1H-3-benzazepin-7-yl)-1-piperidinyl]-2-pyrazinecarboxylic acid

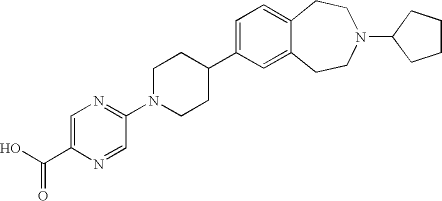

A solution of methyl 5-[4-(3-cyclopentyl-2,3,4,5-tetrahydro-1H-3-benzazepin-7-yl)-1-piperidinyl]-2-pyrazinecarboxylate (E232) (100 mg, 0.23 mmol) in methanol (5 ml) was treated with 1 M sodium hydroxide solution (1 ml) and heated at reflux for 90 minutes. The mixture was acidified using 2 M hydrochloric acid and purified on an SCX ion exchange column eluting with methanol and then 2M ammonia in methanol. The basic fractions were evaporated to afford the title compound as a yellow solid MS (AP+) m/e 421 [M+H]⁺.

### Step 2: 5-[4-(3-Cyclopentyl-2,3,4,5-tetrahydro-1H-3-benzazepin-7-yl)-1-piperidinyl]-N-methyl-2-pyrazinecarboxamide

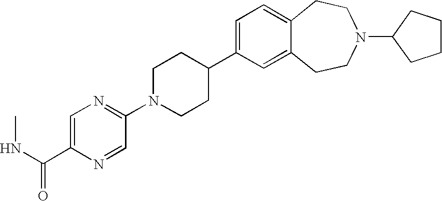

A solution of 5-[4-(3-Cyclopentyl-2,3,4,5-tetrahydro-1H-3-benzazepin-7-yl)-1-piperidinyl]-2-pyrazinecarboxylic acid (product of E233, step 1) (100 mg, 0.24 mmol) in tetrahydrofuran (3 ml) was treated with N,N′-dicyclohexylcarbodiimide (39 mg, 0.24 mmol) and stirred at room temperature for 18 hours. This mixture was treated with 2M methylamine in tetrahydrofuran (0.24 ml, 0.48 mmol) and stirred at room temperature for 18 hours. The mixture was purified on an SCX ion exchange column eluting with methanol and then 2M ammonia in methanol.

The basic fractions were evaporated to afford the title compound as a colourless solid MS (AP+) m/e 434 [M+H]⁺.

### EXAMPLE E234

### 4-({4-[3-(2-methylcyclopentyl)-2,3,4,5-tetrahydro-1H-3-benzazepin-7-yl]-1-piperidinyl}carbonyl)benzonitrile (E234)

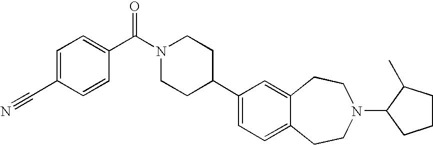

### Step 1: 1,1-dimethylethyl 7-(4-piperidinyl)-1,2,4,5-tetrahydro-3H-3-benzazepine-3-carboxylate

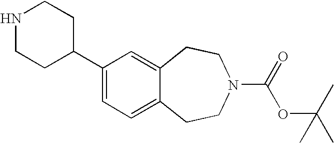

1,1-Dimethylethyl 7-(1-{[(phenylmethyl)oxy]carbonyl}-1,2,3,6-tetra hydro-4-pyridinyl)-1,2,4,5-tetrahydro-3H-3-benzazepine-3-carboxylate (D9) (2.09 g, 4.52 mmol) was dissolved ethanol (140 ml). Palladium on charcoal (720 mg, 10% palladium/charcoal paste) was added and the reaction mixture was heated at 40° C. under hydrogen (50 psi) for 24 hours. The mixture was then filtered through celite and concentrated in vacuo. The resulting oil was re-dissolved in dichloromethane and evaporated three times and then re-dissolved in diethyl ether and evaporated twice to yield the title product; MS (ES+) m/e 331 [M+H]⁺.

### Step 2: 1,1-dimethylethyl 7-{1-[(4-cyanophenyl)carbonyl]-4-piperidinyl}-1,2,4,5-tetrahydro-3H-3-benzazepine-3-carboxylate

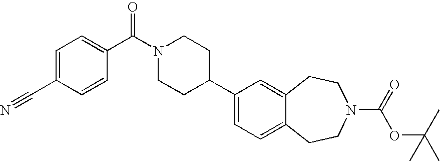

A mixture of 4-cyanobenzoic acid (1.39 g, 4.66 mmol), N-Cyclohexylcarbodiimide N′-methyl polystyrene (4.48 g, 9.32 mmol), and 1-hydroxybenzotriazole (1.25 g, 9.32 mmol) in dry dimethylformamide (20 ml) were stirred under argon at room temperature for 60 minutes. A solution of 1,1-dimethylethyl 7-(4-piperidinyl)-1,2,4,5-tetrahydro-3H-3-benzazepine-3-carboxylate (product of E234, step 1)(1.54 g, 4.66 mmol) in dry dimethylformamide (10 ml) was added, and the reaction mixture left to stir at room temperature for 3 hours. The mixture was filtered, diluted with water (60 ml) and extracted with ethyl acetate (4×60 ml). The combined organic phases were washed with a saturated sodium bicarbonate solution (2×60 ml) and brine (2×60 ml). The organic phase was dried over magnesium sulphate and concentrated in vacuo. The resulting residue was purified by column chromatography eluting with a mixture of ethyl acetate in pentane (20-50%) to afford the title product; MS (ES+) m/e 360 [M-BOC]⁺.

### Step 3: 4-{[4-(2,3,4,5-tetrahydro-1H-3-benzazepin-7-yl)-1-piperidinyl]carbonyl}benzonitrile

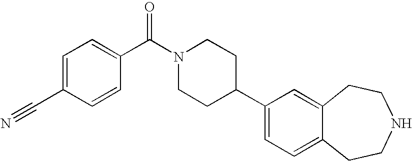

1,1-dimethylethyl 7-{1-[(4-cyanophenyl)carbonyl]-4-piperidinyl}-1,2,4,5-tetrahydro-3H-3-benzazepine-3-carboxylate (product of E234, step 2) (1.61 g, 3.51 mmol) was dissolved in dichloromethane (5 ml) at 0° C. and treated with trifluoroacetic acid (5 ml). The solution was stirred at room temperature for 5 hours and concentrated in vacuo, co-evaporating with dichloromethane. The residue was re-dissolved in dichloromethane and ammonia 0.88 (10 ml) was added. The organic phase was separated and the aqueous phase extracted with dichloromethane (4×10 ml). The combined organic phases were washed with water (10 ml), dried over magnesium sulphate and concentrated in vacuo to yield a yellow oil which was co-evaporated with diethyl ether to afford the title compound. MS (ES+) m/e 360 [M+H]⁺.

### Step 4: 4-({4-[3-(2-methylcyclopentyl)-2,3,4,5-tetrahydro-1H-3-benzazepin-7-yl]-1-piperidinyl}carbonyl)benzonitrile

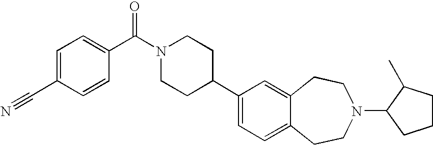

4-{[4-(2,3,4,5-tetrahydro-1H-3-benzazepin-7-yl)-1-piperidinyl]carbonyl}benzonitrile (product of E234, step 3)(160 mg, 0.45 mmol) was dissolved in anhydrous dichloromethane (3 ml) and treated with 2-methylcyclopentanone (0.1 ml, 0.93 mmol), followed by acetic acid (0.5 ml). The reaction mixture was allowed to stir at room temperature for one hour. Sodium triacetoxyborohydride (250 mg, 1.32 mmol) was added and the reaction mixture stirred at room temperature for 18 hours. The mixture was diluted with methanol and applied to a SCX cartridge (Varian bond-elute, 5 g) and washed with methanol and then a mixture of 2M ammonia/methanol. The basic fractions were combined and solvent was removed in vacuo. The resulting residue was purified by column chromatography eluting with a mixture of methanol and dichloromethane (0-10%) to afford the title product; MS (ES+) m/e 442 [M+H]⁺.

### EXAMPLE 235-236 (E235-E236)

Examples 235-236 were prepared using an analogous method to that described for Example 234 step 4 from 4-{[4-(2,3,4,5-tetrahydro-1H-3-benzazepin-7-yl)-1-piperidinyl]carbonyl}benzonitrile (product of E234, step 3) and the appropriate ketone indicated in the table below.

| | | LC/MS |
|---|---|---|
| Example | Ketone | (M + H⁺⁾ |
| 4-{[4-(3-cyclohexyl-2,3,4,5-tetrahydro-1H-3- | cyclohexanone | 441 |
| benzazepin-7-yl)-1- | | |
| piperidinyl]carbonyl}benzonitrile(E235) | | |
| 4-{[4-(3-cyclopentyl-2,3,4,5-tetrahydro- | cyclopentanone | 428 |
| 1H-3-benzazepin-7-yl)-1- | | |
| piperidinyl]carbonyl}benzonitrile (E236) | | |

### EXAMPLE 237

### N-(4-cyanophenyl)-4-(3-cyclobutyl-2,3,4,5-tetrahydro-1H-3-benzazepin-7-yl)-1-piperidinecarboxamide (E237)

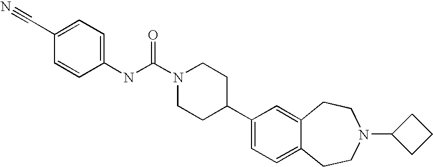

4-Aminobenzonitrile (0.059 g, 0.50 mmol) in dry dichloromethane (5 ml) was added dropwise to a 20% solution of phosgene in toluene (0.36 ml, 0.75 mmol) and the mixture was stirred at room temperature for 30 minutes. The solvents were removed in vacuo and the residue dissolved in dichloromethane (5 ml) and treated with diisopropylethlamine (0.12 ml, 0.25 mmol) followed by 3-cyclobutyl-7-(4-piperidinyl)-2,3,4,5-tetrahydro-1H-3-benzazepine (D12) (0.07 g, 0.25 mmol) in dichloromethane (1 ml). The mixture was stirred at room temperature for 2 hours after which it was diluted with methanol and applied to an SCX cartridge (Varian bond-elute) and washed with methanol and then a mixture of 2M ammonia/methanol. The basic fractions were combined and the solvent removed in vacuo. The residue was purified by column chromatography eluting with a gradient of dichloromethane to 6% (2M 0.880 ammonia/methanol)/dichloromethane to afford the title compound; MS (ES+), m/e 429 [M+H]⁺

### EXAMPLE 238

### N-(4-Cyanophenyl)-4-(3-cyclobutyl-2,3,4,5-tetrahydro-1H-3-benzazepin-7-yl)-1-piperidinecarbothioamide (E238)

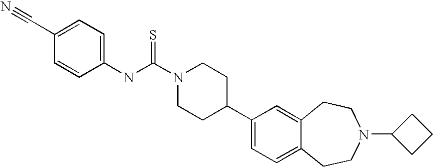

3-Cyclobutyl-7-(4-piperidinyl)-2,3,4,5-tetrahydro-1H-3-benzazepine (D12) (0.15 g, 0.52 mmol) and 4-isothiocyanatobenzonitrile (0.13 g, 0.8 mmol) in dry dimethylformamide (10 ml) were stirred at room temperature for 3 hours. The reaction mixture was diluted with methanol and applied to an SCX cartridge (Varian bond-elute) and washed with methanol and then a mixture of 2M ammonia/methanol. The basic fractions were combined and the solvent removed in vacuo to afford the title compound; MS (ES+), m/e 445 [M+H]⁺

### EXAMPLE 239

### 3-cyclobutyl-7-[4-(1-naphthalenylcarbonyl)-1-piperazinyl]-2,3,4,5-tetrahydro-1H-3-benzazepine (E239)

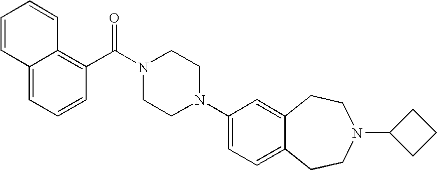

3-Cyclobutyl-7-(1-piperazinyl)-2,3,4,5-tetrahydro-1H-3-benzazepine (D7) (10 mg, 0.04 mmol) was dissolved in dichloromethane (1 ml) and treated with 1-naphthalenecarboxylic acid (7 mg, 0.04 mmol) and diisopropylethylamine (7 μl, 0.04 mmol). The reaction mixture was then passed through an aminopropyl cartridge and purified using reverse phase chromatography to yield the title product, MS (ES+) m/e 440 [M+H]⁺.

### EXAMPLE 240

### 4-(3-cyclobutyl-2,3,4,5-tetrahydro-1H-3-benzazepin-7-yl)-N-(4-fluoro phenyl)-1-piperazinecarboxamide (E240)

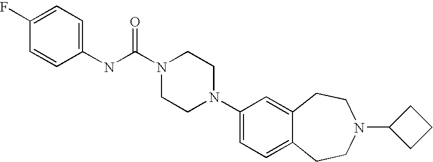

Diisopropylethylamine (0.15 ml, 0.86 mmol) in dry THF (3 ml) was cooled to 0° C. and treated with triphosgene (0.052 g, 0.18 mmol). The mixture was stirred for 5 minutes and treated dropwise with a solution of 3-cyclobutyl-7-(1-piperazinyl)-2,3,4,5-tetrahydro-1H-3-benzazepine (D7) (0.10 g, 0.36 mmole) and diisopropylethylamine (0.15 ml, 0.86 mmol). After stirring for 20 minutes, 4-fluoroaniline (0.034 ml, 0.35 mmol) was added dropwise and the mixture was stirred overnight at room temperature. Ethyl acetate was added to the reaction mixture, which was subsequently filtered through celite. The filtrate was washed with water and brine, dried (MgSO₄) and concentrated in vacuo. The residue was dissolved in methanol and applied to an SCX cartridge (Varian bond-elute) and washed with methanol and then a mixture of 2M ammonia/methanol. The basic fractions were combined and the solvent removed in vacuo. The residue was triturated with dichloromethane and the resulting solid filtered and dried to afford the title compound; MS (ES+), m/e 423 [M+H]⁺

### EXAMPLES 241 TO 244

Examples 241 to 244 were prepared using an analogous method to that described for Example 240 from 3-cyclobutyl-7-(4-piperidinyl)-2,3,4,5-tetrahydro-1H-3-benzazepine (D12) and the appropriate amine as indicated in the table.

| | | LC/MS |
|---|---|---|
| Example | Amine | (M + H)⁺ |
| 4-(3-Cyclobutyl-2,3,4,5-tetrahydro-1H-3-benzazepin-7- | 4-Methoxyaniline | 434 |
| yl)-N-[4-(methyloxy)phenyl]-1-piperidinecarboxamide | | |
| (E241) | | |
| N-(3-Cyanophenyl)-4-(3-cyclobutyl-2,3,4,5-tetrahydro- | 3-Aminobenzonitrile | 429 |
| 1H-3-benzazepin-7-yl)-1-piperidinecarboxamide | | |
| (E242) | | |
| 3-Cyclobutyl-7-[1-(1,3-dihydro-2H-isoindol-2- | Isoindoline | 430 |
| ylcarbonyl)-4-piperidinyl]-2,3,4,5-tetrahydro-1H-3- | | |
| benzazepine (E243) | | |
| N-(6-cyano-3-pyridinyl)-4-(3-cyclobutyl-2,3,4,5- | 5-Amino-2- | 430 |
| tetrahydro-1H-3-benzazepin-7-yl)-1- | cyanopyridine | |
| piperidinecarboxamide (E244) | | |

### EXAMPLE 245

### 4-(3-cyclobutyl-2,3,4,5-tetrahydro-1H-3-benzazepin-7-yl)-N-3-pyridinylbenzamide (E245)

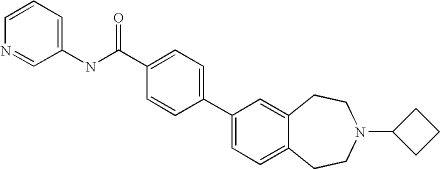

### Step 1: 1,1-Dimethylethyl 7-{4-[(3-pyridinylamino)carbonyl]phenyl}-1,2,4,5-tetrahydro-3H-3-benzazepine-3-carboxylate

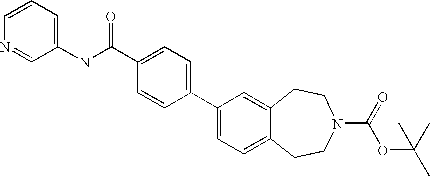

4-Iodo-N-3-pyridinylbenzamide (D13) (0.086 g, 0.32 mmole) in dimethoxyethane (7 ml) and 2M aqueous sodium carbonate solution (0.23 ml) was treated with 1,1-dimethylethyl 7-(4,4,5,5-tetramethyl-1,3,2-dioxaborolan-2-yl)-1,2,4,5-tetrahydro-3H-3-benzazepine-3-carboxylate (E194 Step 1) (0.10 g, 0.27 mmol) and tetrakis(triphenyl phosphinepalladium(0)) (0.009 g, 0.008 mmol) and heated at 80° C. for 18 hours. The reaction mixture was partitioned between ethyl acetate and water and the ethyl acetate layer dried (MgSO₄) and concentrated in vacuo. The residue was purified by column chromatography eluting with a gradient of dichloromethane to 10% (2M 0.880 ammonia/methanol)/dichloromethane to afford the title compound; MS (ES+), m/e 344 [M-BOC+H]⁺

### Step 2: N-3-Pyridinyl-4-(2,3,4,5-tetrahydro-1H-3-benzazepin-7-yl)benzamide

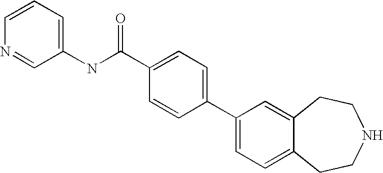

1,1-Dimethylethyl 7-{4-[(3-pyridinylamino)carbonyl]phenyl}-1,2,4,5-tetrahydro-3H-3-benzazepine-3-carboxylate (product of E245, step 1) (0.059 g, 0.13 mmol) in dichloromethane (5 ml) was cooled to 0° C. and treated dropwise with trifluoroacetic acid (1 ml). The mixture was stirred at room temperature for 1 hour after which the solvent was removed in vacuo and the residue dissolved in methanol and applied to an SCX cartridge (Varian bond-elute) and washed with methanol and then a mixture of 2M ammonia/methanol. The basic fractions were combined and the solvent removed in vacuo. The residue was purified by column chromatography eluting with a gradient of dichloromethane to 5% (2M 0.880 ammonia/methanol)/dichloromethane to afford the title compound; MS (ES+), m/e 344 [M+H]⁺

### Step 3: 4-(3-Cyclobutyl-2,3,4,5-tetrahydro-1H-3-benzazepin-7-yl)-N-3-pyridinylbenzamide

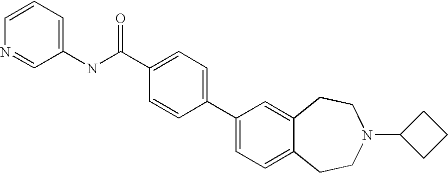

N-3-Pyridinyl-4-(2,3,4,5-tetrahydro-1H-3-benzazepin-7-yl)benzamide (product of E245, step 2) (0.031 g, 0.090 mmol) in dichloromethane (3 ml) and glacial acetic acid (0.15 ml) was treated with cyclobutanone (0.02 ml, 0.18 mmol), and 4 Å molecular sieves (50 mg) and stirred at room temperature for 10 minutes. Sodium triacetoxyborohydride (0.038 g, 0.18 mmol) was added and the mixture stirred for 18 hours at room temperature. The reaction mixture was diluted with methanol and applied to an SCX cartridge (Varian bond-elute) and washed with methanol and then a mixture of 2M ammonia/methanol. The basic fractions were combined and the solvent removed in vacuo to afford the title compound; MS (ES+), m/e 398 [M+H]⁺

### EXAMPLE 246

### Phenylmethyl 4-(3-cyclobutyl-2,3,4,5-tetrahydro-1H-3-benzazepin-7-yl)-1-piperazinecarboxylate (E246)

The preparation of E246 is described in Description 4.

### EXAMPLE 247

### Phenylmethyl 4-(3-cyclopentyl-2,3,4,5-tetrahydro-1H-3-benzazepin-7-yl)-1-piperazinecarboxylate (E247)

The preparation of E247 is described in Description 5.

### EXAMPLE 248

### 3-Cyclopentyl-7-(1-piperazinyl)-2,3,4,5-tetrahydro-1H-3-benzazepine (E248)

The preparation of E248 is described in Description 6.

### EXAMPLE 249

### 3-Cyclobutyl-7-(1-piperazinyl)-2,3,4,5-tetrahydro-1H-3-benzazepine (E249)

The preparation of E249 is described in Description 7.

### EXAMPLE 250

### Phenylmethyl 4-(3-cyclobutyl-2,3,4,5-tetrahydro-1H-3-benzazepin-7-yl)-3,6-dihydro-1(2H)-pyridinecarboxylate (E250)

The preparation of E250 is described in Description 11.

### EXAMPLE 251

### 3-Cyclobutyl-7-(4-piperidinyl)-2,3,4,5-tetrahydro-1H-3-benzazepine (E251)

The preparation of E251 is described in Description 12.

### EXAMPLE 252

### Methyl 6-(3-cyclobutyl-2,3,4,5-tetrahydro-1H-3-benzazepin-7-yl)-3-pyridinecarboxylate (E252)

The preparation of E252 is described in Step 3 of Example 198.

### EXAMPLE 253

### 6-(3-Cyclobutyl-2,3,4,5-tetrahydro-1H-3-benzazepin-7-yl)-3-pyridinecarboxylic acid (E253)

The preparation of E253 is described in Step 4 of Example 198.

### EXAMPLE 254

### methyl 5-[4-(3-cyclobutyl-2,3,4,5-tetrahydro-1H-3-benzazepin-7-yl)-1-piperidinyl]-2-pyrazinecarboxylate (E254)

The preparation of E254 is described in Step 1 of Example 226.

### EXAMPLE 255

### 5-[4-(3-cyclobutyl-2,3,4,5-tetrahydro-1H-3-benzazepin-7-yl)-1-piperidinyl]-2-pyrazinecarboxylic acid (E255)

The preparation of E255 is described in Step 2 of Example 226.

### EXAMPLE 256

### 1,1-dimethylethyl 5-[4-(3-cyclobutyl-2,3,4,5-tetrahydro-1H-3-benzazepin-7-yl)-1-piperidinyl]-2-pyridinecarboxylate (E256)

The preparation of E256 is described in Step 1 of Example 227.

### EXAMPLE 257

### 5-[4-(3-cyclobutyl-2,3,4,5-tetrahydro-1H-3-benzazepin-7-yl)-1-piperidinyl]-2-pyridinecarboxylic acid (E257)

The preparation of E257 is described in Step 2 of Example 227.

### EXAMPLE 258

### 3-Cyclobutyl-7-[1-(5-iodo-2-pyridinyl)-4-piperidinyl]-2,3,4,5-tetrahydro-1H-3-benzazepine (E258)

The preparation of E258 is described in Step 1 of Example 228.

### EXAMPLE 259

### 3-cyclopentyl-7-(4-pyridinyl)-2,3,4,5-tetrahydro-1H-3-benzazepine (E259)

The preparation of E259 is described in Step 1 of Example 230.

### EXAMPLE 260

### 3-cyclopentyl-7-(4-piperidinyl)-2,3,4,5-tetrahydro-1H-3-benzazepine (E260)

The preparation of E260 is described in Step 2 of Example 230.

### EXAMPLE 261

### 5-[4-(3-Cyclopentyl-2,3,4,5-tetrahydro-1H-3-benzazepin-7-yl)-1-piperidinyl]-2-pyrazinecarboxylic acid (E261)

The preparation of E261 is described in Step 1 of Example 233.

### EXAMPLE 262

### (5R)-3-(3-Cyclobutyl-2,3,4,5-tetrahydro-1H.3-benzazepin-7-yl)-5-(hydroxymethyl)-1,3-oxazolidin-2-one (E262)

The preparation of E262 is described in Step 4 of Example 193.

Abbreviations

SCX: Strong cation exchange

Biological Data

A membrane preparation containing histamine H3 receptors may be prepared in accordance with the following procedures:

(i) Generation of Histamine H3 Cell Line

DNA encoding the human histamine H3 gene (Huvar, A. et al. (1999) Mol. Pharmacol. 55(6), 1101-1107) was cloned into a holding vector, pcDNA3.1 TOPO (InVitrogen) and its cDNA was isolated from this vector by restriction digestion of plasmid DNA with the enzymes BamH1 and Not-1 and ligated into the inducible expression vector pGene (InVitrogen) digested with the same enzymes. The GeneSwitch™ system (a system where in transgene expression is switched off in the absence of an inducer and switched on in the presence of an inducer) was performed as described in U.S. Pat. Nos. 5,364,791; 5,874,534; and 5,935,934. Ligated DNA was transformed into competent DH5α E. coli host bacterial cells and plated onto Luria Broth (LB) agar containing Zeocin™ (an antibiotic which allows the selection of cells expressing the sh ble gene which is present on pGene and pSwitch) at 50 μg ml⁻¹. Colonies containing the re-ligated plasmid were identified by restriction analysis. DNA for transfection into mammalian cells was prepared from 250 ml cultures of the host bacterium containing the pGeneH3 plasmid and isolated using a DNA preparation kit (Qiagen Midi-Prep) as per manufacturers guidelines (Qiagen). CHO K1 cells previously transfected with the pSwitch regulatory plasmid (InVitrogen) were seeded at 2×10e6 cells per T75 flask in Complete Medium, containing Hams F12 (GIBCOBRL, Life Technologies) medium supplemented with 10% v/v dialysed foetal bovine serum, L-glutamine, and hygromycin (100 μg ml⁻¹), 24 hours prior to use. Plasmid DNA was transfected into the cells using Lipofectamine plus according to the manufacturers guidelines (InVitrogen). 48 hours post transfection cells were placed into complete medium supplemented with 500 μg ml⁻¹ Zeocin™.

10-14 days post selection 10 nM Mifepristone (InVitrogen), was added to the culture medium to induce the expression of the receptor. 18 hours post induction cells were detached from the flask using ethylenediamine tetra-acetic acid (EDTA; 1:5000; InVitrogen), following several washes with phosphate buffered saline pH 7.4 and resuspended in Sorting Medium containing Minimum Essential Medium (MEM), without phenol red, and supplemented with Earles salts and 3% Foetal Clone II (Hyclone). Approximately 1×10e7 cells were examined for receptor expression by staining with a rabbit polyclonal antibody, 4a, raised against the N-terminal domain of the histamine H3 receptor, incubated on ice for 60 minutes, followed by two washes in sorting medium. Receptor bound antibody was detected by incubation of the cells for 60 minutes on ice with a goat anti rabbit antibody, conjugated with Alexa 488 fluorescence marker (Molecular Probes). Following two further washes with Sorting Medium, cells were filtered through a 50 μm Filcon™ (BD Biosciences) and then analysed on a FACS Vantage SE Flow Cytometer fitted with an Automatic Cell Deposition Unit. Control cells were non-induced cells treated in a similar manner. Positively stained cells were sorted as single cells into 96-well plates, containing Complete Medium containing 500 μg ml⁻¹ Zeocin™ and allowed to expand before reanalysis for receptor expression via antibody and ligand binding studies. One clone, 3H3, was selected for membrane preparation.

(ii) Membrane Preparation from Cultured Cells

All steps of the protocol are carried out at 4° C. and with pre-cooled reagents. The cell pellet is resuspended in 10 volumes of homogenisation buffer (50 mM N-2-hydroxyethylpiperazine-N′-2-ethanesulfonic acid (HEPES), 1 mM ethylenediamine tetra-acetic acid (EDTA), pH 7.4 with KOH, supplemented with 10e-6M leupeptin (acetyl-leucyl-leucyl-arginal; Sigma L2884), 25 μg/ml bacitracin (Sigma B0125), 1 mM phenylmethylsulfonyl fluoride (PMSF) and 2×10e-6M pepstain A (Sigma)). The cells are then homogenised by 2×15 second bursts in a 1 litre glass Waring blender, followed by centrifugation at 500 g for 20 minutes. The supernatant is then spun at 48,000 g for 30 minutes. The pellet is resuspended in homogenisation buffer (4× the volume of the original cell pellet) by vortexing for 5 seconds, followed by homogenisation in a Dounce homogeniser (10-15 strokes). At this point the preparation is aliquoted into polypropylene tubes and stored at −80° C.

A histamine H1 cell line may be generated in accordance with the following procedure:

(iii) Generation of Histamine H1 Cell Line

The human H1 receptor was cloned using known procedures described in the literature [Biochem. Biophys. Res. Commun. 1994, 201(2), 894]. Chinese hamster ovary cells stably expressing the human H1 receptor were generated according to known procedures described in the literature [Br. J. Pharmacol. 1996, 117(6), 1071].

Compounds of the invention may be tested for in vitro biological activity in accordance with the following assays:

(I) Histamine H3 Functional Antagonist Assay (Method A)

For each compound being assayed, in a solid white 384 well plate, is added:—

(a) 5 μl of test compound diluted to the required concentration in 10% DMSO (or 5 μl 10% DMSO as a control); and

(b) 30 μl bead/membrane/GDP mix prepared by mixing Wheat Germ Agglutinin Polystyrene LeadSeeker® (WGA PS LS) scintillation proximity assay (SPA) beads with membrane (prepared in accordance with the methodology described above) and diluting in assay buffer (20 mM N-2-Hydroxyethylpiperazine-N′-2-ethanesulfonic acid (HEPES)+100 mM NaCl+10 mM MgCl₂, pH7.4 NaOH) to give a final volume of 30 μl which contains 5 μg protein and 0.25 mg bead per well, incubating at 4° C. for 30 minutes on a roller and, just prior to addition to the plate, adding 10 μM final concentration of guanosine 5′ diphosphate (GDP) (Sigma; diluted in assay buffer).

The plates were then incubated at room temperature for 30 minutes on a shaker followed by addition of:

(c) 15 μl 0.38 nM [³⁵S]-GTPγS (Amersham; Radioactivity concentration=37 MBq/ml;

Specific activity=1160Ci/mmol), histamine (at a concentration that results in the final assay concentration of histamine being EC₈₀).

After 2-6 hours, the plate is centrifuged for 5 min at 1500 rpm and counted on a Viewlux counter using a 613/55 filter for 5 min/plate. Data is analysed using a 4-parameter logistical equation. Basal activity used as minimum i.e. histamine not added to well.

(II) Histamine H3 Functional Antagonist Assay (Method B)

For each compound being assayed, in a solid white 384 well plate, is added:—

(a) 0.5 μl of test compound diluted to the required concentration in DMSO (or 0.5 μl DMSO as a control);

(b) 30 μl bead/membrane/GDP mix prepared by mixing Wheat Germ Agglutinin Polystyrene LeadSeeker® (WGA PS LS) scintillation proximity assay (SPA) beads with membrane (prepared in accordance with the methodology described above) and diluting in assay buffer (20 mM N-2-Hydroxyethylpiperazine-N′-2-ethanesulfonic acid (HEPES)+100 mM NaCl+10 mM MgCl₂, pH7.4 NaOH) to give a final volume of 30 μl which contains 5 μg protein and 0.25 mg bead per well, incubating at room temperature for 60 minutes on a roller and, just prior to addition to the plate, adding 10 μM final concentration of guanosine 5′ diphosphate (GDP) (Sigma; diluted in assay buffer);

(c) 15 μl 0.38 nM [³⁵S]-GTPγS (Amersham; Radioactivity concentration=37 MBq/ml; Specific activity=1160 Ci/mmol), histamine (at a concentration that results in the final assay concentration of histamine being EC₈₀).

After 2-6 hours, the plate is centrifuged for 5 min at 1500 rpm and counted on a Viewlux counter using a 613/55 filter for 5 min/plate. Data is analysed using a 4-parameter logistical equation. Basal activity used as minimum i.e. histamine not added to well.

(III) Histamine H1 Functional Antagonist Assay

The histamine H1 cell line was seeded into non-coated black-walled clear bottom 384-well tissue culture plates in alpha minimum essential medium (Gibco /Invitrogen, cat no. 22561-021), supplemented with 10% dialysed foetal calf serum (Gibco/Invitrogen cat no. 12480-021) and 2 mM L-glutamine (Gibco/Invitrogen cat no 25030-024) and maintained overnight at 5% CO₂, 37° C.

Excess medium was removed from each well to leave 10 μl. 30 μl loading dye (250 μM Brilliant Black, 2 μM Fluo-4 diluted in Tyrodes buffer+probenecid (145 mM NaCl, 2.5 mM KCl, 10 mM HEPES, 10 mM D-glucose, 1.2 mM MgCl₂, 1.5 mM CaCl₂, 2.5 mM probenecid, pH adjusted to 7.40 with NaOH 1.0 M)) was added to each well and the plates were incubated for 60 minutes at 5% CO₂, 37° C.

10 μl of test compound, diluted to the required concentration in Tyrodes buffer+probenecid (or 10 μl Tyrodes buffer+probenecid as a control) was added to each well and the plate incubated for 30 min at 37° C., 5% CO₂. The plates were then placed into a FLIPR™ (Molecular Devices, UK) to monitor cell fluorescence (λₑₓ=488 nm, λ_{EM}=540 nm) in the manner described in Sullivan et al., (In: Lambert DG (ed.), Calcium Signaling Protocols, New Jersey: Humana Press, 1999, 125-136) before and after the addition of 10 μl histamine at a concentration that results in the final assay concentration of histamine being EC₈₀.

Functional antagonism is indicated by a suppression of histamine induced increase in fluorescence, as measured by the FLIPR™ system (Molecular Devices). By means of concentration effect curves, functional affinities are determined using standard pharmacological mathematical analysis.

Results

The compounds of Examples E1, E3-9, E11-47, E49-51, E53-56, E58-193, E195-219, E222-231 and E233-245 were tested in the histamine H3 functional antagonist assay (method A). These compounds exhibited antagonism>6.5 pK_{b}. More particularly, the compounds of Examples E1, E3, E4, E6-8, E11-18, E29, E43, E81, E87, E91, E96, E98, E100, E105, E108, E111, E115, E120-121, E128, E136, E140-142, E144, E147, E160-161, E165-166, E169, E171, E178, E180, E184, E192, E205-219, E222-229, E233-234, E236-238, E240 and E242-245 exhibited antagonism>9.0 pK_{b}.

The compounds of Examples E207, E220 and E221 were tested in the histamine H3 functional antagonist assay (method B). These compounds exhibited antagonism>8.5 PK_{b}.

The compounds of Examples E1, E3-9, E11-47, E49-51, E53-97, E102, E105, E107, E110, E115, E120, E129, E131, E134, E142, E155, E191-192, E195, E197-199, E201, E204-231 and E233-245 were tested in the histamine H1 functional antagonist assay and exhibited antagonism<6.3 pK_{b}.

What is claimed is:

1-9. (canceled)

10. A compound of formula (I), a pharmaceutically acceptable salt, or solvate thereof: 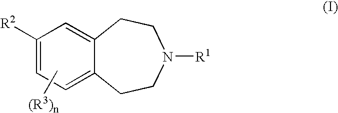
wherein:
R¹ represents —C₃₋₇ cycloalkyl optionally substituted by C₁₋₃ alkyl;
R² represents -aryl, -heterocyclyl, -heteroaryl, -aryl-X—C₃₋₈ cycloalkyl, -aryl-X-aryl, -aryl-X-heteroaryl, -aryl-X-heterocyclyl, -heteroaryl-X—C₃₋₈ cycloalkyl, -heteroaryl-X-aryl, -heteroaryl-X-heteroaryl, -heteroaryl-X-heterocyclyl, -heterocyclyl-X—C₃₋₈ cycloalkyl, -heterocyclyl-X-aryl, -heterocyclyl-X-heteroaryl, or -heterocyclyl-X-heterocyclyl;
X represents a bond, O, CO, —CH₂O—, —COCH₂—, —COCH₂O—, —CONR^{2b}—, —COCH₂NR^{2b} CO—, —CSNH—, SO₂, —SO₂C₁₋₃ alkyl-, —SO₂C₂₋₃ alkenyl-, —COC₂₋₃ alkenyl-, —CO—C(R^{2a})(R^{2b})—, or —CO—C(R^{2a})(R^{2b})CH₂—;
R^{2a} represents hydrogen or C₁₋₆ alkyl;
R^{2b} represents hydrogen, C₁₋₆ alkyl, aryl, heteroaryl, heterocyclyl, or C₁₋₆ alkylamido;
R³ represents halogen, C₁₋₆ alkyl, C₁₋₆ alkoxy, cyano, amino, or trifluoromethyl;
n is 0, 1, or 2;
wherein said alkyl, cycloalkyl, aryl, heteroaryl and heterocyclyl groups of R² may be optionally substituted by one or more substituents which may be the same or different, and which are selected from the group consisting of halogen, hydroxy, cyano, nitro, ═O, haloC₁₋₆ alkyl, haloC₁₋₆ alkoxy, C₁₋₆ alkyl, hydroxyC₁₋₆ alkyl, C₁₋₆ alkoxy, arylC₁₋₆ alkoxy, C₁₋₆ alkylthio, C₁₋₆ alkoxyC₁₋₆ alkyl, C₃₋₇ cycloalkylC₁₋₆ alkoxy, C₁₋₆ alkanoyl, C₁₋₆ alkoxycarbonyl, C₁₋₆ alkylsulfonyl, C₁₋₆ alkylsulfinyl, C₁₋₆ alkylsulfonyloxy, C₁₋₆ alkylsulfonylC₁₋₆ alkyl, sulfonyl, arylsulfonyl, arylsulfonyloxy, arylsulfonylC₁₋₆ alkyl, aryloxy, C₁₋₆ alkylsulfonamido, C₁₋₆ alkylamino, C₁₋₆ alkylamido, —R⁵, —CO₂R⁵, —COR⁵, —C₁₋₆ alkyl-COR⁵, C₁₋₆ alkylsulfonamidoC₁₋₆ alkyl, C₁₋₆ alkylamidoC₁₋₆ alkyl, arylsulfonamido, arylcarboxamido, arylsulfonamidoC₁₋₆ alkyl, arylcarboxamidoC₁₋₆ alkyl, aroyl, arylC₁₋₆ alkyl, aroylC₁₋₆ alkyl, arylC₁₋₆ alkanoyl, or a group —NR⁶R⁷, —C₁₋₆ alkyl-NR⁶R⁷, —C₃₋₈ cycloalkyl-NR⁶R⁷, —CONR⁶R⁷, NR⁶COR⁷, —NR⁶SO₂R⁷, —OCONR⁶R⁷, —NR⁶CO₂R⁷, —NR⁵CONR⁶R⁷, and —SO₂NR⁶R⁷,
wherein said R⁵, R⁶, and R⁷ independently represent hydrogen, C₁₋₆ alkyl, haloC₁₋₆ alkyl, —C₃₋₈ cycloalkyl, —C₁₋₆ alkyl-C₃₋₈ cycloalkyl, aryl, —C₁₋₆ alkyl-aryl, heterocyclyl, or heteroaryl; or wherein —NR⁶R⁷ may represent a nitrogen containing heterocyclyl group, and wherein said R⁵, R⁶ and R⁷ groups may be optionally substituted by one or more substituents which may be the same or different, and which are selected from the group consisting of halogen, hydroxy, C₁₋₆ alkyl, C₁₋₆ alkoxy, cyano, amino, ═O, and trifluoromethyl.

11. A compound according to claim 10 selected from the group consisting of:
3-[4-(3-cyclobutyl-2,3,4,5-tetrahydro-1H-3-benzazepin-7-yl)-1-piperazinyl]benzonitrile;
4-[4-(3-cyclobutyl-2,3,4,5-tetrahydro-1H-3-benzazepin-7-yl)-1-piperazinyl]benzonitrile;
2-[4-(3-cyclobutyl-2,3,4,5-tetrahydro-1H-3-benzazepin-7-yl)-1-piperazinyl]benzonitrile;
7-{4-[(2-chlorophenyl)carbonyl]-1-piperazinyl}-3-cyclobutyl-2,3,4,5-tetrahydro-1H-3-benzazepine;
3-cyclobutyl-7-[4-(tetrahydro-2H-pyran-4-ylcarbonyl)-1-piperazinyl]-2,3,4,5-tetrahydro-1H-3-benzazepine;
4-{[4-(3-cyclobutyl-2,3,4,5-tetrahydro-1H-3-benzazepin-7-yl)-1-piperazinyl]carbonyl}benzonitrile;
7-[4-(2,1,3-benzoxadiazol-5-ylcarbonyl)-1-piperazinyl]-3-cyclobutyl-2,3,4,5-tetrahydro-1H-3-benzazepine;
7-{4-[(2-chlorophenyl)sulfonyl]-1-piperazinyl}-3-cyclobutyl-2,3,4,5-tetrahydro-1H-3-benzazepine;
3-cyclobutyl-7-[4-(4-morpholinylcarbonyl)-1-piperazinyl]-2,3,4,5-tetrahydro-1H-3-benzazepine;
4-(3-cyclobutyl-2,3,4,5-tetrahydro-1H-3-benzazepin-7-yl)-N-phenyl-1-piperazinecarboxamide;
4-(3-cyclobutyl-2,3,4,5-tetrahydro-1H-3-benzazepin-7-yl)-N-[4-(methyloxy)phenyl]-1-piperazinecarboxamide;
4-(3-cyclobutyl-2,3,4,5-tetrahydro-1H-3-benzazepin-7-yl)-N-[3-(methyloxy)phenyl]-1-piperazinecarboxamide;
N-(4-chlorophenyl)-4-(3-cyclobutyl-2,3,4,5-tetrahydro-1H-3-benzazepin-7-yl)-1-piperazinecarboxamide;
4-(3-cyclobutyl-2,3,4,5-tetrahydro-1H-3-benzazepin-7-yl)-N-(4-ethylphenyl)-1-piperazinecarboxamide;
1-(4-{[4-(3-cyclobutyl-2,3,4,5-tetrahydro-1H-3-benzazepin-7-yl)-1-piperazinyl]carbonyl}phenyl)-2,2,2-trifluoroethanone;
1-(4-{[4-(3-cyclobutyl-2,3,4,5-tetrahydro-1H-3-benzazepin-7-yl)-1-piperazinyl]carbonyl}phenyl)-1-propanone;
3-cyclobutyl-7-(4-{[4-(3-pyridinyl)phenyl]carbonyl}-1-piperazinyl)-2,3,4,5-tetrahydro-1H-3-benzazepine;
3-cyclobutyl-7-{4-[(2-methyl-5-phenyl-1,3-thiazol-4-yl)carbonyl]-1-piperazinyl}-2,3,4,5-tetrahydro-1H-3-benzazepine;
3-cyclopentyl-7-{4-[(3,4-dichlorophenyl)sulfonyl]-1-piperazinyl}-2,3,4,5-tetrahydro-1H-3-benzazepine;
3-cyclopentyl-7-[4-(2-thienylsulfonyl)-1-piperazinyl]-2,3,4,5-tetrahydro-1H-3-benzazepine;
4-{[4-(3-cyclopentyl-2,3,4,5-tetrahydro-1H-3-benzazepin-7-yl)-1-piperazinyl]sulfonyl}benzonitrile;
3-cyclopentyl-7-{4-[(4-methyl-3,4-dihydro-2H-1,4-benzoxazin-7-yl)sulfonyl]-1-piperazinyl}-2,3,4,5-tetrahydro-1H-3-benzazepine;
3-cyclopentyl-7-(4-{[4-(phenyloxy)phenyl]sulfonyl}-1-piperazinyl)-2,3,4,5-tetrahydro-1H-3-benzazepine;
3-cyclopentyl-7-[4-(2,3-dihydro-1,4-benzodioxin-6-ylsulfonyl)-1-piperazinyl]-2,3,4,5-tetrahydro-1H-3-benzazepine;
7-(4-{[3,4-bis(methyloxy)phenyl]sulfonyl}-1-piperazinyl)-3-cyclopentyl-2,3,4,5-tetrahydro-1H-3-benzazepine;
3-cyclopentyl-7-(4-{[3-(methyloxy)phenyl]sulfonyl}-1-piperazinyl)-2,3,4,5-tetrahydro-1H-3-benzazepine;
3-cyclopentyl-7-(4-{[4-(methyloxy)phenyl]sulfonyl}-1-piperazinyl)-2,3,4,5-tetrahydro-1H-3-benzazepine;
2,6-dichloro-4-{[4-(3-cyclopentyl-2,3,4,5-tetrahydro-1H-3-benzazepin-7-yl)-1-piperazinyl]sulfonyl}phenol;
3-cyclopentyl-7-[4-(8-quinolinylsulfonyl)-1-piperazinyl]-2,3,4,5-tetrahydro-1H-3-benzazepine;
3-cyclopentyl-7-[4-(5-isoquinolinylsulfonyl)-1-piperazinyl]-2,3,4,5-tetrahydro-1H-3-benzazepine;
3-cyclopentyl-7-{4-[(1-methyl-1H-imidazol-4-yl)sulfonyl]-1-piperazinyl}-2,3,4,5-tetrahydro-1H-3-benzazepine;
3-cyclopentyl-7-{4-[(2,4-dimethyl-1,3-thiazol-5-yl)sulfonyl]-1-piperazinyl}-2,3,4,5-tetrahydro-1H-3-benzazepine;
3-cyclopentyl-7-{4-[(1,3,5-trimethyl-1H-pyrazol-4-yl)sulfonyl]-1-piperazinyl}-2,3,4,5-tetrahydro-1H-3-benzazepine;
3-cyclopentyl-7-[4-(3-thienylsulfonyl)-1-piperazinyl]-2,3,4,5-tetrahydro-1H-3-benzazepine;
7-[4-(1-benzothien-3-ylsulfonyl)-1-piperazinyl]-3-cyclopentyl-2,3,4,5-tetrahydro-1H-3-benzazepine;
4-(3-cyclopentyl-2,3,4,5-tetrahydro-1H-3-benzazepin-7-yl)-N,N-dimethyl-1-piperazinesulfonamide;
3-cyclopentyl-7-[4-(thieno[2,3-b]pyridin-2-ylsulfonyl)-1-piperazinyl]-2,3,4,5-tetrahydro-1H-3-benzazepine;3-cyclopentyl-7-{4-[(2,2,2-trifluoroethyl)sulfonyl]-1-piperazinyl}-2,3,4,5-tetrahydro-1H-3-benzazepine;
3-cyclopentyl-7-{4-[(phenylmethyl)sulfonyl]-1-piperazinyl}-2,3,4,5-tetrahydro-1H-3-benzazepine;
3-cyclopentyl-7-{4-[(1-methylethyl)sulfonyl]-1-piperazinyl}-2,3,4,5-tetrahydro-1H-3-benzazepine;
3-cyclopentyl-7-{4-[(4-methylphenyl)sulfonyl]-1-piperazinyl}-2,3,4,5-tetrahydro-1H-3-benzazepine;
7-{4-[(4-chlorophenyl)sulfonyl]-1-piperazinyl}-3-cyclopentyl-2,3,4,5-tetrahydro-1H-3-benzazepine;
7-{4-[(2-chlorophenyl)sulfonyl]-1-piperazinyl}-3-cyclopentyl-2,3,4,5-tetrahydro-1H-3-benzazepine;
7-{4-[(3-chlorophenyl)sulfonyl]-1-piperazinyl}-3-cyclopentyl-2,3,4,5-tetrahydro-1H-3-benzazepine;
3-cyclopentyl-7-{4-[(2,3-dichlorophenyl)sulfonyl]-1-piperazinyl}-2,3,4,5-tetrahydro-1H-3-benzazepine;
3-cyclopentyl-7-(4-{[4-(1,1-dimethylethyl)phenyl]sulfonyl}-1-piperazinyl)-2,3,4,5-tetrahydro-1H-3-benzazepine;
N-(4-{[4-(3-cyclopentyl-2,3,4,5-tetrahydro-1H-3-benzazepin-7-yl)-1-piperazinyl]sulfonyl}phenyl)acetamide;
1-(4-{[4-(3-cyclopentyl-2,3,4,5-tetrahydro-1H-3-benzazepin-7-yl)-1-piperazinyl]sulfonyl}phenyl)ethanone;
3-cyclopentyl-7-(4-{[2-(1-naphthalenyl)ethyl]sulfonyl}-1-piperazinyl)-2,3,4,5-tetrahydro-1H-3-benzazepine;
4-{[4-(3-cyclopentyl-2,3,4,5-tetrahydro-1H-3-benzazepin-7-yl)-1-piperazinyl]sulfonyl}benzoic acid;
3-cyclopentyl-7-(4-{[4-(trifluoromethyl)phenyl]sulfonyl}-1-piperazinyl)-2,3,4,5-tetrahydro-1H-3-benzazepine;
7-[4-(4-biphenylylsulfonyl)-1-piperazinyl]-3-cyclopentyl-2,3,4,5-tetrahydro-1H-3-benzazepine;
3-cyclopentyl-7-(4-{[5-(1,3-oxazol-5-yl)-2-thienyl]sulfonyl}-1-piperazinyl)-2,3,4,5-tetrahydro-1H-3-benzazepine;
3-cyclopentyl-7-[4-(2-naphthalenylsulfonyl)-1-piperazinyl]-2,3,4,5-tetrahydro-1H-3-benzazepine;
5-{[4-(3-cyclopentyl-2,3,4,5-tetrahydro-1H-3-benzazepin-7-yl)-1-piperazinyl]sulfonyl}-N,N-dimethyl-1-naphthalenamine;
3-cyclopentyl-7-(4-{[(E)-2-phenylethenyl]sulfonyl}-1-piperazinyl)-2,3,4,5-tetrahydro-1H-3-benzazepine;
3-cyclopentyl-7-(4-{[4-(1-methylethyl)phenyl]sulfonyl}-1-piperazinyl)-2,3,4,5-tetrahydro-1H-3-benzazepine;
7-{4-[(3-chloro-2-methylphenyl)sulfonyl]-1-piperazinyl}-3-cyclopentyl-2,3,4,5-tetrahydro-1H-3-benzazepine;
3-cyclopentyl-7-[4-(1-naphthalenylsulfonyl)-1-piperazinyl]-2,3,4,5-tetrahydro-1H-3-benzazepine;
7-{4-[(5-chloro-2-thienyl)sulfonyl]-1-piperazinyl}-3-cyclopentyl-2,3,4,5-tetrahydro-1H-3-benzazepine;
3-cyclopentyl-7-[4-(methylsulfonyl)-1-piperazinyl]-2,3,4,5-tetrahydro-1H-3-benzazepine;
3-cyclopentyl-7-(4-{[3-(trifluoromethyl)phenyl]sulfonyl}-1-piperazinyl)-2,3,4,5-tetrahydro-1H-3-benzazepine;
3-cyclopentyl-7-(4-{[5-(2-pyridinyl)-2-thienyl]sulfonyl}-1-piperazinyl)-2,3,4,5-tetrahydro-1H-3-benzazepine;
7-{4-[(4-chloro-1-benzothien-2-yl)sulfonyl]-1-piperazinyl}-3-cyclopentyl-2,3,4,5-tetrahydro-1H-3-benzazepine;
7-[4-(2,1,3-benzoxadiazol-4-ylsulfonyl)-1-piperazinyl]-3-cyclopentyl-2,3,4,5-tetrahydro-1H-3-benzazepine;
3-cyclopentyl-7-{4-[(1,2-dimethyl-1H-imidazol-4-yl)sulfonyl]-1-piperazinyl}-2,3,4,5-tetrahydro-1H-3-benzazepine;
N-(5-{[4-(3-cyclopentyl-2,3,4,5-tetrahydro-1H-3-benzazepin-7-yl)-1-piperazinyl]sulfonyl}-4-methyl-1,3-thiazol-2-yl)acetamide;
3-cyclopentyl-7-{4-[(3,5-dichlorophenyl)sulfonyl]-1-piperazinyl}-2,3,4,5-tetrahydro-1H-3-benzazepine;
3-cyclopentyl-7-[4-({4-[(trifluoromethyl)oxy]phenyl}sulfonyl)-1-piperazinyl]-2,3,4,5-tetrahydro-1H-3-benzazepine;
3-cyclopentyl-7-(4-{[2-(trifluoromethyl)phenyl]sulfonyl}-1-piperazinyl)-2,3,4,5-tetrahydro-1H-3-benzazepine;
3-cyclopentyl-7-{4-[(3,5-dimethyl-4-isoxazolyl)sulfonyl]-1-piperazinyl}-2,3,4,5-tetrahydro-1H-3-benzazepine;
3-cyclopentyl-7-(4-{[6-(phenyloxy)-3-pyridinyl]sulfonyl}-1-piperazinyl)-2,3,4,5-tetrahydro-1H-3-benzazepine;
3-cyclopentyl-7-[4-(phenylsulfonyl)-1-piperazinyl]-2,3,4,5-tetrahydro-1H-3-benzazepine;
3-cyclopentyl-7-{4-[(5-methyl-1-phenyl-1H-pyrazol-4-yl)sulfonyl]-1-piperazinyl}-2,3,4,5-tetrahydro-1H-3-benzazepine;
7-(4-{[(4-chlorophenyl)methyl]sulfonyl}-1-piperazinyl)-3-cyclopentyl-2,3,4,5-tetrahydro-1H-3-benzazepine;
3-cyclopentyl-7-[4-({[4-(trifluoromethyl)phenyl]methyl}sulfonyl)-1-piperazinyl]-2,3,4,5-tetrahydro-1H-3-benzazepine;
3-cyclopentyl-7-[4-(2,3-dihydro-1-benzofuran-5-ylsulfonyl)-1-piperazinyl]-2,3,4,5-tetrahydro-1H-3-benzazepine;
6-{[4-(3-cyclopentyl-2,3,4,5-tetrahydro-1H-3-benzazepin-7-yl)-1-piperazinyl]sulfonyl}-2H-chromen-2-one;
3-cyclopentyl-7-(4-{[5-(3-isoxazolyl)-2-thienyl]sulfonyl}-1-piperazinyl)-2,3,4,5-tetrahydro-1H-3-benzazepine;
7-[4-(2,1,3-benzothiadiazol-5-ylsulfonyl)-1-piperazinyl]-3-cyclopentyl-2,3,4,5-tetrahydro-1H-3-benzazepine;
7-(4-{[5-chloro-2-(methyloxy)phenyl]sulfonyl}-1-piperazinyl)-3-cyclopentyl-2,3,4,5-tetrahydro-1H-3-benzazepine;
3-cyclopentyl-7-{4-[(5-fluoro-2-methylphenyl)sulfonyl]-1-piperazinyl}-2,3,4,5-tetrahydro-1H-3-benzazepine;
7-{4-[(4-bromo-2-ethylphenyl)sulfonyl]-1-piperazinyl}-3-cyclopentyl-2,3,4,5-tetrahydro-1H-3-benzazepine;
7-{4-[(6-chloroimidazo[2,1-b][1,3]thiazol-5-yl)sulfonyl]-1-piperazinyl}-3-cyclopentyl-2,3,4,5-tetrahydro-1H-3-benzazepine;
3-cyclopentyl-7-(4-{[3-(5-methyl-1,3,4-oxadiazol-2-yl)phenyl]sulfonyl}-1-piperazinyl)-2,3,4,5-tetrahydro-1H-3-benzazepine;
3-cyclopentyl-7-{4-[(2,5-dimethyl-3-thienyl)sulfonyl]-1-piperazinyl}-2,3,4,5-tetrahydro-1H-3-benzazepine;
3-cyclopentyl-7-(4-{[4-methyl-2-(methyloxy)phenyl]sulfonyl}-1-piperazinyl)-2,3,4,5-tetrahydro-1H-3-benzazepine;
3-cyclopentyl-7-(4-{[2-(3-methylphenyl)ethyl]sulfonyl}-1-piperazinyl)-2,3,4,5-tetrahydro-1H-3-benzazepine;
3-cyclopentyl-7-(4-{[4-(methylsulfonyl)phenyl]sulfonyl}-1-piperazinyl)-2,3,4,5-tetrahydro-1H-3-benzazepine;
3-cyclopentyl-7-{4-[(3,4,5-trimethylphenyl)sulfonyl]-1-piperazinyl}-2,3,4,5-tetrahydro-1H-3-benzazepine;
4-{[4-(3-cyclopentyl-2,3,4,5-tetrahydro-1H-3-benzazepin-7-yl)-1-piperazinyl]carbonyl}benzonitrile;
3-cyclopentyl-7-[4-(4-pyridinylcarbonyl)-1-piperazinyl]-2,3,4,5-tetrahydro-1H-3-benzazepine;
3-cyclopentyl-7-[4-(cyclopentylcarbonyl)-1-piperazinyl]-2,3,4,5-tetrahydro-1H-3-benzazepine;
3-cyclopentyl-7-[4-(1H-indol-3-ylcarbonyl)-1-piperazinyl]-2,3,4,5-tetrahydro-1H-3-benzazepine;
3-cyclopentyl-7-(4-{[2-(phenyloxy)phenyl]carbonyl}-1-piperazinyl)-2,3,4,5-tetrahydro-1H-3-benzazepine;
3-cyclopentyl-7-(4-{[2-(methyloxy)phenyl]carbonyl}-1-piperazinyl)-2,3,4,5-tetrahydro-1H-3-benzazepine;3-cyclopentyl-7-{4-[(3,4-dichlorophenyl)carbonyl]-1-piperazinyl}-2,3,4,5-tetrahydro-1H-3-benzazepine;3-cyclopentyl-7-[4-(2-phenylpropanoyl)-1-piperazinyl]-2,3,4,5-tetrahydro-1H-3-benzazepine;3-cyclopentyl-7-(4-{[4-(1H-pyrazol-1-yl)phenyl]acetyl}-1-piperazinyl)-2,3,4,5-tetrahydro-1H-3-benzazepine;3-cyclopentyl-7-{4-[(4-methyl-2-phenyl-3-furanyl)carbonyl]-1-piperazinyl}-2,3,4,5-tetrahydro-1H-3-benzazepine;
3-cyclopentyl-7-(4-{[4-(1,1-dimethylethyl)phenyl]carbonyl}-1-piperazinyl)-2,3,4,5-tetrahydro-1H-3-benzazepine;
(3-{[4-(3-cyclopentyl-2,3,4,5-tetrahydro-1H-3-benzazepin-7-yl)-1-piperazinyl]carbonyl}phenyl)(phenyl)methanone;
3-cyclopentyl-7-[4-(2,3-dihydro-1-benzofuran-2-ylcarbonyl)-1-piperazinyl]-2,3,4,5-tetrahydro-1H-3-benzazepine;
7-[4-(4-biphenylylcarbonyl)-1-piperazinyl]-3-cyclopentyl-2,3,4,5-tetrahydro-1H-3-benzazepine;
7-{4-[(5-chloro-1-benzothien-2-yl)carbonyl]-1-piperazinyl}-3-cyclopentyl-2,3,4,5-tetrahydro-1H-3-benzazepine;
7-[4-(1-benzothien-2-ylcarbonyl)-1-piperazinyl]-3-cyclopentyl-2,3,4,5-tetrahydro-1H-3-benzazepine;
3-cyclopentyl-7-{4-[(5-methyl-4-phenyl-2-thienyl)carbonyl]-1-piperazinyl}-2,3,4,5-tetrahydro-1H-3-benzazepine;
7-[4-(1,3-benzothiazol-6-ylcarbonyl)-1-piperazinyl]-3-cyclopentyl-2,3,4,5-tetrahydro-1H-3-benzazepine;
3-cyclopentyl-7-[4-(phenylcarbonyl)-1-piperazinyl]-2,3,4,5-tetrahydro-1H-3-benzazepine;
3-cyclopentyl-7-[4-(2-naphthalenylcarbonyl)-1-piperazinyl]-2,3,4,5-tetrahydro-1H-3-benzazepine;
1-(4-{[4-(3-cyclopentyl-2,3,4,5-tetrahydro-1H-3-benzazepin-7-yl)-1-piperazinyl]carbonyl}phenyl)ethanone;
3-cyclopentyl-7-(4-{[4-(1-methylethyl)phenyl]carbonyl}-1-piperazinyl)-2,3,4,5-tetrahydro-1H-3-benzazepine;
3-cyclopentyl-7-(4-{[4-(trifluoromethyl)phenyl]carbonyl}-1-piperazinyl)-2,3,4,5-tetrahydro-1H-3-benzazepine;
3-cyclopentyl-7-[4-({3-[(trifluoromethyl)oxy]phenyl}carbonyl)-1-piperazinyl]-2,3,4,5-tetrahydro-1H-3-benzazepine;
7-(4-{[2-bromo-5-(methyloxy)phenyl]carbonyl}-1-piperazinyl)-3-cyclopentyl-2,3,4,5-tetrahydro-1H-3-benzazepine;
N-{2-[4-(3-cyclopentyl-2,3,4,5-tetrahydro-1H-3-benzazepin-7-yl)-1-piperazinyl]-2-oxoethyl}-2-methylbenzamide;
3-cyclopentyl-7-[4-(1,3-dihydro-2H-isoindol-2-ylacetyl)-1-piperazinyl]-2,3,4,5-tetrahydro-1H-3-benzazepine;
7-{4-[(3-chlorophenyl)carbonyl]-1-piperazinyl}-3-cyclopentyl-2,3,4,5-tetrahydro-1H-3-benzazepine;
7-{4-[(4-chlorophenyl)carbonyl]-1-piperazinyl}-3-cyclopentyl-2,3,4,5-tetrahydro-1H-3-benzazepine;
7-{4-[(2-chlorophenyl)carbonyl]-1-piperazinyl}-3-cyclopentyl-2,3,4,5-tetrahydro-1H-3-benzazepine;
3-cyclopentyl-7-{4-[(4-nitrophenyl)carbonyl]-1-piperazinyl}-2,3,4,5-tetrahydro-1H-3-benzazepine;
N-(4-{[4-(3-cyclopentyl-2,3,4,5-tetrahydro-1H-3-benzazepin-7-yl)-1-piperazinyl]carbonyl}phenyl)acetamide;
(4-{[4-(3-cyclopentyl-2,3,4,5-tetrahydro-1H-3-benzazepin-7-yl)-1-piperazinyl]carbonyl}phenyl)dimethylamine;
3-cyclopentyl-7-(4-{[3-(methyloxy)phenyl]carbonyl}-1-piperazinyl)-2,3,4,5-tetrahydro-1H-3-benzazepine;
3-cyclopentyl-7-[4-({4-[(1-methylethyl)oxy]phenyl}carbonyl)-1-piperazinyl]-2,3,4,5-tetrahydro-1H-3-benzazepine;
3-cyclopentyl-7-(4-{[4-(methyloxy)phenyl]carbonyl}-1-piperazinyl)-2,3,4,5-tetrahydro-1H-3-benzazepine;
7-(4-{[3-chloro-5-(methyloxy)phenyl]carbonyl}-1-piperazinyl)-3-cyclopentyl-2,3,4,5-tetrahydro-1H-3-benzazepine;
7-[4-(1,3-benzodioxol-5-ylacetyl)-1-piperazinyl]-3-cyclopentyl-2,3,4,5-tetrahydro-1H-3-benzazepine;
3-cyclopentyl-7-(4-{[5-(phenylmethyl)-2-furanyl]carbonyl}-1-piperazinyl)-2,3,4,5-tetrahydro-1H-3-benzazepine;
3-cyclopentyl-7-[4-(3-furanylcarbonyl)-1-piperazinyl]-2,3,4,5-tetrahydro-1H-3-benzazepine;
3-cyclopentyl-7-(4-{[3-(2-furanyl)phenyl]carbonyl}-1-piperazinyl)-2,3,4,5-tetrahydro-1H-3-benzazepine;
3-cyclopentyl-7-[4-(cyclopropylcarbonyl)-1-piperazinyl]-2,3,4,5-tetrahydro-1H-3-benzazepine;
3-cyclopentyl-7-[4-(3,3-dimethylbutanoyl)-1-piperazinyl]-2,3,4,5-tetrahydro-1H-3-benzazepine;
3-cyclopentyl-7-{4-[(2E)-3-phenyl-2-propenoyl]-1-piperazinyl}-2,3,4,5-tetrahydro-1H-3-benzazepine;
3-cyclopentyl-7-[4-({cis-4-[(1,1-dimethylethyl)oxy]cyclohexyl}carbonyl)-1-piperazinyl]-2,3,4,5-tetrahydro-1H-3-benzazepine;
3-cyclopentyl-7-(4-{[1-(1-methylethyl)-4-piperidinyl]carbonyl}-1-piperazinyl)-2,3,4,5-tetrahydro-1H-3-benzazepine;
1-{2-[4-(3-cyclopentyl-2,3,4,5-tetrahydro-1H-3-benzazepin-7-yl)-1-piperazinyl]-2-oxoethyl}-2-piperidinone;
3-cyclopentyl-7-(4-{[(1-methylethyl)oxy]acetyl}-1-piperazinyl)-2,3,4,5-tetrahydro-1H-3-benzazepine;
N-{(1R)-2-[4-(3-cyclopentyl-2,3,4,5-tetrahydro-1H-3-benzazepin-7-yl)-1-piperazinyl]-2-oxo-1-phenylethyl}acetamide;
3-cyclopentyl-7-{4-[1-(phenylmethyl)-L-prolyl]-1-piperazinyl}-2,3,4,5-tetrahydro-1H-3-benzazepine;
3-cyclopentyl-7-[4-(2,3-dihydro-1H-inden-2-ylcarbonyl)-1-piperazinyl]-2,3,4,5-tetrahydro-1H-3-benzazepine;
3-cyclopentyl-7-{4-[3-methyl-2-(phenylmethyl)butanoyl]-1-piperazinyl}-2,3,4,5-tetrahydro-1H-3-benzazepine;
3-cyclopentyl-7-{4-[(1,1-dioxido-3,4-dihydro-2H-1-benzothiopyran-6-yl)carbonyl]-1-piperazinyl}-2,3,4,5-tetrahydro-1H-3-benzazepine;
3-cyclopentyl-7-(4-{[4-(methylsulfonyl)phenyl]carbonyl}-1-piperazinyl)-2,3,4,5-tetrahydro-1H-3-benzazepine;
3-cyclopentyl-7-[4-(3,4-dihydro-2H-chromen-2-ylcarbonyl)-1-piperazinyl]-2,3,4,5-tetrahydro-1H-3-benzazepine;
3-cyclopentyl-7-[4-(2,3-dihydro-1-benzofuran-7-ylcarbonyl)-1-piperazinyl]-2,3,4,5-tetrahydro-1H-3-benzazepine;
3-cyclopentyl-7-(4-{[4-(3-pyridinyl)phenyl]carbonyl}-1-piperazinyl)-2,3,4,5-tetrahydro-1H-3-benzazepine;
3-cyclopentyl-7-[4-(3-quinolinylcarbonyl)-1-piperazinyl]-2,3,4,5-tetrahydro-1H-3-benzazepine;
3-cyclopentyl-7-[4-(pyrazolo[1,5-a]pyridin-3-ylcarbonyl)-1-piperazinyl]-2,3,4,5-tetrahydro-1H-3-benzazepine;
3-cyclopentyl-7-[4-(5-isoquinolinylcarbonyl)-1-piperazinyl]-2,3,4,5-tetrahydro-1H-3-benzazepine;
7-[4-(1-benzothien-3-ylcarbonyl)-1-piperazinyl]-3-cyclopentyl-2,3,4,5-tetrahydro-1H-3-benzazepine;
3-cyclopentyl-7-(4-{[5-(2-pyridinyl)-2-thienyl]carbonyl}-1-piperazinyl)-2,3,4,5-tetrahydro-1H-3-benzazepine;
3-cyclopentyl-7-{4-[(1,3-dimethyl-1H-thieno[2,3-c]pyrazol-5-yl)carbonyl]-1-piperazinyl}-2,3,4,5-tetrahydro-1H-3-benzazepine;
3-cyclopentyl-7-{4-[(4-methyl-2-phenyl-1,3-thiazol-5-yl)acetyl]-1-piperazinyl}-2,3,4,5-tetrahydro-1H-3-benzazepine;
7-[4-(1,3-benzothiazol-2-ylcarbonyl)-1-piperazinyl]-3-cyclopentyl-2,3,4,5-tetrahydro-1H-3-benzazepine;
3-cyclopentyl-7-[4-(imidazo[2,1-b][1,3]thiazol-5-ylacetyl)-1-piperazinyl]-2,3,4,5-tetrahydro-1H-3-benzazepine;
3-cyclopentyl-7-(4-{[4-(3,5-dimethyl-1H-pyrazol-1-yl)phenyl]carbonyl}-1-piperazinyl)-2,3,4,5-tetrahydro-1H-3-benzazepine;
3-cyclopentyl-7-{4-[(2,4-dimethyl-1,3-oxazol-5-yl)carbonyl]-1-piperazinyl}-2,3,4,5-tetrahydro-1H-3-benzazepine;
3-cyclopentyl-7-[4-(4-pyridinyl acetyl)-1-piperazinyl]-2,3,4,5-tetrahydro-1H -3-benzazepine;
6-{2-[4-(3-cyclopentyl-2,3,4,5-tetrahydro-1H-3-benzazepin-7-yl)-1-piperazinyl]-2-oxoethyl}-2(1H)-quinolinone;
3-cyclopentyl-7-[4-(phenylacetyl)-1-piperazinyl]-2,3,4,5-tetrahydro-1H-3-benzazepine;
3-cyclopentyl-7-(4-{[4-(1-methylethyl)phenyl]acetyl}-1-piperazinyl)-2,3,4,5-tetrahydro-1H-3-benzazepine;
3-cyclopentyl-7-[4-(2-naphthalenylacetyl)-1-piperazinyl]-2,3,4,5-tetrahydro-1H-3-benzazepine;
3-cyclopentyl-7-[4-(diphenylacetyl)-1-piperazinyl]-2,3,4,5-tetrahydro-1H-3-benzazepine;
3-cyclopentyl-7-(4-{[4-(trifluoromethyl)phenyl]acetyl}-1-piperazinyl)-2,3,4,5-tetrahydro-1H-3-benzazepine;
7-{4-[(4-chlorophenyl)acetyl]-1-piperazinyl}-3-cyclopentyl-2,3,4,5-tetrahydro-1H-3-benzazepine;
3-cyclopentyl-7-(4-{[4-(methyloxy)phenyl]acetyl}-1-piperazinyl)-2,3,4,5-tetrahydro-1H-3-benzazepine;
3-cyclopentyl-7-[4-(3-thienylacetyl)-1-piperazinyl]-2,3,4,5-tetrahydro-1H-3-benzazepine;
7-[4-(1-benzothien-4-ylacetyl)-1-piperazinyl]-3-cyclopentyl-2,3,4,5-tetrahydro-1H-3-benzazepine;
3-cyclopentyl-7-(4-{[4-(1-piperidinylcarbonyl)phenyl]carbonyl}-1-piperazinyl)-2,3,4,5-tetrahydro-1H-3-benzazepine;
7-[4-(1-benzofuran-4-ylcarbonyl)-1-piperazinyl]-3-cyclopentyl-2,3,4,5-tetrahydro-1H-3-benzazepine;
3-cyclopentyl-7-[4-(2-furanylcarbonyl)-1-piperazinyl]-2,3,4,5-tetrahydro-1H-3-benzazepine;
3-cyclopentyl-7-{4-[(2,5-dimethyl-3-furanyl)carbonyl]-1-piperazinyl}-2,3,4,5-tetrahydro-1H-3-benzazepine;
3-cyclopentyl-7-[4-(1-methyl-L-prolyl)-1-piperazinyl]-2,3,4,5-tetrahydro-1H-3-benzazepine;
3-cyclopentyl-7-{4-[(phenyloxy)acetyl]-1-piperazinyl}-2,3,4,5-tetrahydro-1H-3-benzazepine;
3-cyclopentyl-7-[4-(2-thienylcarbonyl)-1-piperazinyl]-2,3,4,5-tetrahydro-1H-3-benzazepine;
3-cyclopentyl-7-[4-(3-thienylcarbonyl)-1-piperazinyl]-2,3,4,5-tetrahydro-1H-3-benzazepine;
3-cyclopentyl-7-{4-[(2,4-dimethyl-1,3-thiazol-5-yl)acetyl]-1-piperazinyl}-2,3,4,5-tetrahydro-1H-3-benzazepine;
3-cyclopentyl-7-[4-(1,3-thiazol-5-ylcarbonyl)-1-piperazinyl]-2,3,4,5-tetrahydro-1H-3-benzazepine;
3-cyclopentyl-7-{4-[(5-phenyl-1,3-thiazol-4-yl)carbonyl]-1-piperazinyl}-2,3,4,5-tetrahydro-1H-3-benzazepine;
3-cyclopentyl-7-[4-(1,3-thiazol-4-ylcarbonyl)-1-piperazinyl]-2,3,4,5-tetrahydro-1H-3-benzazepine;
3-cyclopentyl-7-[4-(pyrazolo[1,5-a]pyrimidin-3-ylcarbonyl)-1-piperazinyl]-2,3,4,5-tetrahydro-1H-3-benzazepine;
3-cyclopentyl-7-{4-[(1-methyl-1H-indazol-3-yl)carbonyl]-1-piperazinyl}-2,3,4,5-tetrahydro-1H-3-benzazepine;
7-[4-(2,1,3-benzoxadiazol-5-ylcarbonyl)-1-piperazinyl]-3-cyclopentyl-2,3,4,5-tetrahydro-1H-3-benzazepine;
3-cyclopentyl-7-{4-[(1-methyl-1H-pyrazol-5-yl)carbonyl]-1-piperazinyl}-2,3,4,5-tetrahydro-1H-3-benzazepine;
3-cyclopentyl-7-{4-[(1-methyl-3-phenyl-1H-pyrazol-4-yl)carbonyl]-1-piperazinyl}-2,3,4,5-tetrahydro-1H-3-benzazepine;
3-cyclopentyl-7-{4-[(3,5-dimethyl-4-isoxazolyl)carbonyl]-1-piperazinyl}-2,3,4,5-tetrahydro-1H-3-benzazepine;
3-cyclopentyl-7-{4-[(4-methyl-1,2,5-oxadiazol-3-yl)acetyl]-1-piperazinyl}-2,3,4,5-tetrahydro-1H-3-benzazepine;
3-cyclopentyl-7-{4-[(1-methyl-1H-imidazol-2-yl)acetyl]-1-piperazinyl}-2,3,4,5-tetrahydro-1H-3-benzazepine;
3-cyclopentyl-7-{4-[(1-methyl-1H-imidazol-2-yl)carbonyl]-1-piperazinyl}-2,3,4,5-tetrahydro-1H-3-benzazepine;
4-(3-cyclopentyl-2,3,4,5-tetrahydro-1H-3-benzazepin-7-yl)-N-2-thienyl-1-piperazinecarboxamide;
4-{[4-(3-cyclobutyl-2,3,4,5-tetrahydro-1H-3-benzazepin-7-yl)-1-piperidinyl]carbonyl}benzonitrile;
4-({[(5R)-3-(3-cyclobutyl-2,3,4,5-tetrahydro-1H-3-benzazepin-7-yl)-2-oxo-1,3-oxazolidin-5-yl]methyl}oxy)benzonitrile;
4-(3-cyclobutyl-2,3,4,5-tetrahydro-1H-3-benzazepin-7-yl)benzonitrile;
4-(3-cyclobutyl-2,3,4,5-tetrahydro-1H-3-benzazepin-7-yl)-N-methylbenzamide;
1-[4-(3-cyclobutyl-2,3,4,5-tetrahydro-1H-3-benzazepin-7-yl)phenyl]-2-propanone;
2-[4-(3-cyclobutyl-2,3,4,5-tetrahydro-1H-3-benzazepin-7-yl)phenyl]-N-methylacetamide;
6-(3-cyclobutyl-2,3,4,5-tetrahydro-1H-3-benzazepin-7-yl)-N-methyl-3-pyridinecarboxamide;
3-cyclobutyl-7-[5-(4-morpholinylcarbonyl)-2-pyridinyl]-2,3,4,5-tetrahydro-1H-3-benzazepine;
3-(3-cyclobutyl-2,3,4,5-tetrahydro-1H-3-benzazepin-7-yl)-N-methylbenzamide;
5-(3-cyclobutyl-2,3,4,5-tetrahydro-1H-3-benzazepin-7-yl)-2-furancarbonitrile;
3-cyclobutyl-7-(1,3-thiazol-2-yl)-2,3,4,5-tetrahydro-1H-3-benzazepine;
4-(3-cyclobutyl-2,3,4,5-tetrahydro-1H-3-benzazepin-7-yl)-N,3-dimethylbenzamide;
4-(3-cyclobutyl-2,3,4,5-tetrahydro-1H-3-benzazepin-7-yl)-3-fluoro-N-methylbenzamide;
6-{[4-(3-cyclobutyl-2,3,4,5-tetrahydro-1H-3-benzazepin-7-yl)phenyl]oxy}-N-methyl-3-pyridinecarboxamide;
3-cyclobutyl-7-[1-(tetrahydro-2H-pyran-4-ylcarbonyl)-4-piperidinyl]-2,3,4,5-tetrahydro-1H-3-benzazepine;
5-{[4-(3-cyclobutyl-2,3,4,5-tetrahydro-1H-3-benzazepin-7-yl)-1-piperidinyl]carbonyl}-2-pyridinecarbonitrile;
3-{[4-(3-cyclobutyl-2,3,4,5-tetrahydro-1H-3-benzazepin-7-yl)-1-piperidinyl]carbonyl}benzonitrile;
3-cyclobutyl-7-[1-(2-pyrazinylcarbonyl)-4-piperidinyl]-2,3,4,5-tetrahydro-1H-3-benzazepine;
3-cyclobutyl-7-{1-[(4-fluorophenyl)carbonyl]-4-piperidinyl}-2,3,4,5-tetrahydro-1H-3-benzazepine;
7-[1-(2,1,3-benzoxadiazol-5-ylcarbonyl)-4-piperidinyl]-3-cyclobutyl-2,3,4,5-tetrahydro-1H-3-benzazepine;
3-cyclobutyl-7-(1-{[6-(trifluoromethyl)-3-pyridinyl]carbonyl}-4-piperidinyl)-2,3,4,5-tetrahydro-1H-3-benzazepine;
3-cyclobutyl-7-[1-(1,3-thiazol-4-ylcarbonyl)-4-piperidinyl]-2,3,4,5-tetrahydro-1H-3-benzazepine;
3-cyclobutyl-7-{1-[(2-fluorophenyl)carbonyl]-4-piperidinyl}-2,3,4,5-tetrahydro-1H-3-benzazepine;
3-cyclobutyl-7-[1-(2-pyridinylcarbonyl)-4-piperidinyl]-2,3,4,5-tetrahydro-1H-3-benzazepine;
3-cyclobutyl-7-[1-(3-pyridinylcarbonyl)-4-piperidinyl]-2,3,4,5-tetrahydro-1H-3-benzazepine;
3-cyclobutyl-7-[1-(pyrazolo[1,5-a]pyrimidin-3-ylcarbonyl)-4-piperidinyl]-2,3,4,5-tetrahydro-1H-3-benzazepine;
3-cyclobutyl-7-[1-(6-quinoxalinylcarbonyl)-4-piperidinyl]-2,3,4,5-tetrahydro-1H-3-benzazepine;
3-cyclobutyl-7-[1-(5-quinoxalinylcarbonyl)-4-piperidinyl]-2,3,4,5-tetrahydro-1H-3-benzazepine;
3-cyclobutyl-7-{1-[(6-methyl-3-pyridinyl)carbonyl]-4-piperidinyl}-2,3,4,5-tetrahydro-1H-3-benzazepine;
3-cyclobutyl-7-[1-(6-methyl-3-pyridinyl)-4-piperidinyl]-2,3,4,5-tetrahydro-1H-3-benzazepine;
3-cyclobutyl-7-{1-[6-(trifluoromethyl)-3-pyridinyl]-4-piperidinyl}-2,3,4,5-tetrahydro-1H-3-benzazepine;
3-[4-(3-cyclobutyl-2,3,4,5-tetrahydro-1H-3-benzazepin-7-yl)-1-piperidinyl]benzonitrile;
4-[4-(3-cyclobutyl-2,3,4,5-tetrahydro-1H-3-benzazepin-7-yl)-1-piperidinyl]benzonitrile;
6-[4-(3-cyclobutyl-2,3,4,5-tetrahydro-1H-3-benzazepin-7-yl)-1-piperidinyl]-N-methyl-3-pyridinecarboxamide;
5-[4-(3-cyclobutyl-2,3,4,5-tetrahydro-1H-3-benzazepin-7-yl)-1-piperidinyl]-N-methyl-2-pyrazinecarboxamide;
5-[4-(3-cyclobutyl-2,3,4,5-tetrahydro-1H-3-benzazepin-7-yl)-1-piperidinyl]-N-methyl-2-pyridinecarboxamide;
1-{6-[4-(3-cyclobutyl-2,3,4,5-tetrahydro-1H-3-benzazepin-7-yl)-1-piperidinyl]-3-pyridinyl}-2-pyrrolidinone;
3-cyclobutyl-7-(4-pyridinyl)-2,3,4,5-tetrahydro-1H-3-benzazepine;
6-[4-(3-cyclopentyl-2,3,4,5-tetrahydro-1H-3-benzazepin-7-yl)-1-piperidinyl]-N-methyl-3-pyridinecarboxamide;
3-cyclopentyl-7-{1-[6-(trifluoromethyl)-3-pyridinyl]-4-piperidinyl}-2,3,4,5-tetrahydro-1H-3-benzazepine;
methyl 5-[4-(3-cyclopentyl-2,3,4,5-tetrahydro-1H-3-benzazepin-7-yl)-1-piperidinyl]-2-pyrazinecarboxylate;
5-[4-(3-cyclopentyl-2,3,4,5-tetrahydro-1H-3-benzazepin-7-yl)-1-piperidinyl]-N-methyl-2-pyrazinecarboxamide;
4-({4-[3-(2-methylcyclopentyl)-2,3,4,5-tetrahydro-1H-3-benzazepin-7-yl]-1-piperidinyl}carbonyl)benzonitrile;
4-{[4-(3-cyclohexyl-2,3,4,5-tetrahydro-1H-3-benzazepin-7-yl)-1-piperidinyl]carbonyl}benzonitrile;
4-{[4-(3-cyclopentyl-2,3,4,5-tetrahydro-1H-3-benzazepin-7-yl)-1-piperidinyl]carbonyl}benzonitrile;
N-(4-cyanophenyl)-4-(3-cyclobutyl-2,3,4,5-tetrahydro-1H-3-benzazepin-7-yl)-1-piperidinecarboxamide;
N-(4-cyanophenyl)-4-(3-cyclobutyl-2,3,4,5-tetrahydro-1H-3-benzazepin-7-yl)-1-piperidinecarbothioamide;
3-cyclobutyl-7-[4-(1-naphthalenylcarbonyl)-1-piperazinyl]-2,3,4,5-tetrahydro-1H-3-benzazepine;
4-(3-cyclobutyl-2,3,4,5-tetrahydro-1H-3-benzazepin-7-yl)-N-(4-fluorophenyl)-1-piperazinecarboxamide;
4-(3-cyclobutyl-2,3,4,5-tetrahydro-1H-3-benzazepin-7-yl)-N-[4-(methyloxy)phenyl]-1-piperidinecarboxamide;
N-(3-cyanophenyl)-4-(3-cyclobutyl-2,3,4,5-tetrahydro-1H-3-benzazepin-7-yl)-1-piperidinecarboxamide;
3-cyclobutyl-7-[1-(1,3-dihydro-2H-isoindol-2-ylcarbonyl)-4-piperidinyl]-2,3,4,5-tetrahydro-1H-3-benzazepine;
N-(6-cyano-3-pyridinyl)-4-(3-cyclobutyl-2,3,4,5-tetrahydro-1H-3-benzazepin-7-yl)-1-piperidinecarboxamide;
4-(3-cyclobutyl-2,3,4,5-tetrahydro-1H-3-benzazepin-7-yl)-N-3-pyridinylbenzamide;
phenylmethyl 4-(3-cyclobutyl-2,3,4,5-tetrahydro-1H-3-benzazepin-7-yl)-1-piperazinecarboxylate;
phenylmethyl 4-(3-cyclopentyl-2,3,4,5-tetrahydro-1H-3-benzazepin-7-yl)-1-piperazinecarboxylate;
3-cyclopentyl-7-(1-piperazinyl)-2,3,4,5-tetrahydro-1H-3-benzazepine;
3-cyclobutyl-7-(1-piperazinyl)-2,3,4,5-tetrahydro-1H-3-benzazepine;
phenylmethyl 4-(3-cyclobutyl-2,3,4,5-tetrahydro-1H-3-benzazepin-7-yl)-3,6-dihydro-1(2H)-pyridinecarboxylate;
3-cyclobutyl-7-(4-piperidinyl)-2,3,4,5-tetrahydro-1H-3-benzazepine;
methyl 6-(3-cyclobutyl-2,3,4,5-tetrahydro-1H-3-benzazepin-7-yl)-3-pyridinecarboxylate;
6-(3-cyclobutyl-2,3,4,5-tetrahydro-1H-3-benzazepin-7-yl)-3-pyridinecarboxylic acid;
methyl 5-[4-(3-cyclobutyl-2,3,4,5-tetrahydro-1H-3-benzazepin-7-yl)-1-piperidinyl]-2-pyrazinecarboxylate;
5-[4-(3-cyclobutyl-2,3,4,5-tetrahydro-1H-3-benzazepin-7-yl)-1-piperidinyl]-2-pyrazinecarboxylic acid;
1,1-dimethylethyl 5-[4-(3-cyclobutyl-2,3,4,5-tetrahydro-1H-3-benzazepin-7-yl)-1-piperidinyl]-2-pyridinecarboxylate;
5-[4-(3-cyclobutyl-2,3,4,5-tetrahydro-1H-3-benzazepin-7-yl)-1-piperidinyl]-2-pyridinecarboxylic acid;
3-cyclobutyl-7-[1-(5-iodo-2-pyridinyl)-4-piperidinyl]-2,3,4,5-tetrahydro-1H-3-benzazepine;
3-cyclopentyl-7-(4-pyridinyl)-2,3,4,5-tetrahydro-1H-3-benzazepine;
3-cyclopentyl-7-(4-piperidinyl)-2,3,4,5-tetrahydro-1H-3-benzazepine;
5-[4-(3-cyclopentyl-2,3,4,5-tetrahydro-1H-3-benzazepin-7-yl)-1-piperidinyl]-2-pyrazinecarboxylic acid; and
(5R)-3-(3-cyclobutyl-2,3,4,5-tetrahydro-1H-3-benzazepin-7-yl)-5-(hydroxymethyl)-1,3-oxazolidin-2-one;
or a pharmaceutically acceptable salt or solvate thereof.

12. A pharmaceutical composition which comprises the compound of formula (I) as defined in claim 10 or a pharmaceutically acceptable salt or solvate thereof and a pharmaceutically acceptable carrier or excipient.

13. A method of treatment of neurological diseases which comprises administering to a host in need thereof an effective amount of a compound of formula (I) as defined in claim 1 or a pharmaceutically acceptable salt or solvate thereof.

14. A process for the preparation of a compound of formula (I) or a pharmaceutically acceptable salt or solvate thereof, which process comprises:
(a) reacting a compound of formula (II) 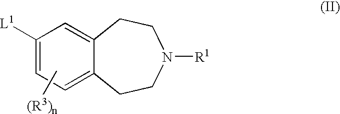
wherein R¹, R³ and n are as defined in claim 10 and L¹ represents a suitable leaving group, or an optionally activated hydroxyl group with a compound of formula R²—Y, wherein R² is as defined in claim 10 and Y represents hydrogen or a suitable coupling group; or
(b) reacting a compound of formula (III) 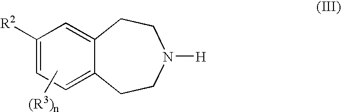
wherein R², R³ and n are as defined as in claim 10, with a compound of formula R¹-L², wherein R¹ is as defined in claim 1 and L² represents a suitable leaving group; or
(c) reacting a compound of formula (III) as defined herein, with a ketone of formula R^{1′}═O, wherein R^{1′} is C₃₋₇ cycloalkyl optionally substituted by C₁₋₃ alkyl; or
(d) preparing a compound of formula (I) wherein R² represents -heterocyclyl, wherein said heterocyclyl is a 1,3-oxazolidin-2-one group substituted at the 5-position with a hydroxymethyl group, and wherein the oxazolidin-2-one group is attached to the benzazepine moiety through the nitrogen atom, which comprises reacting a compound of formula (IV) 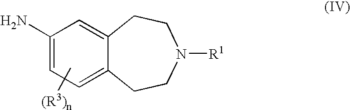
in a two step process, wherein R¹, R³ and n are as defined in claim 10, with a benzyl chloroformate group and then glycidol butyrate; or
(e) preparing a compound of formula (I) wherein R² represents -aryl, -heteroaryl, -aryl-X—C₃₋₈ cycloalkyl, -aryl-X-aryl, -aryl-X-heteroaryl, -aryl-X-heterocyclyl, -heteroaryl-X—C₃₋₈ cycloalkyl, -heteroaryl-X-aryl, -heteroaryl-X-heteroaryl, -heteroaryl-X-heterocyclyl, which comprises reacting a compound of formula (XI) 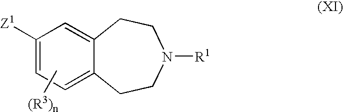
wherein R¹, R³ and n are as defined in claim 10 and Z¹ represents a suitable coupling group with a compound of formula R²″-L¹, wherein L¹ represents a suitable leaving group, or an optionally activated hydroxyl group and R^{2″} represents the groups -aryl, -heteroaryl, -aryl-X—C₃₋₈ cycloalkyl, -aryl-X-aryl, -aryl-X-heteroaryl, -aryl-X-heterocyclyl, -heteroaryl-X—C₃₋₈ cycloalkyl, -heteroaryl-X-aryl, -heteroaryl-X-heteroaryl, -heteroaryl-X-heterocyclyl, or
(f) deprotecting a compound of formula (I) which is protected; or
(g) interconversion from another compound of formula (I).
